(19) United States
(12) Patent Application Publication
(10) Pub. No.: US 2021/0147570 A1
(43) Pub. Date: May 20, 2021

(54) ANTICANCER COMBINATION THERAPY WITH CD73 ANTAGONIST ANTIBODY AND PD-1/PD-L1 AXIS ANTAGONIST ANTIBODY

(71) Applicant: BRISTOL-MYERS SQUIBB COMPANY, Princeton, NJ (US)

(72) Inventors: Rachel A. ALTURA, Belle Mead, NJ (US); Anke KLIPPEL-GIESE, Princeton, NJ (US); Jennifer R. POSTELNEK, Aberdeen, NJ (US); Yue ZHAO, Newtown, PA (US); Edward J. HILT, Yardley, PA (US); Kinjal SANGHAVI, Princeton, NJ (US); Raymond P. PEREZ, Pennington, NJ (US)

(21) Appl. No.: 17/044,753

(22) PCT Filed: Apr. 12, 2019
(86) PCT No.: PCT/US2019/027219

§ 371 Date: Oct. 1, 2020

Related U.S. Application Data
(60) Provisional application No. 62/680,255, filed on Jun. 4, 2018.
(60) Provisional application No. 62/656,892, filed on Apr. 12, 2018.

(51) Int. Cl. C07K 16/28; A61P 35/00; G01N 33/68

(52) U.S. Cl. CPC C07K 16/2896; A61P 35/00; A61K 2039/507; C07K 16/2827; G01N 33/68; C07K 16/2818

(57) ABSTRACT

Provided herein are methods for treating cancer, comprising administering to a subject having cancer a therapeutically effective amount of a CD73 antagonistic antibody alone or in combination with a PD-1/PD-L1 axis antagonist antibody.

### RELATED APPLICATIONS

This application claims the benefit of priority of U.S. Provisional Application No. 62/656,892 (filed on Apr. 12, 2018) and U.S. Provisional Application No. 62/680,255 (filed on Jun. 4, 2018). The contents of the aforementioned applications are hereby incorporated by reference in their entireties.

### BACKGROUND

Numerous antibodies which antagonize checkpoint proteins have been successfully used in cancer therapies (e.g., nivolumab). Nonetheless, some cancer patients are refractory to monotherapy with checkpoint blockade antibodies and require intervention with novel therapeutic strategies. Given the ongoing need for improved strategies for treating diseases such as cancer, novel therapies that work in conjunction with or potentiate existing therapies would be therapeutically beneficial.

### SUMMARY

Provided herein are methods of treating a subject having cancer (e.g., an advanced solid tumor) comprising administering a CD73 antagonist antibody, e.g., in combination with a PD-1/PD-L1 axis antagonist antibody.

Also provided herein is a method of treating a subject having cancer, comprising administering to the subject a therapeutically effective dose of a CD73 antagonist antibody, e.g., in combination with a PD-1/PD-L1 axis antagonist antibody, wherein the method results in one or more of the following:

(a) steady state serum concentration of the CD73 antagonist antibody is achieved 3, 4, 5, or 6 weeks after the first administration of the CD73 antagonist antibody;

(b) full receptor occupancy of the CD73 antagonist antibody, e.g., on peripheral B cells such as CD19 B cells, is achieved within 24 hours of the first administration of the CD73 antagonist antibody;

(c) full receptor occupancy of the CD73 antagonist antibody is sustained for at least 30 days after administration of the last dose of the CD73 antagonist antibody;

(d) undetectable cell surface levels of CD73 on peripheral B cells, e.g., CD19 B cells within 24 hours of the first administration of the CD73 antagonist antibody;

(e) undetectable cell surface levels of CD73 up to at least 30 days after administration of the last dose of the CD73 antagonist antibody;

(f) undetectable free soluble CD73 within 6 hours of the first administration of the CD73 antagonist antibody;

(g) undetectable free soluble CD73 at the end of the last treatment cycle (e.g., and at least 15 days or at least 30 days after the end of the last treatment cycle) including the CD73 antagonist antibody; and

(h) decrease of CD73 enzyme activity in tumor cells and/or tumor vasculature compared to before administration of the CD73 antagonist antibody.

Also provided herein is a method of treating a subject having cancer, comprising administering to the subject a combination of CD73 antagonist antibody at a fixed dose of about 150-1600 mg once every week or once every two weeks and a PD-1/PD-L1 axis antagonist antibody at a fixed dose of 240 mg or about 240 mg once every two weeks or 480 mg or about 480 mg once every four weeks, wherein the method results in one or more of the following:

(a) steady state serum concentration of the CD73 antagonist antibody is achieved 3, 4, 5, or 6 weeks after the first administration of the CD73 antagonist antibody;

(b) full receptor occupancy of the CD73 antagonist antibody, e.g., on peripheral B cells such as CD19 B cells, is achieved within 24 hours of the first administration of the CD73 antagonist antibody;

(c) full receptor occupancy of the CD73 antagonist antibody is sustained for at least 30 days after administration of the last dose of the CD73 antagonist antibody;

(d) undetectable cell surface levels of CD73 on peripheral B cells, e.g., CD19 B cells within 24 hours of the first administration of the CD73 antagonist antibody;

(e) undetectable cell surface levels of CD73 up to at least 30 days after administration of the last dose of the CD73 antagonist antibody;

(f) undetectable free soluble CD73 within 6 hours of the first administration of the CD73 antagonist antibody;

(g) undetectable free soluble CD73 at the end of the last treatment cycle (e.g., and at least 15 days or at least 30 days after the end of the last treatment cycle) including the CD73 antagonist antibody; and

(h) decrease of CD73 enzyme activity in tumor cells and/or tumor vasculature compared to before administration of the CD73 antagonist antibody.

In certain embodiments, the combination therapy with a CD73 antagonist antibody and PD-1/PD-L1 axis antagonist antibody is preceded by a monotherapy lead in phase, wherein one or more (e.g., 1-3 or 1-2) doses of the CD73 antagonist antibody are administered within 1-3 weeks (e.g., 2 weeks) prior to the first dose of the PD-1/PD-L1 axis antagonist antibody, for example, Q1W or Q2W, e.g., wherein one cycle is 2-weeks long and the monotherapy lead-in is, e.g., one cycle. In certain embodiments, a first dose of the CD73 antagonist antibody is administered 2 weeks prior to the first dose of the PD-1/PD-L1 axis antagonist antibody, and optionally, a second dose of the CD73 antagonist antibody is administered 1 week prior to the first dose of the PD-1/PD-L1 axis antagonist antibody.

In certain embodiments, the CD73 antibody and the PD-1/PD-L1 axis antagonist antibody are administered (simultaneously or sequentially) on the same day at least once.

In certain embodiments, the combination treatment with the CD73 antagonist antibody and PD-1/PD-L1 axis antagonist antibody is on a 28-day cycle, and the combination treatment consists of up to, e.g., 6 cycles or 10 cycles.

In certain embodiments, the CD73 antagonist antibody is administered at a fixed dose of about 150 mg, 300 mg, 600 mg, 1200 mg, or 1600 mg, e.g., once a week, once every two weeks, once every three weeks, or once every four weeks. In one embodiment, the CD73 antagonist antibody is administered once every week. In one embodiment, the CD73 antagonist antibody is administered once every two weeks.

In certain embodiments, the PD-1/PD-L1 axis antagonist antibody is administered once every two weeks or once every four weeks at a fixed dose of, e.g., 240 mg or about 240 mg, or 480 mg or about 480 mg. In one embodiment, the PD-1/PD-L1 axis antagonist antibody is administered once every two weeks at a fixed dose of 240 mg or about 240 mg. In one embodiment, the PD-1/PD-L1 axis antagonist antibody is administered once every four weeks at a fixed dose of 480 mg or about 480 mg.

In certain embodiments, the CD73 antagonist antibody and PD-1/PD-L1 axis antagonist antibody are formulated for intravenous or subcutaneous administration. In some embodiments, the CD73 antagonist antibody and PD-1/PD-L1 axis antagonist antibody are formulated together or separately.

In certain embodiments, steady state serum concentration of the CD73 antagonist antibody is achieved 3, 4, 5, or 6 weeks after administration of the first dose of the CD73 antagonist antibody.

In certain embodiments, target-mediated drug disposition (TMDD) saturation is achieved when the CD73 antagonist antibody is administered at a fixed dose of 600 mg or greater.

In certain embodiments, full receptor occupancy of the CD73 antagonist antibody, e.g., on peripheral B cells such as CD19 B cells, is achieved within 24 hours of administration of the first dose of the CD73 antagonist antibody when the CD73 antagonist antibody is administered at a fixed dose of 150 mg or greater. In certain embodiments, full receptor occupancy of the CD73 antagonist antibody is sustained for at least 30 days after administering the last dose of the CD73 antagonist antibody when the CD73 antagonist antibody is administered at a fixed dose of 150 mg or greater.

In certain embodiments, cell surface levels of CD73 on peripheral B cells, e.g., CD19 B cells, are undetectable within 24 hours of administration of the first dose of the CD73 antagonist antibody when the CD73 antagonist antibody is administered at a fixed dose of 150 mg or greater. In certain embodiments, cell surface levels of CD73 are undetectable for at least 30 days after administering the last dose of the CD73 antagonist when the CD73 antagonist antibody is administered at a fixed dose of 150 mg or greater.

In certain embodiments, free soluble CD73 is undetectable within 6 hours of administration of the CD73 antagonist antibody when the CD73 antagonist antibody is administered at a fixed dose of 600 mg or greater. In certain embodiments, free soluble CD73 is undetectable at the end of the last treatment cycle (or at least 15 days or at least 30 days after the end of the last treatment cycle) including the CD73 antagonist antibody when the CD73 antagonist antibody is administered at a fixed dose of 600 mg or greater.

In certain embodiments, CD73 enzyme activity is decreased in tumor cells and/or tumor vasculature compared to before administration of the CD73 antagonist antibody when the CD73 antagonist antibody is administered at a fixed dose of 150 mg or greater.

In certain embodiments, the subject has received 1, 2, 3, or 4 or more prior therapies, e.g., systemic therapies. In certain embodiments, the subject has received one or more prior immunotherapies (e.g., PD-1/PD-L1 axis antagonist therapy, e.g., nivolumab). In certain embodiments, the subject was refractory to the prior therapy. In certain embodiments, the subject progressed on or after prior cancer therapy (e.g., on or after a previous immunotherapy, such as a checkpoint inhibitor therapy (e.g., PD-1/PD-L1 axis antagonist therapy)). In certain embodiments, the previous immunotherapy was not a PD-1/PD-L1 axis antagonist therapy.

In certain embodiments, the method does not cause significant treatment-related adverse events, e.g., as determined in clinical trials.

In certain embodiments, the cancer is an advanced solid tumor, such as an advanced solid tumor that is not typically responsive to immunotherapy, e.g., not typically responsive to an anti-PD-1 or anti-PD-L1 antagonist (e.g., nivolumab).

In certain embodiments, the cancer is selected from the group consisting of colorectal cancer, ovarian cancer, renal cell carcinoma, head and neck cancer, breast cancer, pancreatic cancer, prostate cancer, gastroesophageal cancer, hepatocellular carcinoma, melanoma, anal canal epidermoid carcinoma, endometrial cancer, gastric cancer, cervical cancer, gastroesophageal junction carcinoma, alveolar soft part carcinoma, cholangiocarcinoma, esophageal cancer, intrahepatic cholangiocarcinoma, leiomyosarcoma, Merkel cell carcinoma, squamous cell anorectal carcinoma, squamous cell carcinoma of the tongue, squamous cell carcinoma of the head and neck, and urothelial cancer.

In certain embodiments, the cancer is microsatellite stable.

In certain embodiments, the treatment produces at least one therapeutic effect chosen from a reduction in size of a tumor, reduction in number of metastatic lesions over time, complete response, partial response, and stable disease.

In certain embodiments, the CD73 antagonist antibody comprises heavy chain variable region CDR1, CDR2, and CDR3 comprising the sequences set forth in SEQ ID NOs: 8, 9, and 10, respectively, and the light chain variable region CDR1, CDR2, and CDR3 comprising the sequences set forth in SEQ ID NOs: 11, 12, and 13, respectively. In certain embodiments, the CD73 antagonist antibody comprises heavy and light chain variable region sequences which are at least 80%, at least 85%, at least 90%, at least 95%, at least 98%, at least 99%, or 100% identical to the heavy and light chain variable region sequences set forth in SEQ ID NOs: 6 and 7, respectively. In certain embodiments, the CD73 antagonist antibody comprises heavy and light chain sequences which are at least 80%, at least 85%, at least 90%, at least 95%, at least 98%, at least 99%, or 100% identical to the heavy chain sequence set forth in SEQ ID NO: 3 or 4, and the light chain sequence set forth in SEQ ID NO: 5. In certain embodiments, the CD73 antagonist antibody is selected from the group consisting of an IgG1, an IgG2, an IgG3, an IgG4 or a variant or hybrid thereof. In certain embodiments, the Fc region of the CD73 antagonist antibody is an IgG2/IgG1 hybrid Fc region, such as an Fc region comprising the amino acid sequence set forth in SEQ ID NO: 14. In certain embodiments, the CD73 antagonist antibody is a human or humanized antibody.

In certain embodiments, the PD-1/PD-L1 axis antagonist antibody comprises heavy chain variable region CDR1, CDR2, and CDR3 comprising the sequences set forth in SEQ ID NOs: 20, 21, and 22 respectively, and light chain variable region CDR1, CDR2, and CDR3 comprising the sequences set forth in SEQ ID NOs: 23, 24, and 25, respectively. In certain embodiments, the PD-1/PD-L1 axis antagonist antibody comprises heavy and light chain variable region sequences which are at least 80%, at least 85%, at least 90%, at least 95%, at least 98%, at least 99%, or 100% identical to the heavy and light chain variable region sequences set forth in SEQ ID NOs: 18 and 19, respectively. In certain embodiments, the PD-1/PD-L1 axis antagonist antibody comprises heavy and light chain sequences at least 80%, at least 85%, at least 90%, at least 95%, at least 98%, at least 99%, or 100% identical to the heavy chain sequence set forth in SEQ ID NO: 15 or 16, and the light chain sequence set forth in SEQ ID NO: 17 (e.g., nivolumab).

Also provided herein is a method of treating cancer, e.g., pancreatic cancer, in a human patient, the method comprising administering to the patient an effective amount of each of:

(a) a CD73 antagonist antibody comprising a heavy chain comprising the amino acid sequence set forth in SEQ ID NO: 3 or 4, and a light chain comprising the amino acid sequence set forth in SEQ ID NO: 5, and

(b) a PD-1 antagonist antibody comprising a heavy chain comprising the amino acid sequence set forth in SEQ ID NO: 15 or 16, and a light chain comprising the amino acid sequence set forth in SEQ ID NO: 17 (e.g., nivolumab),

wherein one or more (e.g., 1-3 or 1-2) doses of the CD73 antagonist antibody are administered within 1-3 weeks (e.g., 2 weeks) prior to the first dose of the CD73 antagonist antibody, e.g., for one cycle, wherein one cycle is two weeks long, in a “CD73 antibody monotherapy lead-in,”

wherein, following the CD73 antibody monotherapy lead-in, the CD73 antagonist antibody is administered once a week at a fixed dose of about 150-1600 mg (e.g., 150 mg or about 150 mg, 300 mg or about 300 mg, 600 mg or about 600 mg, 1200 mg or about 1200 mg, 1600 mg or about 1600 mg) in combination with the PD-1 antagonist antibody, which is administered once every two weeks at a fixed dose of 240 mg or about 240 mg or once every four weeks at a fixed dose of 480 mg or about 480 mg, wherein the combination treatment consists, e.g., of up to six 28-day cycles. In certain embodiments, the patient has received one or more prior therapies (e.g., one or more prior immunotherapies) to treat the cancer. In certain embodiments, the patient progressed on the one or more prior therapies.

Also provided herein is a method of treating cancer, e.g., pancreatic cancer, in a human patient, the method comprising administering to the patient an effective amount of each of:

(a) a CD73 antagonist antibody comprising a heavy chain comprising the amino acid sequence set forth in SEQ ID NO: 3 or 4, and a light chain comprising the amino acid sequence set forth in SEQ ID NO: 5, and

(b) a PD-1 antagonist antibody comprising a heavy chain comprising the amino acid sequence set forth in SEQ ID NO: 15 or 16, and a light chain comprising the amino acid sequence set forth in SEQ ID NO: 17 (e.g., nivolumab),

wherein the CD73 antagonist antibody is administered once a week at a fixed dose of about 150-1600 mg (e.g., 150 mg or about 150 mg, 300 mg or about 300 mg, 600 mg or about 600 mg, 1200 mg or about 1200 mg, 1600 mg or about 1600 mg) in combination with the PD-1 antagonist antibody, which is administered once every two weeks at a fixed dose of 240 mg or about 240 mg or once every four weeks at a fixed dose of 480 mg or about 480 mg, wherein the combination treatment consists, e.g., of up to six 28-day cycles. In certain embodiments, the patient has received one or more prior therapies (e.g., one or more prior immunotherapies) to treat the cancer. In certain embodiments, the patient progressed on the one or more prior therapies.

Also provided herein is a method of treating cancer, e.g., pancreatic cancer, in a human patient, the method comprising administering to the patient an effective amount of each of:

(a) a CD73 antagonist antibody comprising a heavy chain comprising the amino acid sequence set forth in SEQ ID NO: 3 or 4, and a light chain comprising the amino acid sequence set forth in SEQ ID NO: 5, and

(b) a PD-1 antagonist antibody comprising a heavy chain comprising the amino acid sequence set forth in SEQ ID NO: 15 or 16, and a light chain comprising the amino acid sequence set forth in SEQ ID NO: 17 (e.g., nivolumab),

wherein one or more (e.g., 1-3 or 1-2) doses of the CD73 antagonist antibody are administered within 1-3 weeks (e.g., 2 weeks) prior to the first dose of the PD-1/PD-L1 axis antagonist antibody, e.g., for one cycle, wherein one cycle is two weeks long, in a “CD73 antibody monotherapy lead-in,”

wherein, following the CD73 antibody monotherapy lead-in, the CD73 antagonist antibody is administered once every two weeks at a fixed dose of 600 mg or about 600 mg in combination with the PD-1 antagonist antibody which is administered once every two weeks at 240 mg or about 240 mg or once every four weeks at a fixed dose of 480 mg or about 480 mg, wherein the combination therapy consists, e.g., of up to six 28-day cycles. In certain embodiments, the patient has received one or more prior therapies (e.g., one or more prior immunotherapies) to treat the cancer. In certain embodiments, the patient progressed on the one or more prior therapies.

Also provided herein is a method of treating cancer, e.g., pancreatic cancer, in a human patient, the method comprising administering to the patient an effective amount of each of:

(a) a CD73 antagonist antibody comprising a heavy chain comprising the amino acid sequence set forth in SEQ ID NO: 3 or 4, and a light chain comprising the amino acid sequence set forth in SEQ ID NO: 5, and

(b) a PD-1 antagonist antibody comprising a heavy chain comprising the amino acid sequence set forth in SEQ ID NO: 15 or 16, and a light chain comprising the amino acid sequence set forth in SEQ ID NO: 17 (e.g., nivolumab),

wherein the CD73 antagonist antibody is administered once every two weeks at a fixed dose of 600 mg or about 600 mg in combination with the PD-1 antagonist antibody, which is administered once every two weeks at 240 mg or about 240 mg or once every four weeks at a fixed dose of 480 mg or about 480 mg, wherein the combination treatment consists, e.g., of up to six 28-day cycles. In certain embodiments, the patient has received one or more prior therapies (e.g., one or more prior immunotherapies) to treat the cancer. In certain embodiments, the patient progressed on the one or more prior therapies.

In certain embodiments, the methods described herein comprise a step of first measuring the expression level of PD-L1 in the tumor of the subject with cancer, and if the expression level of PD-L1 is ≥1%, ≥5%, ≥10%, ≥25% or ≥50%, e.g., as measured with, e.g., the PD-L1 IHC 28-8 pharmDx assay, then the subject is treated with a therapeutically effective dose of the combination of the CD73 antagonist antibody and PD-1/PD-L1 axis antagonist antibody.

### BRIEF DESCRIPTION OF THE FIGURES

FIG. 1 shows staining of CD73 positive cells in tumor tissue from a prostate adenocarcinoma patient and a pancreatic adenocarcinoma patient enrolled in the clinical trial.

FIG. 2 is a schematic showing the dose escalation study design. MTD=maximum tolerated dose; NIVO=nivolumab; RP2D=recommended phase 2 dose.

FIG. 3 is a graph showing mean CD73.A serum concentrations over time when administered to patients at 150 mg, 300 mg, 600 mg, 1200 mg, and 1600 mg.

FIG. 4 is a graph showing percent receptor occupancy and CD73 cell surface protein levels on CD19 B cells in patients treated with 150 mg CD73.A Q1W+nivolumab at the indicated time points. “C0” corresponds to the CD73.A monotherapy lead-in phase. Data points for “Cell surface CD73” are indicated by dotted lines, and data points for “% RO” are indicated by solid lines. On the left side of the graph, the second dotted line for “Cell surface CD73” that extends down into “% RO” data points is pointing to a single “Cell surface CD73” data point. Similarly, on the right side of the graph, the second solid line for “% RO” that extends down into the “Cell surface CD73” data points is pointing to a single “% RO” data point.

FIG. 5 is a graph showing levels of free soluble CD73 (sCD73) at the indicated CD73.A doses (150 mg, 300 mg, 600 mg, 1200 mg, and 1500 mg) and time points (C0D1 pre, C0D1 6 hr, C0D2, C0D8, and C0D10). Rebound of sCD73 levels (“trough”) is observed at C0D8 at 150 mg and 300 mg doses of CD73.A.

FIG. 6A is an image of stained tumor sections showing CD73 enzyme activity at baseline and after treatment with 150 mg CD73.A Q1W in tumor cells and endothelial cells of a SCCHN patient (durable response). FIG. 6B is a graph showing CD73 enzyme activity in paired tumor biopsies of patients treated with the 2^{nd} indicated dose of CD73.A Q1W. CD73 enzyme activity was assessed on tumor biopsies taken on C0D10.

FIG. 7 shows images of tumor reduction in a patient with prostate cancer treated with CD73.A 300 mg and nivolumab 240 mg.

FIG. 8 shows images of tumor reduction in a patient with gastroesophageal junction carcinoma treated with CD73.A 600 mg and nivolumab 240 mg.

FIG. 9 is a graph of population PK modeling of a Q2W regimen for CD73.A at the indicated doses.

FIG. 10 is a graph showing the level of sCD73 (CD73.A bound and unbound) in patients treated with CD73.A at the indicated doses. “C” refers to “cycle”; “D” refers to “day” and “EOT” refers to “end of treatment.”

### DETAILED DESCRIPTION

Described herein are methods of treating cancer, e.g., advanced solid tumors, using isolated antibodies (e.g., isolated monoclonal antibodies) which specifically bind to CD73 and reduce CD73 activity (i.e., “CD73 antagonist antibodies”), e.g., in combination with a PD-1/PD-L1 axis antagonist antibody.

### Definitions

In order that the present description may be more readily understood, certain terms are first defined. Additional definitions are set forth throughout the detailed description.

The term “Cluster of Differentiation 73” or “CD73” as used herein refers to an enzyme (nucleotidase) capable of converting extracellular nucleoside 5′ monophosphates to nucleosides, namely adenosine monophosphate (AMP) to adenosine. CD73 is usually found as a dimer anchored to the cell membrane through a glycosylphosphatidylinositol (GPI) linkage, has ecto-enzyme activity and plays a role in signal transduction. The primary function of CD73 is its conversion of extracellular nucleotides (e.g., 5′-AMP) to adenosine, a highly immunosuppressive molecule. Thus, ecto-5′-nucleotidase catalyzes the dephosphorylation of purine and pyrimidine ribo- and deoxyribonulceoside monophosphates to the corresponding nucleoside. Although CD73 has broad substrate specificity, it prefers purine ribonucleosides.

CD73 is also referred to as ecto-5′nuclease (ecto-5′NT, EC 3.1.3.5). The term “CD73” includes any variants or isoforms of CD73 which are naturally expressed by cells.

Two isoforms of human CD73 have been identified, both of which share the same N-terminal and C-terminal portions. Isoform 1 (Accession No. NP_002517.1; SEQ ID NO: 1) represents the longest protein, consisting of 574 amino acids and 9 exons. Isoform 2 (Accession No. NP_001191742.1; SEQ ID NO: 2) encodes a shorter protein, consisting of 524 amino acids, lacking amino acids 404-453. Isoform 2 lacks an alternate in-frame exon resulting in a transcript with only 8 exons, but with the same N- and C-terminal sequences.

The terms “Programmed Death 1,” “Programmed Cell Death 1,” “Protein PD-1,” “PD-1,” PD1,” “PDCD1,” “hPD-1” and “hPD-I,” refers to an immunoinhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. U64863.

“Programmed Death Ligand-1 (PD-L1)” is one of two cell surface glycoprotein ligands for PD-1 (the other being PD-L2) that downregulate T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7.

“PD-1/PD-L1 axis antagonist antibody,” as used herein, refers to an antibody that inhibits the PD-1/PD-L1 signaling pathway by binding to PD-1 or PD-L1.

The term “antibody” as used herein may include whole antibodies and any antigen binding fragments (i.e., “antigen-binding portions”) or single chains thereof. An “antibody” refers, in one embodiment, to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. In certain naturally occurring IgG, IgD, and IgA antibodies, the heavy chain constant region is comprised of three domains, CH1, CH2, and CH3. In certain naturally occurring antibodies, each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The heavy chain of an antibody may or may not contain a terminal lysine (K), or a terminal glycine and lysine (GK). Thus, any of the heavy chain sequences and heavy chain constant region sequences provided herein can end in either GK or G, or lack K or GK, regardless of what the last amino acid of the sequence provides. This is because the terminal lysine and sometimes glycine and lysine are cleaved during expression of the antibody.

Antibodies typically bind specifically to their cognate antigen with high affinity, reflected by a dissociation constant (K_{D}) of 10⁻⁷ to 10⁻¹¹ M or less. Any K_{D} greater than about 10⁻⁶ M is generally considered to indicate nonspecific binding. As used herein, an antibody that “binds specifically” to an antigen refers to an antibody that binds to the antigen and substantially identical antigens with high affinity, which means having a K_{D} of 10⁻⁷ M or less, preferably 10⁻⁸ M or less, even more preferably 5×10⁻⁹ M or less, and most preferably between 10⁻⁸ M and 10⁻¹⁰ M or less, but does not bind with high affinity to unrelated antigens. An antigen is “substantially identical” to a given antigen if it exhibits a high degree of sequence identity to the given antigen, for example, if it exhibits at least 80%, at least 90%, at least 95%, at least 97%, or at least 99% or greater sequence identity to the sequence of the given antigen. By way of example, an antibody that binds specifically to human CD73 may also cross-react with CD73 from certain non-human primate species (e.g., cynomolgus monkey), but may not cross-react with CD73 from other species, or with an antigen other than CD73.

An immunoglobulin may be from any of the commonly known isotypes, including but not limited to IgA, secretory IgA, IgG, and IgM. The IgG isotype is divided in subclasses in certain species: IgG1, IgG2, IgG3, and IgG4 in humans, and IgG1, IgG2a, IgG2b, and IgG3 in mice. In certain embodiments, the CD73 antagonist antibodies described herein are of the human IgG1 or IgG2 subtype. Immunoglobulins, e.g., human IgG1, exist in several allotypes, which differ from each other in at most a few amino acids. “Antibody” may include, by way of example, both naturally occurring and non-naturally occurring antibodies; monoclonal and polyclonal antibodies; chimeric and humanized antibodies; human and nonhuman antibodies; wholly synthetic antibodies; and single chain antibodies.

The term “antigen-binding portion” of an antibody, as used herein, refers to one or more fragments of an antibody that retain the ability to specifically bind to an antigen (e.g., human CD73). It has been shown that the antigen-binding function of an antibody can be performed by fragments of a full-length antibody. Examples of binding fragments encompassed within the term “antigen-binding portion” of an antibody, e.g., a CD73 antagonist antibody described herein, include (i) a Fab fragment, a monovalent fragment consisting of the V_{L}, V_{H}, CL, and CH1 domains; (ii) a F(ab′)₂ fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR) or (vii) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv); see e.g., Bird et al. (1988) Science 242:423-426; and Huston et al. (1988) Proc. Nat. Acad. Sci. USA 85:5879-5883). Such single chain antibodies are also intended to be encompassed within the term “antigen-binding portion” of an antibody. These and other potential constructs are described at Chan & Carter (2010) Nat. Rev. Immunol. 10:301. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

The term “monoclonal antibody,” as used herein, refers to an antibody that displays a single binding specificity and affinity for a particular epitope or a composition of antibodies in which all antibodies display a single binding specificity and affinity for a particular epitope. Typically such monoclonal antibodies will be derived from a single cell or nucleic acid encoding the antibody, and will be propagated without intentionally introducing any sequence alterations. Accordingly, the term “human monoclonal antibody” refers to a monoclonal antibody that has variable and optional constant regions derived from human germline immunoglobulin sequences. In one embodiment, human monoclonal antibodies are produced by a hybridoma, for example, obtained by fusing a B cell obtained from a transgenic or transchromosomal non-human animal (e.g., a transgenic mouse having a genome comprising a human heavy chain transgene and a light chain transgene), to an immortalized cell.

The term “recombinant human antibody,” as used herein, includes all human antibodies that are prepared, expressed, created, or isolated by recombinant means, such as (a) antibodies isolated from an animal (e.g., a mouse) that is transgenic or transchromosomal for human immunoglobulin genes or a hybridoma prepared therefrom, (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies comprise variable and constant regions that utilize particular human germline immunoglobulin sequences and are encoded by the germline genes, but include subsequent rearrangements and mutations that occur, for example, during antibody maturation. As known in the art (see, e.g., Lonberg (2005) Nature Biotech. 23(9):1117-1125), the variable region contains the antigen binding domain, which is encoded by various genes that rearrange to form an antibody specific for a foreign antigen. In addition to rearrangement, the variable region can be further modified by multiple single amino acid changes (referred to as somatic mutation or hypermutation) to increase the affinity of the antibody to the foreign antigen. The constant region will change in further response to an antigen (i.e., isotype switch). Therefore, the rearranged and somatically mutated nucleic acid sequences that encode the light chain and heavy chain immunoglobulin polypeptides in response to an antigen may not be identical to the original germline sequences, but instead will be substantially identical or similar (i.e., have at least 80% identity).

A “human” antibody (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. The antibodies described herein may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences. The terms “human” antibodies and “fully human” antibodies and are used synonymously.

A “humanized” antibody refers to an antibody in which some, most or all of the amino acids outside the CDR domains of a non-human antibody are replaced with corresponding amino acids derived from human immunoglobulins. In one embodiment of a humanized form of an antibody, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody.

A “hybrid heavy chain constant region” refers to a heavy chain constant region comprising the constant domains CH1, hinge, CH2, and CH3, wherein one or more of the constant domains are from a different isotype (e.g. IgG1, IgG2, IgG3, IgG4). In certain embodiments, the hybrid constant region includes a human IgG2 CH1 domain and a human IgG2 hinge fused to a human IgG1 CH2 domain and a human IgG1 CH3 domain. In certain embodiments, such hybrid constant regions also include amino acid modifications within one or more of the domains relative to the wildtype amino acid sequence.

As used herein, “isotype” refers to the antibody class (e.g., IgG1, IgG2, IgG3, IgG4, IgM, IgA1, IgA2, IgD, and IgE antibody) that is encoded by the heavy chain constant region genes.

“Allotype” refers to naturally occurring variants within a specific isotype group, which variants differ in a few amino acids (see, e.g., Jefferis et al. (2009) mAbs 1:1). Antibodies described herein may be of any allotype.

Unless specified otherwise herein, all amino acid numbers are according to the EU index of the Kabat system (Kabat, E. A., et al. (1991) Sequences of Proteins of Immunological Interest, Fifth Edition, U.S. Department of Health and Human Services, NIH Publication No. 91-3242).

The phrases “an antibody recognizing an antigen” and “an antibody specific for an antigen” are used interchangeably herein with the term “an antibody which binds specifically to an antigen.”

An “isolated antibody,” as used herein, is intended to refer to an antibody that is substantially free of other antibodies having different antigenic specificities (e.g., an isolated antibody that specifically binds to CD73 is substantially free of antibodies that specifically bind antigens other than CD73). An isolated antibody that specifically binds to an epitope of CD73 may, however, have cross-reactivity to other CD73 proteins from different species.

As used herein, an antibody that “inhibits CD73” refers to an antibody that inhibits a biological and/or enzymatic function of CD73. These functions include, for example, the ability of an antibody to inhibit CD73 enzymatic activity, e.g., CD73-regulated production of adenosine or reduction of cAMP production.

As used herein, an antibody that “internalizes” refers to an antibody that crosses the cell membrane upon binding to a cell-surface antigen. Internalization includes antibody mediated receptor, e.g., CD73, internalization. In some embodiments, the antibody “internalizes” into cells expressing CD73 at a rate of T₁₂ equal to about 10 min or less.

An “effector function” refers to the interaction of an antibody Fc region with an Fc receptor or ligand, or a biochemical event that results therefrom. Exemplary “effector functions” include C1q binding, complement dependent cytotoxicity (CDC), Fc receptor binding, FcγR-mediated effector functions such as ADCC and antibody dependent cell-mediated phagocytosis (ADCP), and downregulation of a cell surface receptor (e.g., the B cell receptor; BCR). Such effector functions generally require the Fc region to be combined with a binding domain (e.g., an antibody variable domain).

The term “binds to the same epitope” with reference to two or more antibodies means that the antibodies bind to the same segment of amino acid residues, as determined by a given method. Techniques for determining whether antibodies bind to the “same epitope on CD73” with the antibodies described herein include, for example, epitope mapping methods, such as, x-ray analyses of crystals of antigen:antibody complexes, which provides atomic resolution of the epitope, and hydrogen/deuterium exchange mass spectrometry (HDX-MS). Other methods that monitor the binding of the antibody to antigen fragments (e.g. proteolytic fragments) or to mutated variations of the antigen where loss of binding due to a modification of an amino acid residue within the antigen sequence is often considered an indication of an epitope component (e.g. alanine scanning mutagenesis—Cunningham & Wells (1985) Science 244:1081). In addition, computational combinatorial methods for epitope mapping can also be used. These methods rely on the ability of the antibody of interest to affinity isolate specific short peptides from combinatorial phage display peptide libraries.

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, may be determined using known competition experiments. In certain embodiments, an antibody competes with, and inhibits binding of another antibody to a target by at least 10%, 20%, 30%, 40%, 50%, 60%, 70%, 80%, 90% or 100%. The level of inhibition or competition may be different depending on which antibody is the “blocking antibody” (i.e., the cold antibody that is incubated first with the target). Competition assays can be conducted as described, for example, in Ed Harlow and David Lane, Cold Spring Harb Protoc; 2006; doi:10.1101/pdb.prot4277 or in Chapter 11 of “Using Antibodies” by Ed Harlow and David Lane, Cold Spring Harbor Laboratory Press, Cold Spring Harbor, N.Y., USA 1999. Competing antibodies bind to the same epitope, an overlapping epitope or to adjacent epitopes (e.g., as evidenced by steric hindrance).

Other competitive binding assays include: solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using I-125 label (see Morel et al., Mol. Immunol. 25(1):7 (1988)); solid phase direct biotin-avidin EIA (Cheung et al., Virology 176:546 (1990)); and direct labeled RIA. (Moldenhauer et al., Scand. J. Immunol. 32:77 (1990)).

As used herein, the terms “specific binding,” “selective binding,” “selectively binds,” and “specifically binds,” refer to antibody binding to an epitope on a predetermined antigen but not to other antigens. Typically, the antibody (i) binds with an equilibrium dissociation constant (K_{D}) of approximately less than 10⁻⁷ M, such as approximately less than 10⁻⁸ M, 10⁻⁹ M or 10⁻¹⁰ M or even lower when determined by, e.g., surface plasmon resonance (SPR) technology in a BIACORE® 2000 surface plasmon resonance instrument using the predetermined antigen, e.g., recombinant human CD73, as the analyte and the antibody as the ligand, or Scatchard analysis of binding of the antibody to antigen positive cells, and (ii) binds to the predetermined antigen with an affinity that is at least two-fold greater than its affinity for binding to a non-specific antigen (e.g., BSA, casein) other than the predetermined antigen or a closely-related antigen. Accordingly, unless otherwise indicated, an antibody that “specifically binds to human CD73” refers to an antibody that binds to soluble or cell bound human CD73 with a K_{D} of 10⁻⁷ M or less, such as approximately less than 10⁻⁸ M, 10⁻⁹ M or 10⁻¹⁰ M or even lower. An antibody that “cross-reacts with cynomolgus CD73” refers to an antibody that binds to cynomolgus CD73 with a K_{D} of 10⁻⁷ M or less, such as less than 10⁻⁸ M, 10⁻⁹ M or 10⁻¹⁰ M or even lower. In certain embodiments, antibodies that do not cross-react with CD73 from a non-human species exhibit essentially undetectable binding against these proteins in standard binding assays.

A “rate of internalization” of an antibody or of a receptor, e.g., CD73, as mediated by the antibody, e.g., a CD73 antagonist antibody, may be represented, e.g., by T_{1/2} of internalization, e.g., as shown in the Examples. A rate of internalization of a CD73 antagonist antibody may be enhanced or increased by at least 10%, 30%, 50%, 75%, 2 fold, 3 fold, 5 fold or more, resulting in a reduction of the T_{1/2} by at least 10%, 30%, 50%, 75%, 2 fold, 3 fold, 5 fold or more by changing the heavy chain constant region of the antibody to a modified heavy chain constant region, e.g., one that contains an IgG2 hinge and IgG2 CH1 domain. For example, instead of having a T_{1/2} of 10 minutes, a modified heavy chain constant region may increase the rate of internalization and thereby reduce the T_{1/2} to 5 minutes (i.e., a two fold increase in rate of internalization or a two-fold decrease in T_{1/2}). “T_{1/2}” is defined as the time at which half of the maximal internalization is achieved, as measured from the time the antibody is added to the cells. The maximal level of internalization can be the level of internalization at the plateau of a graph representing the internalization plotted against antibody concentrations. A modified heavy chain constant region may increase the maximal level of internalization of an antibody by at least 10%, 30%, 50%, 75%, 2 fold, 3 fold, 5 fold or more. Another way of comparing internalization efficacies of different antibodies, such as an antibody with, and the same antibody without, a modified heavy chain constant region, is by comparing their level of internalization at a given antibody concentration (e.g., 100 nM) or at a given time (e.g., 2 minutes, 5 minutes, 10 minutes, or 30 minutes). Comparing levels of internalization can also be done by comparing the EC₅₀ levels of internalization. The level of internalization of one antibody can be defined relative to that of a given (reference) antibody, e.g., an antibody described herein, e.g., CD73.4-IgG2CS-IgG1.1f (also referred to herein as “CD73.A”), and, can be indicated as a percentage of the value obtained with the given (reference) antibody. The extent of internalization may be enhanced by at least 10%, 30%, 50%, 75%, 2 fold, 3 fold, 5 fold or more, as compared by any one of these methods.

A “polypeptide” refers to a chain comprising at least two consecutively linked amino acid residues, with no upper limit on the length of the chain. One or more amino acid residues in the protein may contain a modification such as, but not limited to, glycosylation, phosphorylation, or a disulfide bond. A “protein” may comprise one or more polypeptides.

The term “nucleic acid molecule,” as used herein, is intended to include DNA molecules and RNA molecules. A nucleic acid molecule may be single-stranded or double-stranded, and may be cDNA. In certain embodiments, a DNA molecule does not encompass naturally-occurring DNA molecules.

Also provided are “conservative sequence modifications” of the sequences set forth in SEQ ID NOs described herein, i.e., amino acid sequence modifications which do not abrogate the binding of the antibody encoded by the nucleotide sequence or containing the amino acid sequence, to the antigen. Such conservative sequence modifications include conservative nucleotide and amino acid substitutions, as well as, nucleotide and amino acid additions and deletions. For example, modifications can be introduced into SEQ ID NOs described herein by standard techniques known in the art, such as site-directed mutagenesis and PCR-mediated mutagenesis. Conservative sequence modifications include conservative amino acid substitutions, in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, a predicted nonessential amino acid residue in a CD73 antagonist antibody is preferably replaced with another amino acid residue from the same side chain family. Methods of identifying nucleotide and amino acid conservative substitutions that do not eliminate antigen binding are well-known in the art (see, e.g., Brummell et al., Biochem. 32:1180-1187 (1993); Kobayashi et al. Protein Eng. 12(10):879-884 (1999); and Burks et al. Proc. Natl. Acad. Sci. USA 94:412-417 (1997)).

In one embodiment, mutations can be introduced randomly along all or part of a CD73 antagonist antibody coding sequence, such as by saturation mutagenesis, and the resulting modified CD73 antagonist antibodies can be screened for improved binding activity.

For polypeptides, the term “substantial homology” indicates that two polypeptides, or designated sequences thereof, when optimally aligned and compared, are identical, with appropriate amino acid insertions or deletions, in at least about 80% of the amino acids, usually at least about 90% to 95%, and more preferably at least about 98% to 99.5% of the amino acids.

The percent identity between two sequences is a function of the number of identical positions shared by the sequences when the sequences are optimally aligned (i.e., % homology=# of identical positions/total # of positions×100), with optimal alignment determined taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

The protein sequences described herein can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to the nucleic acid molecules described herein. BLAST protein searches can be performed with the XBLAST program, score=50, wordlength=3 to obtain amino acid sequences homologous to the protein molecules described herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See www.ncbi.nlm.nih.gov.

An “immune response” refers to a biological response within a vertebrate against foreign agents, which response protects the organism against these agents and diseases caused by them. An immune response is mediated by the action of a cell of the immune system (for example, a T lymphocyte, B lymphocyte, natural killer (NK) cell, macrophage, eosinophil, mast cell, dendritic cell, or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune response or reaction includes, e.g., activation or inhibition of a T cell, e.g., an effector T cell or a Th cell, such as a CD4+ or CD8+ T cell, or the inhibition of a Treg cell.

An “immunomodulator” or “immunoregulator” refers to an agent, e.g., a component of a signaling pathway, which may be involved in modulating, regulating, or modifying an immune response. “Modulating,” “regulating,” or “modifying” an immune response refers to any alteration in a cell of the immune system or in the activity of such cell (e.g., an effector T cell). Such modulation includes stimulation or suppression of the immune system which may be manifested by an increase or decrease in the number of various cell types, an increase or decrease in the activity of these cells, or any other changes which can occur within the immune system. Both inhibitory and stimulatory immunomodulators have been identified, some of which may have enhanced function in a tumor microenvironment. The immunomodulator may be located on the surface of a T cell. An “immunomodulatory target” or “immunoregulatory target” is an immunomodulator that is targeted for binding by, and whose activity is altered by the binding of, a substance, agent, moiety, compound or molecule. Immunomodulatory targets include, for example, receptors on the surface of a cell (“immunomodulatory receptors”) and receptor ligands (“immunomodulatory ligands”).

An increased ability to stimulate an immune response, or the immune system, can result from an enhanced agonist activity of T cell costimulatory receptors and/or an enhanced antagonist activity of inhibitory receptors. An increased ability to stimulate an immune response or the immune system may be reflected by a fold increase of the EC50 or maximal level of activity in an assay that measures an immune response, e.g., an assay that measures changes in cytokine or chemokine release, cytolytic activity (determined directly on target cells or indirectly via detecting CD107a or granzymes) and proliferation. The ability to stimulate an immune response or the immune system activity may be enhanced by at least 10%, 30%, 50%, 75%, 2 fold, 3 fold, 5 fold or more.

“Immunotherapy” refers to the treatment of a subject afflicted with, or at risk of contracting or suffering a recurrence of, a disease by a method comprising inducing, enhancing, suppressing or otherwise modifying an immune response.

As used herein, “administering” refers to the physical introduction of a composition comprising a therapeutic agent to a subject, using any of the various methods and delivery systems known to those skilled in the art. Preferred routes of administration for antibodies described herein include intravenous, intraperitoneal, intramuscular, subcutaneous, spinal, or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural, and intrasternal injection and infusion, as well as in vivo electroporation. Alternatively, an antibody described herein can be administered via a non-parenteral route, such as a topical, epidermal or mucosal route of administration, for example, intranasally, orally, vaginally, rectally, sublingually, or topically. Administering can also be performed, for example, once, a plurality of times, and/or over one or more extended periods.

As used herein, the terms “inhibits” or “blocks” (e.g., referring to inhibition/blocking of CD73 binding or activity) are used interchangeably and encompass both partial and complete inhibition/blocking.

As used herein, “cancer” refers a broad group of diseases characterized by the uncontrolled growth of abnormal cells in the body. Unregulated cell division may result in the formation of malignant tumors or cells that invade neighboring tissues and may metastasize to distant parts of the body through the lymphatic system or bloodstream.

The terms “treat,” “treating,” and “treatment,” as used herein, refer to any type of intervention or process performed on, or administering an active agent to, the subject with the objective of reversing, alleviating, ameliorating, inhibiting, or slowing down or preventing the progression, development, severity, or recurrence of a symptom, complication, condition or biochemical indicia associated with a disease. Prophylaxis refers to administration to a subject who does not have a disease, to prevent the disease from occurring or minimize its effects if it does.

A “hematological malignancy” includes a lymphoma, leukemia, myeloma, or a lymphoid malignancy, as well as a cancer of the spleen and the lymph nodes. Exemplary lymphomas include both B cell lymphomas and T cell lymphomas. B-cell lymphomas include both Hodgkin's lymphomas and most non-Hodgkin's lymphomas. Non-limiting examples of B cell lymphomas include diffuse large B-cell lymphoma, follicular lymphoma, mucosa-associated lymphatic tissue lymphoma, small cell lymphocytic lymphoma (overlaps with chronic lymphocytic leukemia), mantle cell lymphoma (MCL), Burkitt's lymphoma, mediastinal large B cell lymphoma, Waldenstram macroglobulinemia, nodal marginal zone B cell lymphoma, splenic marginal zone lymphoma, intravascular large B-cell lymphoma, primary effusion lymphoma, lymphomatoid granulomatosis. Non-limiting examples of T cell lymphomas include extranodal T cell lymphoma, cutaneous T cell lymphomas, anaplastic large cell lymphoma, and angioimmunoblastic T cell lymphoma. Hematological malignancies also include leukemia, such as, but not limited to, secondary leukemia, chronic lymphocytic leukemia, acute myelogenous leukemia, chronic myelogenous leukemia, and acute lymphoblastic leukemia. Hematological malignancies further include myelomas, such as, but not limited to, multiple myeloma and smoldering multiple myeloma. Other hematological and/or B cell- or T-cell-associated cancers are encompassed by the term hematological malignancy.

The term “effective dose” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, or a prevention of impairment or disability due to the disease affliction. In reference to solid tumors, an effective amount comprises an amount sufficient to cause a tumor to shrink and/or to decrease the growth rate of the tumor (such as to suppress tumor growth) or to prevent or delay other unwanted cell proliferation. In certain embodiments, an effective amount is an amount sufficient to delay tumor development. In certain embodiments, an effective amount is an amount sufficient to prevent or delay tumor recurrence. An effective amount can be administered in one or more administrations. The effective amount of the drug or composition may: (i) reduce the number of cancer cells; (ii) reduce tumor size; (iii) inhibit, retard, slow to some extent and may stop cancer cell infiltration into peripheral organs; (iv) inhibit, i.e., slow to some extent and may stop, tumor metastasis; (v) inhibit tumor growth; (vi) prevent or delay occurrence and/or recurrence of tumor; and/or (vii) relieve to some extent one or more of the symptoms associated with the cancer. In one example, an “effective amount” is the amount of a CD73 antagonist antibody and the amount of a PD-1/PD-L1 axis antagonist antibody, in combination, clinically proven to affect a significant decrease in cancer or slowing of progression of cancer, such as an advanced solid tumor.

As used herein, the terms “fixed dose,” “flat dose,” and “flat-fixed dose” are used interchangeably and refer to a dose that is administered to a patient without regard for the weight or body surface area (BSA) of the patient. The fixed or flat dose is therefore not provided as a mg/kg dose, but rather as an absolute amount of the agent (e.g., the CD73 antagonist antibody and/or PD-1/PD-L1 axis antagonist antibody).

A “prophylactically effective amount” or a “prophylactically effective dosage” of a drug is an amount of the drug that, when administered alone or in combination with another therapeutic agent to a subject at risk of developing a disease or of suffering a recurrence of disease, inhibits the development or recurrence of the disease. The ability of a therapeutic or prophylactic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

By way of example, an anti-cancer agent is a drug that slows cancer progression or promotes cancer regression in a subject. In preferred embodiments, a therapeutically effective amount of the drug promotes cancer regression to the point of eliminating the cancer. “Promoting cancer regression” means that administering an effective amount of the drug, alone or in combination with an anti-neoplastic agent, results in a reduction in tumor growth or size, necrosis of the tumor, a decrease in severity of at least one disease symptom, an increase in frequency and duration of disease symptom-free periods, a prevention of impairment or disability due to the disease affliction, or otherwise amelioration of disease symptoms in the patient. Pharmacological effectiveness refers to the ability of the drug to promote cancer regression in the patient. Physiological safety refers to an acceptably low level of toxicity, or other adverse physiological effects at the cellular, organ, and/or organism level (adverse effects) resulting from administration of the drug.

By way of example for the treatment of tumors, a therapeutically effective amount or dosage of the drug preferably inhibits cell growth or tumor growth by at least about 20%, more preferably by at least about 40%, even more preferably by at least about 60%, and still more preferably by at least about 80% relative to untreated subjects. In the most preferred embodiments, a therapeutically effective amount or dosage of the drug completely inhibits cell growth or tumor growth, i.e., preferably inhibits cell growth or tumor growth by 100%. The ability of a compound to inhibit tumor growth can be evaluated using the assays described infra. Alternatively, this property of a composition can be evaluated by examining the ability of the compound to inhibit cell growth, such inhibition can be measured in vitro by assays known to the skilled practitioner. In other preferred embodiments described herein, tumor regression may be observed and may continue for a period of at least about 20 days, more preferably at least about 40 days, or even more preferably at least about 60 days.

The terms “patient” and “subject” refer to a human. For example, the methods and compositions described herein can be used to treat a subject or patient having cancer, such as an advanced solid tumor.

The use of the alternative (e.g., “or”) should be understood to mean either one, both, or any combination thereof of the alternatives.

As used herein, the indefinite articles “a” or “an” should be understood to refer to “one or more” of any recited or enumerated component.

As used herein, the term “about,” in the context of a numerical value or range, means±10% of the numerical value or range.

Any concentration range, percentage range, ratio range, or integer range described herein is to be understood to include the value of any integer within the recited range and, when appropriate, fractions thereof (such as one tenth and one hundredth of an integer), unless otherwise indicated.

Various aspects described herein are described in further detail in the following subsections.

### I. Methods of Treatment

Provided herein are methods of treating a subject with cancer (e.g., an advanced solid tumor) with a CD73 antagonist antibody, e.g., in combination with a PD-1/PD-L1 axis antagonist antibody. The treatments, e.g., combination treatments, described herein are useful for inhibiting tumor cell proliferation, for example, in patients who have undergone one or more previous immunotherapies (e.g., anti-PD-1 therapy). Treatment of patients with a CD73 antagonist antibody, e.g., in combination with a PD-1/PD-L1 axis antagonist antibody, can reduce tumor growth and metastasis in a patient. As described in the Examples, treating human cancer patients with a combination of a CD73 antagonist antibody (e.g., CD73.A) and a PD-1/PD-L1 axis antagonist antibody (nivolumab) results in surprisingly rapid and sustained target engagement of the CD73 antagonist antibody, CD73 internalization, loss of cell surface levels of CD73, loss of soluble CD73, loss of CD73 enzyme activity on tumor cells and tumor vasculature, as well as tumor regression.

Accordingly, provided herein is a method of treating a subject having cancer, comprising administering to the subject a therapeutically effective dose of a CD73 antagonist antibody, wherein the method results in one or more (e.g., 1, 2, 3, 4, 5, 6, 7, or 8) of the following:

(a) steady state serum concentration of the CD73 antagonist antibody is achieved 3, 4, 5, or 6 weeks after the first administration of the CD73 antagonist antibody;

(b) full receptor occupancy of the CD73 antagonist antibody, e.g., on peripheral B cells such as CD19 B cells, is achieved within 24 hours of the first administration of the CD73 antagonist antibody;

(c) full receptor occupancy of the CD73 antagonist antibody is sustained for at least 30 days after administration of the last dose of the CD73 antagonist antibody;

(d) undetectable cell surface levels of CD73 on peripheral B cells, e.g., CD19 B cells within 24 hours of the first administration of the CD73 antagonist antibody;

(e) undetectable cell surface levels of CD73 up to at least 30 days after administration of the last dose of the CD73 antagonist antibody;

(f) undetectable free soluble CD73 within 6 hours of the first administration of the CD73 antagonist antibody;

(g) undetectable free soluble CD73 at the end of the last treatment cycle including the CD73 antagonist antibody; and

(h) decrease of CD73 enzyme activity in tumor cells and/or tumor vasculature compared to before administration of the CD73 antagonist antibody.

Also provided herein is a method of treating a subject having cancer, comprising administering to the subject a therapeutically effective dose of a combination of a CD73 antagonist antibody and a PD-1/PD-L1 axis antagonist antibody, wherein the method results in one or more (e.g., 1, 2, 3, 4, 5, 6, 7, or 8) of the following:

(a) steady state serum concentration of the CD73 antagonist antibody is achieved 3, 4, 5, or 6 weeks after the first administration of the CD73 antagonist antibody;

(b) full receptor occupancy of the CD73 antagonist antibody, e.g., on peripheral B cells such as CD19 B cells, is achieved within 24 hours of the first administration of the CD73 antagonist antibody;

(c) full receptor occupancy of the CD73 antagonist antibody is sustained for at least 30 days after administration of the last dose of the CD73 antagonist antibody;

(d) undetectable cell surface levels of CD73 on peripheral B cells, e.g., CD19 B cells within 24 hours of the first administration of the CD73 antagonist antibody;

(e) undetectable cell surface levels of CD73 up to at least 30 days after administration of the last dose of the CD73 antagonist antibody;

(f) undetectable free soluble CD73 within 6 hours of the first administration of the CD73 antagonist antibody;

(g) undetectable free soluble CD73 at the end of the last treatment cycle including the CD73 antagonist antibody; and

(h) decrease of CD73 enzyme activity in tumor cells and/or tumor vasculature compared to before administration of the CD73 antagonist antibody.

Further provided herein is a method of treating a subject having cancer, comprising administering to the subject a combination of CD73 antagonist antibody at a fixed dose of about 100-1800 mg (e.g., 150-1600 mg, 150-1200 mg, 150-600 mg, 150-300 mg, 300-1600 mg, 300-1200 mg, 300-600 mg, 600 to 1200 mg, 600-1200 mg, 100 mg, 150 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, about 100 mg, about 150 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, or about 1600 mg) once every week or once every two weeks, and a PD-1/PD-L1 axis antagonist antibody at a fixed dose of 240 mg or about 240 mg once every two weeks, 360 mg or about 360 mg once every three weeks, or 480 mg or about 480 mg once every four weeks, wherein the method results in one or more of the following:

(a) steady state serum concentration of the CD73 antagonist antibody is achieved 3, 4, 5, or 6 weeks after the first administration of the CD73 antagonist antibody;

(b) full receptor occupancy of the CD73 antagonist antibody, e.g., on peripheral B cells such as CD19 B cells, is achieved within 24 hours of the first administration of the CD73 antagonist antibody;

(c) full receptor occupancy of the CD73 antagonist antibody is sustained for at least 30 days after administration of the last dose of the CD73 antagonist antibody;

(d) undetectable cell surface levels of CD73 on peripheral B cells, e.g., CD19 B cells within 24 hours of the first administration of the CD73 antagonist antibody;

(e) undetectable cell surface levels of CD73 up to at least 30 days after administration of the last dose of the CD73 antagonist antibody;

(f) undetectable free soluble CD73 within 6 hours of the first administration of the CD73 antagonist antibody;

(g) undetectable free soluble CD73 at the end of the last treatment cycle including the CD73 antagonist antibody; and

(h) decrease of CD73 enzyme activity in tumor cells and/or tumor vasculature compared to before administration of the CD73 antagonist antibody.

In certain embodiments, the combination treatment of CD73 antagonist antibody and PD-1/PD-L1 axis antagonist antibody is administered for 1-10 cycles or more (e.g., 1, 2, 3, 4, 5, 6, 7, 8, 9, or 10 or more cycles), wherein once cycle is, e.g., 28 days or 4 weeks long.

In certain embodiments, one or more (e.g., 1-3, 1-2, 1, 2, 3) doses of the CD73 antagonist antibody are administered within 1-3 weeks (e.g., 2 weeks) prior to the first dose of the PD-1/PD-L1 axis antagonist antibody (“CD73 antibody monotherapy lead-in”).

In certain embodiments, the CD73 antagonist antibody and PD-1/PD-L1 axis antagonist antibody are administered to a subject having cancer (e.g., an advanced solid tumor) at one of the following combination doses: about 100-1800 mg of the CD73 antagonist antibody Q1W and about 240-480 mg of the PD-1/PD-L1 axis antagonist antibody Q2W, about 100-1800 mg of the CD73 antagonist antibody Q2W and about 240-480 mg of the PD-1/PD-L1 axis antagonist antibody Q2W, about 100-1800 mg of the CD73 antagonist antibody Q1W and about 240-480 mg of the PD-1/PD-L1 axis antagonist antibody Q4W, about 100-1800 mg of the CD73 antagonist antibody Q2W and about 240-480 mg of the PD-1/PD-L1 axis antagonist antibody Q4W, about 150 mg of the CD73 antagonist antibody Q1W and about 240 mg of the PD-1/PD-L1 axis antagonist antibody Q2W; about 300 mg of the CD73 antagonist antibody Q1W and about 240 mg of the PD-1/PD-L1 axis antagonist antibody Q2W; about 600 mg of the CD73 antagonist antibody Q1W and about 240 mg of the PD-1/PD-L1 axis antagonist antibody Q2W; about 1200 mg of the CD73 antagonist antibody Q1W and about 240 mg of the PD-1/PD-L1 axis antagonist antibody Q2W; about 1600 mg of the CD73 antagonist antibody Q1W and about 240 mg of the PD-1/PD-L1 axis antagonist antibody Q2W; about 150 mg of the CD73 antagonist antibody Q1W and about 480 mg of the PD-1/PD-L1 axis antagonist antibody Q4W; about 300 mg of the CD73 antagonist antibody Q1W and about 480 mg of the PD-1/PD-L1 axis antagonist antibody Q4W; about 600 mg of the CD73 antagonist antibody Q1W and about 480 mg of the PD-1/PD-L1 axis antagonist antibody Q4W; about 1200 mg of the CD73 antagonist antibody Q1W and about 480 mg of the PD-1/PD-L1 axis antagonist antibody Q4W; about 1600 mg of the CD73 antagonist antibody Q1W and about 480 mg of the PD-1/PD-L1 axis antagonist antibody Q4W; about 150 mg of the CD73 antagonist antibody Q2W and about 240 mg of the PD-1/PD-L1 axis antagonist antibody Q2W; about 300 mg of the CD73 antagonist antibody Q2W and about 240 mg of the PD-1/PD-L1 axis antagonist antibody Q2W; about 600 mg of the CD73 antagonist antibody Q2W and about 240 mg of the PD-1/PD-L1 axis antagonist antibody Q2W; about 1200 mg of the CD73 antagonist antibody Q2W and about 240 mg of the PD-1/PD-L1 axis antagonist antibody Q2W; about 1600 mg of the CD73 antagonist antibody Q2W and about 240 mg of the PD-1/PD-L1 axis antagonist antibody Q2W; about 150 mg of the CD73 antagonist antibody Q2W and about 480 mg of the PD-1/PD-L1 axis antagonist antibody Q4W; about 300 mg of the CD73 antagonist antibody Q2W and about 480 mg of the PD-1/PD-L1 axis antagonist antibody Q4W; about 600 mg of the CD73 antagonist antibody Q2W and about 480 mg of the PD-1/PD-L1 axis antagonist antibody Q4W; about 1200 mg of the CD73 antagonist antibody Q2W and about 480 mg of the PD-1/PD-L1 axis antagonist antibody Q4W; about 1600 mg of the CD73 antagonist antibody Q2W and about 480 mg of the PD-1/PD-L1 axis antagonist antibody Q4W.

In certain embodiments, the CD73 antagonist antibody and PD-1/PD-L1 axis antagonist antibody are administered on the same day, e.g., Day 1 of each cycle. In certain embodiments, the CD73 antagonist antibody and PD-1/PD-L1 axis antagonist antibody are administered simultaneously (e.g., as a single formulation). Alternatively, the CD73 antagonist antibody and PD-1/PD-L1 axis antagonist antibody can be formulated for separate administration and are administered concurrently or sequentially (e.g., one antibody is administered within about 30 minutes prior to administration of the second antibody). For example, the PD-1/PD-L1 axis antagonist antibody can be administered first and followed by (e.g., immediately followed by) the administration of the CD73 antagonist antibody, or vice versa. In certain embodiments, the PD-1/PD-L1 axis antagonist antibody is administered prior to administration of the CD73 antagonist antibody. In one embodiment, the PD-1/PD-L1 axis antagonist antibody is administered after administration of the CD73 antagonist antibody. In one embodiment, the CD73 antagonist antibody and PD-1/PD-L1 axis antagonist antibody are administered concurrently. Such concurrent or sequential administration may result in both antibodies being simultaneously present in treated patients.

As described above, combination treatment may be preceded by a period of treatment with the CD73 antagonist antibody alone (“CD73 antibody monotherapy lead-in”). For example, one or more (e.g., 1-3 or 1-2) doses of the CD73 antagonist antibody may be administered as a monotherapy for 1-4 weeks (e.g., 1, 2, 3 or 4 weeks) or 1-3 weeks (e.g., 1, 2, or 3 weeks) prior to starting the combination treatment as Q1W or Q2W. In a particular embodiment, the CD73 antagonist antibody is administered as a monotherapy for 2 weeks prior to starting the combination treatment at Q1W or Q2W. In some embodiments, the lead-in phase is on a 2-week cycle. In certain embodiments, the lead-in phase is one 2-week cycle.

Suitable protocols for treating cancer (e.g., an advanced solid tumor) in a subject include, for example, administering to the subject a therapeutically effective amount of each of:

(a) a CD73 antagonist antibody comprising CDR1, CDR2 and CDR3 domains of the heavy chain variable region having the sequence set forth in SEQ ID NO: 6, and CDR1, CDR2 and CDR3 domains of the light chain variable region having the sequence set forth in SEQ ID NO: 7, and

(b) a PD-1 antagonist antibody comprising CDR1, CDR2 and CDR3 domains of the heavy chain variable region having the sequence set forth in SEQ ID NO: 18, and CDR1, CDR2 and CDR3 domains of the light chain variable region having the sequence set forth in SEQ ID NO: 19,

wherein one or more (e.g., 1-3, 1-2, 1, 2, 3) doses of the CD73 antagonist antibody are optionally administered within 1-3 weeks (e.g., 1, 2, or 3 weeks) prior to the first dose of the PD-1/PD-L1 axis antagonist antibody, e.g., for one 2-week cycle, in a “CD73 antibody monotherapy lead-in.”

In certain embodiments, the CD73 antagonist antibody comprising CDR1, CDR2 and CDR3 domains of the heavy chain variable region having the sequence set forth in SEQ ID NO: 6, and CDR1, CDR2 and CDR3 domains of the light chain variable region having the sequence set forth in SEQ ID NO: 7 is administered once a week at a fixed dose of about 100-1800 mg (e.g., 150-1600 mg, 150-1200 mg, 150-600 mg, 150-300 mg, 300-1600 mg, 300-1200 mg, 300-600 mg, 600 to 1200 mg, 600-1200 mg, 100 mg, 150 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, about 100 mg, about 150 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, or about 1600 mg), and the PD-1 antagonist antibody comprising CDR1, CDR2 and CDR3 domains of the heavy chain variable region having the sequence set forth in SEQ ID NO: 18, and CDR1, CDR2 and CDR3 domains of the light chain variable region having the sequence set forth in SEQ ID NO: 19 is administered once every two weeks at a fixed dose of 240 mg or about 240 mg.

In certain embodiments, the CD73 antagonist antibody comprising CDR1, CDR2 and CDR3 domains of the heavy chain variable region having the sequence set forth in SEQ ID NO: 6, and CDR1, CDR2 and CDR3 domains of the light chain variable region having the sequence set forth in SEQ ID NO: 7 is administered once a week at a fixed dose of about 100-1800 mg (e.g., 150-1600 mg, 150-1200 mg, 150-600 mg, 150-300 mg, 300-1600 mg, 300-1200 mg, 300-600 mg, 600 to 1200 mg, 600-1200 mg, 100 mg, 150 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, about 100 mg, about 150 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, or about 1600 mg), and the PD-1 antagonist antibody comprising CDR1, CDR2 and CDR3 domains of the heavy chain variable region having the sequence set forth in SEQ ID NO: 18, and CDR1, CDR2 and CDR3 domains of the light chain variable region having the sequence set forth in SEQ ID NO: 19 is administered once every four weeks at a fixed dose of 480 mg or about 480 mg.

In certain embodiments, the CD73 antagonist antibody comprising CDR1, CDR2 and CDR3 domains of the heavy chain variable region having the sequence set forth in SEQ ID NO: 6, and CDR1, CDR2 and CDR3 domains of the light chain variable region having the sequence set forth in SEQ ID NO: 7 is administered once every two weeks at a fixed dose of about 100-1800 mg (e.g., 150-1600 mg, 150-1200 mg, 150-600 mg, 150-300 mg, 300-1600 mg, 300-1200 mg, 300-600 mg, 600 to 1200 mg, 600-1200 mg, 100 mg, 150 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, about 100 mg, about 150 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, or about 1600 mg), and the PD-1 antagonist antibody comprising CDR1, CDR2 and CDR3 domains of the heavy chain variable region having the sequence set forth in SEQ ID NO: 18, and CDR1, CDR2 and CDR3 domains of the light chain variable region having the sequence set forth in SEQ ID NO: 19 is administered once every two weeks at a fixed dose of 240 mg or about 240 mg.

In certain embodiments, the CD73 antagonist antibody comprising CDR1, CDR2 and CDR3 domains of the heavy chain variable region having the sequence set forth in SEQ ID NO: 6, and CDR1, CDR2 and CDR3 domains of the light chain variable region having the sequence set forth in SEQ ID NO: 7 is administered once every two weeks at a fixed dose of about 100-1800 mg (e.g., 150-1600 mg, 150-1200 mg, 150-600 mg, 150-300 mg, 300-1600 mg, 300-1200 mg, 300-600 mg, 600 to 1200 mg, 600-1200 mg, 100 mg, 150 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, about 100 mg, about 150 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, or about 1600 mg) and the PD-1 antagonist antibody comprising CDR1, CDR2 and CDR3 domains of the heavy chain variable region having the sequence set forth in SEQ ID NO: 18, and CDR1, CDR2 and CDR3 domains of the light chain variable region having the sequence set forth in SEQ ID NO: 19 is administered once every four weeks at a fixed dose of 480 mg or about 480 mg.

In one embodiment, the CD73 antagonist antibody comprising CDR1, CDR2 and CDR3 domains of the heavy chain variable region having the sequence set forth in SEQ ID NO: 6, and CDR1, CDR2 and CDR3 domains of the light chain variable region having the sequence set forth in SEQ ID NO: 7 is administered once every two weeks at a fixed dose of 600 mg or about 600 mg, and the PD-1 antagonist antibody is administered once every two weeks at 240 mg or about 240 mg.

In one embodiment, the CD73 antagonist antibody comprising CDR1, CDR2 and CDR3 domains of the heavy chain variable region having the sequence set forth in SEQ ID NO: 6, and CDR1, CDR2 and CDR3 domains of the light chain variable region having the sequence set forth in SEQ ID NO: 7 is administered once every two weeks at a fixed dose of 600 mg or about 600 mg, and the PD-1 antagonist antibody is administered once every four weeks at 480 mg or about 480 mg.

In certain embodiments, the combination treatment with the CD73 antagonist antibody and PD-1 antagonist antibody comprises at least one administration cycle. In certain embodiments, the at least one administration cycle is a period of 28 days. In certain embodiments, the treatment consists of up to 6 cycles (i.e., 1, 2, 3, 4, 5, or 6 cycles). In certain embodiments, the treatment consists of up to 10 cycles (i.e., 1, 2, 3, 4, 5, 6, 7, 8, 9, or 10 cycles).

In certain embodiments, one or more (e.g., 1-3, 1-2, 1, 2, 3) doses of the CD73 antagonist antibody comprising CDR1, CDR2 and CDR3 domains of the heavy chain variable region having the sequence set forth in SEQ ID NO: 6, and CDR1, CDR2 and CDR3 domains of the light chain variable region having the sequence set forth in SEQ ID NO: 7, are administered within 1-3 weeks (e.g., 1, 2, or 3 weeks) prior to the first dose of the PD-1/PD-L1 axis antagonist antibody (e.g., nivolumab) (e.g., CD73 monotherapy lead-in). In some embodiments, the lead-in phase is on a 2-week cycle. In certain embodiments, the lead-in phase is one 2-week cycle or two 2-week cycles.

In certain embodiments, the CD73 antagonist antibody comprises heavy chain CDR1, CDR2, and CDR3 domains comprising the amino acid sequences set forth in SEQ ID NOs: 8, 9, and 10, respectively, and light chain CDR1, CDR2, and CDR3 domains comprising the amino acid sequences set forth in SEQ ID NOs: 11, 12, and 13, respectively. In certain embodiments, the CD73 antagonist antibody comprises heavy and light chain variable region sequences set forth in SEQ ID NOs: 6 and 7, respectively. In certain embodiments, the CD73 antagonist antibody comprises a heavy chain sequence set forth in SEQ ID NO: 3 or 4, and a light chain sequence set forth in SEQ ID NO: 5. In certain embodiments, the CD73 antagonist antibody comprises heavy and light chain variable region sequences that are at least 85%, 90%, 95%, 98%, or 99% identical with the heavy and light chain variable region sequences set forth in SEQ ID NOs: 6 and 7, respectively. In certain embodiments, the CD73 antagonist antibody comprises heavy and light chain sequences that are at least 85%, 90%, 95%, 98%, or 99% identical with the heavy chain sequence set forth in SEQ ID NO: 3 or 4, and the light chain sequence set forth in SEQ ID NO: 5.

In certain embodiments, the PD-1 antagonist antibody antagonist antibody comprises heavy chain CDR1, CDR2, and CDR3 domains comprising the amino acid sequences set forth in SEQ ID NOs: 20, 21, and 22, respectively, and light chain CDR1, CDR2, and CDR3 domains comprising the amino acid sequences set forth in SEQ ID NOs: 23, 24, and 25, respectively. In certain embodiments, the PD-1 antagonist antibody comprises heavy chain variable region sequences set forth in SEQ ID NOs: 18 and 19, respectively. In certain embodiments, the PD-1 antagonist antibody comprises a heavy chain sequence set forth in SEQ ID NO: 15 or 16, and a light chain sequence set forth in SEQ ID NO: 17. In certain embodiments, the PD-1 antagonist antibody comprises heavy and light chain variable region sequences that are at least 85%, 90%, 95%, 98%, or 99% identical with the heavy and light chain variable region sequences set forth in SEQ ID NOs: 18 and 19, respectively. In certain embodiments, the PD-1 antagonist antibody comprises heavy and light chain sequences that are at least 85%, 90%, 95%, 98%, or 99% identical with the heavy chain sequence set forth in SEQ ID NO: 15 or 16, and the light chain sequence set forth in SEQ ID NO: 17.

In certain embodiments, the CD73 antagonist antibody is CD73.A, and the PD-1 antagonist antibody is nivolumab, and the two antibodies are administered at one of the following combination doses: about 100-1800 mg of CD73.A Q1W and about 240-480 mg of nivolumab Q2W, about 100-1800 mg of CD73.A Q2W and about 240-480 mg of nivolumab Q2W, about 100-1800 mg of CD73.A Q1W and about 240-480 mg of nivolumab Q4W, about 100-1800 mg of CD73.A Q2W and about 240-480 mg of nivolumab Q4W, about 150 mg of CD73.A Q1W and about 240 mg of nivolumab Q2W; about 300 mg of CD73.A Q1W and about 240 mg of nivolumab Q2W; about 600 mg of CD73.A Q1W and about 240 mg of nivolumab Q2W; about 1200 mg of CD73.A Q1W and about 240 mg of nivolumab Q2W; about 1600 mg CD73.A Q1W and about 240 mg of nivolumab Q2W; about 150 mg of CD73.A Q1W and about 480 mg of nivolumab Q4W; about 300 mg of CD73.A Q1W and about 480 mg of nivolumab Q4W; about 600 mg of CD73.A Q1W and about 480 mg of nivolumab Q4W; about 1200 mg of CD73.A Q1W and about 480 mg of nivolumab Q4W; about 1600 mg of CD73.A Q1W and about 480 mg of nivolumab Q4W; about 150 mg of CD73.A Q2W and about 240 mg of nivolumab Q2W; about 300 mg of CD73.A Q2W and about 240 mg of nivolumab Q2W; about 600 mg of CD73.A Q2W and about 240 mg of nivolumab Q2W; about 1200 mg of CD73.A Q2W and about 240 mg of nivolumab Q2W; about 1600 mg of CD73.A Q2W and about 240 mg of nivolumab Q2W; about 150 mg of CD73.A Q2W and about 480 mg of nivolumab Q4W; about 300 mg of CD73.A Q2W and about 480 mg of nivolumab Q4W; about 600 mg of CD73.A Q2W and about 480 mg of nivolumab Q4W; about 1200 mg of CD73.A Q2W and about 480 mg of nivolumab Q4W; about 1600 mg of CD73.A Q2W and about 480 mg of nivolumab Q4W.

CD73 antagonist antibodies other than those comprising the CDR sequences described herein may also be used for treating cancer as described herein. For example, MEDI9447 or Phen 0203hIgG1, described in WO2016/075099, and CD73 antibodies described in WO2016/055609, WO2016/081748, and WO2017/152085) may be combined and/or administered and/or used as described herein.

PD-1/PD-L1 axis antagonists other than those comprising the CDR sequences of nivolumab may also be used for treating cancer as described herein. For example, pembrolizumab, avelumab, durvalumab, atezolizumab, or PDR-001 may be used.

In certain embodiments, the CD73 antagonist and/or PD-1/PD-L1 axis antagonist antibody are formulated for intravenous administration.

In certain embodiments, the CD73 antagonist antibody and/or PD-1/PD-L1 axis antagonist antibody are formulated for subcutaneous administration.

In certain embodiments, the CD73 antagonist antibody and the PD-1/PD-L1 axis antagonist antibody are administered on the same day at least once. In certain embodiments, when administered on the same day, the CD73 antagonist antibody and the PD-1/PD-L1 axis antagonist antibody are administered simultaneously at least once. In certain embodiments, when administered on the same day, the CD73 antagonist antibody and the PD-1/PD-L1 axis antagonist antibody are administered sequentially at least once.

In certain embodiments, steady state concentration of the CD73 antagonist antibody is achieved within 3-6 weeks (e.g., 3, 4, 5, or 6 weeks) of the first administration of the CD73 antagonist antibody, e.g., after initiation of combination treatment with the CD73 antagonist antibody and PD-1/PD-L1 axis antagonist antibody or CD73 antibody monotherapy lead-in.

In certain embodiments, target-mediated drug disposition (TMDD) saturation of the CD73 antagonist antibody is achieved, for example, when the antibody is administered at a fixed dose of about 600 mg or greater (e.g., about 600-1600 mg, e.g., 600-1400 mg, 600-1200 mg, 600-1000 mg, 600-800 mg, 800-1600 mg, 800-1400 mg, 800-1200 mg, 800-1000 mg, 1000-1600 mg, 1000-1400 mg, 1000-1200 mg, 1200-1600 mg, 1200-1400 mg, about 600 mg, about 700 mg, about 800 mg, about 900 mg, about 1000 mg, about 1100 mg, about 1200 mg, about 1300 mg, about 1400 mg, about 1500 mg, or about 1600 mg). In some embodiments, TMDD saturation is achieved within 3-6 weeks (e.g., 3, 4, 5, or 6 weeks) of the first administration of the CD73 antagonist antibody, e.g., after initiation of combination treatment with the CD73 antagonist antibody and PD-1/PD-L1 axis antagonist antibody or CD73 antibody monotherapy lead-in.

In certain embodiments, rapid target engagement is achieved following administration of the CD73 antagonist antibody. For example, at least 80%, at least 85%, at least 90%, at least 95%, 80-95%, 80-90%, 80-85%, 85-95%, 85-90%, or full receptor occupancy of the CD73 antagonist antibody is achieved immediately after the first administration of the CD73 antagonist antibody, e.g., after initiation of combination treatment with the CD73 antagonist antibody and PD-1/PD-L1 axis antagonist antibody or CD73 antibody monotherapy lead-in. In certain embodiments, at least 80%, at least 85%, at least 90%, at least 95%, 80-95%, 80-90%, 80-85%, 85-95%, 85-90%, or full receptor occupancy is achieved within 24 hours of the first administration of the CD73 antagonist antibody, e.g., after initiation of combination treatment with the CD73 antagonist antibody and PD-1/PD-L1 axis antagonist antibody or CD73 antibody monotherapy lead-in. In certain embodiments, at least 80%, at least 85%, at least 90%, at least 95%, 80-95%, 80-90%, 80-85%, 85-95%, 85-90%, or full receptor occupancy is achieved within 24 hours of the first administration of the CD73 antagonist antibody, e.g., after initiation of combination treatment with the CD73 antagonist antibody and PD-1/PD-L1 axis antagonist antibody or CD73 antibody monotherapy lead-in, for example, when the CD73 antagonist antibody is administered at a fixed dose of about 150 mg or greater (e.g., 150-1600 mg, e.g., 150-1400 mg, 150-1200 mg, 150-1000 mg, 150-800 mg, 150-600 mg, 150-400 mg, 150-300 mg, 150-200 mg, 300-1600 mg, 300-1400 mg, 300-1200 mg, 300-1000 mg, 300-800 mg, 300-600 mg, 600-1600 mg, 600-1400 mg, 600-1200 mg, 600-1000 mg, 600-800 mg, 800-1600 mg, 800-1400 mg, 800-1200 mg, 800-1000 mg, 1000-1600 mg, 1000-1400 mg, 1000-1200 mg, 1200-1600 mg, 1200-1400 mg, 150 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, or 1600 mg, about 150 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, or about 1600 mg). In certain embodiments, receptor occupancy is measured on peripheral B cells, such as CD19 B cells.

In certain embodiments, receptor occupancy of the CD73 antagonist antibody is sustained for a long period following administration of the CD73 antagonist antibody. For example, at least 80%, at least 85%, at least 90%, at least 95%, 80-95%, 80-90%, 80-85%, 85-95%, 85-90%, or full receptor occupancy of the CD73 antagonist antibody is sustained for at least 1-30 days, 15-30 days, 20-30 days, 30-60 days or longer after administration of the last dose of the CD73 antagonist antibody, e.g., after administration of the last dose of the combination treatment with the CD73 antagonist antibody and PD-1/PD-L1 axis antagonist antibody or CD73 antibody monotherapy lead-in, e.g., as measured on peripheral B cells (e.g., CD19 B cells), for example, when the CD73 antagonist antibody is administered at a fixed dose of about 150 mg or greater (e.g., 150-1600 mg, e.g., 150-1400 mg, 150-1200 mg, 150-1000 mg, 150-800 mg, 150-600 mg, 150-400 mg, 150-300 mg, 150-200 mg, 300-1600 mg, 300-1400 mg, 300-1200 mg, 300-1000 mg, 300-800 mg, 300-600 mg, 600-1600 mg, 600-1400 mg, 600-1200 mg, 600-1000 mg, 600-800 mg, 800-1600 mg, 800-1400 mg, 800-1200 mg, 800-1000 mg, 1000-1600 mg, 1000-1400 mg, 1000-1200 mg, 1200-1600 mg, 1200-1400 mg, 150 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, or 1600 mg, about 150 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, or about 1600 mg).

In certain embodiments, cell surface levels of CD73 rapidly decrease to undetectable levels after the first administration of the CD73 antagonist antibody, e.g., after initiation of combination treatment with the CD73 antagonist antibody and PD-1/PD-L1 axis antagonist antibody or CD73 antibody monotherapy lead-in. For example, cell surface levels of CD73 decrease to about 15%, about 10%, about 5%, about 5-15%, about 10-15%, about 5-10%, or 0% (undetectable) of baseline cell surface levels of CD73 within 12 hours, 16 hours, 20 hours, 24 hours, 12-16 hours, 12-20 hours, 12-24 hours, 16-20 hours, 16-24 hours, or 20-24 hours after the first administration of the CD73 antagonist antibody, e.g., after initiation of combination treatment with the CD73 antagonist antibody and PD-1/PD-L1 axis antagonist antibody or CD73 antibody monotherapy lead-in. In certain embodiments, cell surface levels of CD73 decrease to about 15%, about 10%, about 5%, about 5-15%, about 10-15%, about 5-10%, or 0% (undetectable) of baseline cell surface levels of CD73 within 12 hours, 16 hours, 20 hours, 24 hours, 12-16 hours, 12-20 hours, 12-24 hours, 16-20 hours, 16-24 hours, or 20-24 hours after the first administration of the CD73 antagonist antibody, e.g., after initiation of combination treatment with the CD73 antagonist antibody and PD-1/PD-L1 axis antagonist antibody or CD73 antibody monotherapy lead-in, for example, when the CD73 antagonist antibody is administered at a fixed dose of about 150 mg or greater (e.g., 150-1600 mg, e.g., 150-1400 mg, 150-1200 mg, 150-1000 mg, 150-800 mg, 150-600 mg, 150-400 mg, 150-300 mg, 150-200 mg, 300-1600 mg, 300-1400 mg, 300-1200 mg, 300-1000 mg, 300-800 mg, 300-600 mg, 600-1600 mg, 600-1400 mg, 600-1200 mg, 600-1000 mg, 600-800 mg, 800-1600 mg, 800-1400 mg, 800-1200 mg, 800-1000 mg, 1000-1600 mg, 1000-1400 mg, 1000-1200 mg, 1200-1600 mg, 1200-1400 mg, 150 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, or 1600 mg, about 150 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, or about 1600 mg). In certain embodiments, cell surface levels of CD73 are measured on peripheral B cells, such as CD19 B cells.

In certain embodiments, the combination treatment reduces cell surface levels of CD73 for a sustained period. For example, cell surface levels of CD73 remain at about 15%, about 10%, about 5%, about 5-15%, about 10-15%, about 5-10%, or 0% (undetectable) of baseline cell surface levels of CD73 for at least 1-30 days, 15-30 days, 20-30 days, 30-60 days or longer after administration of the last dose of the CD73 antagonist antibody, e.g., after administration of the last dose of the combination treatment with the CD73 antagonist antibody and PD-1/PD-L1 axis antagonist antibody or CD73 antibody monotherapy lead-in, e.g., as measured on peripheral B cells (e.g., CD19 B cells), for example, when the CD73 antagonist antibody is administered at a fixed dose of about 150 mg or greater (e.g., 150-1600 mg, e.g., 150-1400 mg, 150-1200 mg, 150-1000 mg, 150-800 mg, 150-600 mg, 150-400 mg, 150-300 mg, 150-200 mg, 300-1600 mg, 300-1400 mg, 300-1200 mg, 300-1000 mg, 300-800 mg, 300-600 mg, 600-1600 mg, 600-1400 mg, 600-1200 mg, 600-1000 mg, 600-800 mg, 800-1600 mg, 800-1400 mg, 800-1200 mg, 800-1000 mg, 1000-1600 mg, 1000-1400 mg, 1000-1200 mg, 1200-1600 mg, 1200-1400 mg, 150 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, or 1600 mg, about 150 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, or about 1600 mg).

In certain embodiments, levels of free soluble CD73 (sCD73) rapidly decrease to undetectable levels immediately after the first administration of the CD73 antagonist antibody, e.g., immediately after initiation of combination treatment with the CD73 antagonist antibody and PD-1/PD-L1 axis antagonist antibody or CD73 antibody monotherapy lead-in. For example, levels of sCD73 decrease to about 15%, about 10%, about 5%, about 5-15%, about 10-15%, about 5-10%, or 0% (undetectable) of baseline sCD73 levels within 2 hours, 4 hours, 6 hours, 8 hours, 2-8 hours, 2-6 hours, 2-4 hours, 4-8 hours, or 4-6 hours of the first administration of the CD73 antagonist antibody, e.g., after initiation of combination treatment with the CD73 antagonist antibody and PD-1/PD-L1 axis antagonist antibody or CD73 antibody monotherapy lead-in. In certain embodiments, levels of sCD73 decrease to about 15%, about 10%, about 5%, about 5-15%, about 10-15%, about 5-10%, or 0% (undetectable) of baseline levels of sCD73 within 2 hours, 4 hours, 6 hours, 8 hours, 2-8 hours, 2-6 hours, 2-4 hours, 4-8 hours, or 4-6 hours of the first administration of the CD73 antagonist antibody, e.g., after initiation of combination treatment with the CD73 antagonist antibody and PD-1/PD-L1 axis antagonist antibody or CD73 antibody monotherapy lead-in, for example, when the CD73 antagonist antibody is administered at a fixed dose of about 600 mg or greater (e.g., about 600-1600 mg, e.g., 600-1400 mg, 600-1200 mg, 600-1000 mg, 600-800 mg, 800-1600 mg, 800-1400 mg, 800-1200 mg, 800-1000 mg, 1000-1600 mg, 1000-1400 mg, 1000-1200 mg, 1200-1600 mg, 1200-1400 mg, 600 mg, 700 mg, 800 mg, 900 mg, 1000 mg, 1100 mg, 1200 mg, 1300 mg, 1400 mg, 1500 mg, 1600 mg, about 600 mg, about 700 mg, about 800 mg, about 900 mg, about 1000 mg, about 1100 mg, about 1200 mg, about 1300 mg, about 1400 mg, about 1500 mg, or about 1600 mg).

In certain embodiments, the combination treatment leads to sustained reduction of sCD73 levels. For example, levels of sCD73 remain at about 15%, about 10%, about 5%, about 5-15%, about 10-15%, about 5-10%, or 0% (undetectable) of baseline levels of sCD73 at least until the end of the last treatment cycle including the CD73 antagonist antibody (e.g., at least 1-30 days, 15-30 days, 20-30 days, 30-60 days or longer after the end of the last treatment cycle including the CD73 antagonist antibody), e.g., end of the last treatment cycle of the combination treatment with the CD73 antagonist antibody and PD-1/PD-L1 axis antagonist antibody or the end of the last treatment cycle of the CD73 antibody monotherapy lead-in, for example, when the CD73 antagonist antibody is administered at a fixed dose of about 600 mg or greater (e.g., about 600-1600 mg, e.g., 600-1400 mg, 600-1200 mg, 600-1000 mg, 600-800 mg, 800-1600 mg, 800-1400 mg, 800-1200 mg, 800-1000 mg, 1000-1600 mg, 1000-1400 mg, 1000-1200 mg, 1200-1600 mg, 1200-1400 mg, 600 mg, 700 mg, 800 mg, 900 mg, 1000 mg, 1100 mg, 1200 mg, 1300 mg, 1400 mg, 1500 mg, 1600 mg, about 600 mg, about 700 mg, about 800 mg, about 900 mg, about 1000 mg, about 1100 mg, about 1200 mg, about 1300 mg, about 1400 mg, about 1500 mg, or about 1600 mg).

sCD73 levels can be measured, for example, by obtaining a sample from a subject prior to, during, or both prior to and during treatment with a CD73 antagonist antibody (e.g., CD73.A), and contacting the sample with an agent that can detect soluble CD73, such as an anti-CD73 antibody, and determine the level of soluble CD73 in the blood or serum. In certain embodiments, the agent that detects the soluble CD73 antigen is not the antibody (or does not comprise the same variable regions) that was administered to the subject for the treatment.

In certain embodiments, CD73 enzyme activity is decreased in tumor cells and/or tumor vasculature after the first administration of the CD73 antagonist antibody, e.g., after initiation of combination treatment with the CD73 antagonist antibody and PD-1/PD-L1 axis antagonist antibody or CD73 antibody monotherapy lead-in, compared to before administration of the CD73 antagonist antibody. In certain embodiments, CD73 enzyme activity after the first administration of the CD73 antagonist antibody, e.g., after initiation of combination treatment with the CD73 antagonist antibody and PD-1/PD-L1 axis antagonist antibody or CD73 antibody monotherapy lead-in, is decreased in tumor cells and/or tumor vasculature compared to before administration of the CD73 antagonist antibody, for example, when the CD73 antagonist antibody is administered at a dose of about 150 mg or greater (e.g., 150-1600 mg, e.g., 150-1400 mg, 150-1200 mg, 150-1000 mg, 150-800 mg, 150-600 mg, 150-400 mg, 150-300 mg, 150-200 mg, 300-1600 mg, 300-1400 mg, 300-1200 mg, 300-1000 mg, 300-800 mg, 300-600 mg, 600-1600 mg, 600-1400 mg, 600-1200 mg, 600-1000 mg, 600-800 mg, 800-1600 mg, 800-1400 mg, 800-1200 mg, 800-1000 mg, 1000-1600 mg, 1000-1400 mg, 1000-1200 mg, 1200-1600 mg, 1200-1400 mg, 150 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, or 1600 mg, about 150 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, or about 1600 mg).

Methods for measuring serum concentrations of CD73 antagonist antibody, receptor occupancy of CD73 antagonist antibody, cell surface levels of CD73, CD73 internalization, levels of free soluble CD73, and CD73 enzyme activity are described in, e.g., WO2016/081748 and WO2017/152085, the contents of which are hereby incorporated by reference in their entireties.

In certain embodiments, the subject has received 1, 2, 3, or 4 or more prior therapies, e.g., systemic therapies. In certain embodiments, the subject has received one or more prior immunotherapies. In certain embodiments, the subject is refractory to the 1, 2, 3, or 4 or more systemic therapies or one or more previous immunotherapies. In one embodiment, the one or more previous immunotherapies includes a PD-1 or PD-L1 antagonist therapy, e.g., nivolumab.

In certain embodiments, the subject has progressed on or after prior cancer therapy, e.g., progressed on or after a previous immunotherapy. In some embodiments, the previous immunotherapy is a checkpoint inhibitor therapy, e.g., a PD-1 or PD-L1 antagonist therapy (e.g., nivolumab). In some embodiments, the previous immunotherapy is not a PD-1 or PD-L1 antagonist therapy.

In certain embodiments, the cancer is typically responsive to immunotherapy. In certain embodiments, the cancer is not typically responsive to immunotherapy, e.g., not typically responsive to an PD-1/PD-L1 axis antagonist (e.g., nivolumab).

In certain embodiments, combination treatment with the CD73 antagonist antibody and PD-1/PD-L1 axis antagonist antibody does not cause significant treatment-related adverse events, e.g., as determined in clinical trials.

In certain embodiments, the CD73 antagonist antibody and PD-1/PD-L1 axis antagonist antibody are administered to subjects who have a solid tumor associated with a cancer selected from the group consisting of: colorectal cancer, ovarian cancer, renal cell carcinoma, head and neck cancer, breast cancer, pancreatic cancer, prostate cancer, gastroesophageal cancer, hepatocellular carcinoma, melanoma, anal canal epidermoid carcinoma, endometrial cancer, gastric cancer, cervical cancer, gastroesophageal junction carcinoma, alveolar soft part carcinoma, cholangiocarcinoma, esophageal cancer, intrahepatic cholangiocarcinoma, leiomyosarcoma, Merkel cell carcinoma, squamous cell anorectal carcinoma, squamous cell carcinoma of the tongue, squamous cell carcinoma of the head and neck (SCCHN), and urothelial cancer. In certain embodiments, the patient to be treated has a biopsy-accessible lesion. In certain embodiments, the patient has a tumor that expresses CD73. In certain embodiments, the patient has a tumor that expresses high levels of CD73, e.g., higher levels of CD73 relative to the level of CD73 in healthy tissue of the same etiology as that of the tumor.

In certain embodiments, a subject has a cancer, e.g., a tumor, that is PD-L1 positive, e.g., has PD-L1 expression levels of ≥1%, ≥5% or ≥50%, as measured, e.g., with PD-L1 IHC 28-8 pharmDx assay. A subject may have a tumor with high PD-L1 expression, e.g., Tumor Proportion Score (TPS)≥50%. In certain embodiments, a subject has a tumor with TPS≥1%. TPS can be determined by FDA approved commercial kits.

Accordingly, in certain embodiments, the expression levels of PD-L1 are measured prior to initiation of treatment with the CD73 antagonist antibody, e.g., in combination with a PD-1/PD-L1 antagonist antibody. For example, in certain embodiments, the methods described herein comprise a step of first measuring the expression level of PD-L1 in the tumor of the subject with cancer, and if the expression level of PD-L1 is ≥1%, ≥5%, ≥10%, ≥25% or ≥50%, e.g., as measured with, e.g., the PD-L1 IHC 28-8 pharmDx assay, then the subject is treated with a therapeutically effective dose of the CD73 antagonist antibody, e.g., in combination with a PD-1/PD-L1 axis antagonist antibody.

Cancers to be treated with the combination of CD73 antagonist antibody (e.g., CD73.A) and a PD-1/PD-L1 axis antagonist antibody (e.g., nivolumab) can be metastatic cancers, refractory cancers (e.g., cancers refractory to previous immunotherapy, e.g., with a blocking CTLA-4 or PD-1 or PD-L1 antibody), and recurrent cancers.

In certain embodiments, the cancer is microsatellite stable. In certain embodiments, the cancer has a high tumor mutational burden (≥10 mutations/megabase, mut/mb) as determined, e.g., by FoundationOne CDx. In certain embodiments, the cancer has ≥1 or ≥5 mutations/megabase, mut/mb.

In one embodiment, the patient to be treated has pancreatic cancer. Accordingly, provided herein is a method of treating cancer, e.g., pancreatic cancer, in a human patient, the method comprising administering to the patient an effective amount of each of:

(a) a CD73 antagonist antibody comprising a heavy chain comprising the amino acid sequence set forth in SEQ ID NO: 3 or 4, and a light chain comprising the amino acid sequence set forth in SEQ ID NO: 5, and

(b) a PD-1 antagonist antibody comprising a heavy chain comprising the amino acid sequence set forth in SEQ ID NO: 15 or 16, and a light chain comprising the amino acid sequence set forth in SEQ ID NO: 17 (e.g., nivolumab),

wherein one or more (e.g., 1-3, 1-2, 1, 2, 3) doses of the CD73 antagonist antibody are administered within 1-3 weeks (e.g., 1, 2, or 3 weeks) prior to the first dose of the PD-1/PD-L1 axis antagonist antibody, e.g., for one 2-week cycle, in a “CD73 antibody monotherapy lead-in,”

wherein, following the CD73 antibody monotherapy lead-in, the CD73 antagonist antibody is administered once a week at a fixed dose of about 150-1600 mg (e.g., 150-1200 mg, 150-600 mg, 150-300 mg, 300-1600 mg, 300-1200 mg, 300-600 mg, 600 to 1200 mg, 600-1200 mg, 100 mg, 150 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, about 100 mg, about 150 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, or about 1600 mg) in combination with the PD-1 antagonist antibody, which is administered once every two weeks at a fixed dose of 240 mg or about 240 mg or once every four weeks at a fixed dose of 480 mg or about 480 mg, wherein the combination treatment consists, e.g., of up to six 28-day cycles. In certain embodiments, the patient has received (e.g., and progressed on) one or more prior therapies or one or more prior immunotherapies to treat the cancer.

In one embodiment, provided herein is a method of treating cancer, e.g., pancreatic cancer, in a human patient, the method comprising administering to the patient an effective amount of each of:

(a) a CD73 antagonist antibody comprising a heavy chain comprising the amino acid sequence set forth in SEQ ID NO: 3 or 4, and a light chain comprising the amino acid sequence set forth in SEQ ID NO: 5, and

(b) a PD-1 antagonist antibody comprising a heavy chain comprising the amino acid sequence set forth in SEQ ID NO: 15 or 16, and a light chain comprising the amino acid sequence set forth in SEQ ID NO: 17 (e.g., nivolumab),

wherein the CD73 antagonist antibody is administered once a week at a fixed dose of about 150-1600 mg (e.g., 150-1200 mg, 150-600 mg, 150-300 mg, 300-1600 mg, 300-1200 mg, 300-600 mg, 600 to 1200 mg, 600-1200 mg, 100 mg, 150 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, about 100 mg, about 150 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, about 1500 mg, or about 1600 mg) in combination with the PD-1 antagonist antibody, which is administered once every two weeks at a fixed dose of 240 mg or about 240 mg or once every four weeks at a fixed dose of 480 mg or about 480 mg, wherein the combination treatment consists, e.g., of up to six 28-day cycles. In certain embodiments, the patient has received (e.g., and progressed on) one or more prior therapies or one or more prior immunotherapies to treat the cancer.

In one embodiment, provided herein is a method of treating cancer, e.g., pancreatic cancer, in a human patient, the method comprising administering to the patient an effective amount of each of:

(a) a CD73 antagonist antibody comprising a heavy chain comprising the amino acid sequence set forth in SEQ ID NO: 3 or 4, and a light chain comprising the amino acid sequence set forth in SEQ ID NO: 5, and

(b) a PD-1 antagonist antibody comprising a heavy chain comprising the amino acid sequence set forth in SEQ ID NO: 15 or 16, and a light chain comprising the amino acid sequence set forth in SEQ ID NO: 17 (e.g., nivolumab),

wherein one or more (e.g., 1-3, 1-2, 1, 2, 3) doses of the CD73 antagonist antibody are administered within 1-3 weeks (e.g., 1, 2, or 3 weeks) prior to the first dose of the PD-1/PD-L1 axis antagonist antibody, e.g., for one 2-week cycle, in a “CD73 antibody monotherapy lead-in,”

wherein, following the CD73 antibody monotherapy lead-in, the CD73 antagonist antibody is administered once every two weeks at a fixed dose of 600 mg or about 600 mg in combination with the PD-1 antagonist antibody which is administered once every two weeks at 240 mg or about 240 mg or once every four weeks at a fixed dose of 480 mg or about 480 mg, wherein the combination therapy consists, e.g., of up to six 28-day cycles. In certain embodiments, the patient has received (e.g., and progressed on) one or more prior therapies or one or more prior immunotherapies to treat the cancer.

In one embodiment, provided herein is a method of treating cancer, e.g., pancreatic cancer, in a human patient, the method comprising administering to the patient an effective amount of each of:

(a) a CD73 antagonist antibody comprising a heavy chain comprising the amino acid sequence set forth in SEQ ID NO: 3 or 4, and a light chain comprising the amino acid sequence set forth in SEQ ID NO: 5, and

(b) a PD-1 antagonist antibody comprising a heavy chain comprising the amino acid sequence set forth in SEQ ID NO: 15 or 16, and a light chain comprising the amino acid sequence set forth in SEQ ID NO: 17 (e.g., nivolumab),

wherein the CD73 antagonist antibody is administered once every two weeks at a fixed dose of 600 mg or about 600 mg in combination with the PD-1 antagonist antibody, which is administered once every two weeks at 240 mg or about 240 mg or once every four weeks at a fixed dose of 480 mg or about 480 mg, wherein the combination treatment consists, e.g., of up to six 28-day cycles. In certain embodiments, the patient has received (e.g., and progressed on) one or more prior therapies or one or more prior immunotherapies to treat the cancer.

In certain embodiments, the methods described above result in one or more (e.g., 1, 2, 3, 4, 5, 6, 7, or 8) of the following:

(a) steady state serum concentration of the CD73 antagonist antibody is achieved 3, 4, 5, or 6 weeks after the first administration of the CD73 antagonist antibody;

(b) full receptor occupancy of the CD73 antagonist antibody, e.g., on peripheral B cells such as CD19 B cells, is achieved within 24 hours of the first administration of the CD73 antagonist antibody;

(c) full receptor occupancy of the CD73 antagonist antibody is sustained for at least 30 days after administration of the last dose of the CD73 antagonist antibody;

(d) undetectable cell surface levels of CD73 on peripheral B cells, e.g., CD19 B cells within 24 hours of the first administration of the CD73 antagonist antibody;

(e) undetectable cell surface levels of CD73 up to at least 30 days after administration of the last dose of the CD73 antagonist antibody;

(f) undetectable free soluble CD73 within 6 hours of the first administration of the CD73 antagonist antibody;

(g) undetectable free soluble CD73 at the end of the last treatment cycle including the CD73 antagonist antibody; and

(h) decrease of CD73 enzyme activity in tumor cells and/or tumor vasculature compared to before administration of the CD73 antagonist antibody.

A patient receiving a treatment described herein may be a patient who has one or more of the inclusion criteria set forth in Example 1, or who does not have one or more of the exclusion criteria set forth in Example 1.

Additional cancers which can be treated using a CD73 antagonist antibody, e.g., in combination with a PD-1/PD-L1 axis antagonist antibody, include, for example, squamous cell carcinoma, small-cell lung cancer, non-small cell lung cancer, squamous non-small cell lung cancer (NSCLC), non NSCLC, glioma, gastrointestinal cancer, renal cancer (e.g. clear cell carcinoma), liver cancer, kidney cancer, thyroid cancer, neuroblastoma, glioblastoma (glioblastoma multiforme), stomach cancer, bladder cancer, hepatoma, colon carcinoma, germ cell tumor, pediatric sarcoma, sinonasal natural killer, melanoma (e.g., metastatic malignant melanoma, such as cutaneous or intraocular malignant melanoma), bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally-induced cancers including those induced by asbestos, virus-related cancers (e.g., human papilloma virus (HPV)-related tumor), and hematologic malignancies derived from either of the two major blood cell lineages, i.e., the myeloid cell line (which produces granulocytes, erythrocytes, thrombocytes, macrophages and mast cells) or lymphoid cell line (which produces B, T, NK and plasma cells), such as all types of luekemias, lymphomas, and myelomas, e.g., acute, chronic, lymphocytic and/or myelogenous leukemias, such as acute leukemia (ALL), acute myelogenous leukemia (AML), chronic lymphocytic leukemia (CLL), and chronic myelogenous leukemia (CML), undifferentiated AML (M0), myeloblastic leukemia (M1), myeloblastic leukemia (M2; with cell maturation), promyelocytic leukemia (M3 or M3 variant [M3V]), myelomonocytic leukemia (M4 or M4 variant with eosinophilia [M4E]), monocytic leukemia (M5), erythroleukemia (M6), megakaryoblastic leukemia (M7), isolated granulocytic sarcoma, and chloroma; lymphomas, such as Hodgkin's lymphoma (HL), non-Hodgkin's lymphoma (NHL), B-cell lymphomas, T-cell lymphomas, lymphoplasmacytoid lymphoma, monocytoid B-cell lymphoma, mucosa-associated lymphoid tissue (MALT) lymphoma, anaplastic (e.g., Ki 1+) large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic; and lymphoma/leukaemia (T-Lbly/T-ALL), peripheral T-cell lymphoma, lymphoblastic lymphoma, post-transplantation lymphoproliferative disorder, true histiocytic lymphoma, primary central nervous system lymphoma, primary effusion lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T-cell lymphoma (CTLC) (also called mycosis fungoides or Sezary syndrome), and lymphoplasmacytoid lymphoma (LPL) with Waldenstrom's macroglobulinemia; myelomas, such as IgG myeloma, light chain myeloma, nonsecretory myeloma, smoldering myeloma (also called indolent myeloma), solitary plasmocytoma, and multiple myelomas, chronic lymphocytic leukemia (CLL), hairy cell lymphoma; hematopoietic tumors of myeloid lineage, tumors of mesenchymal origin, including fibrosarcoma and rhabdomyoscarcoma; seminoma, teratocarcinoma, tumors of the central and peripheral nervous, including astrocytoma, schwannomas; tumors of mesenchymal origin, including fibrosarcoma, rhabdomyoscaroma, and osteosarcoma; and other tumors, including xeroderma pigmentosum, keratoacanthoma, seminoma, thyroid follicular cancer and teratocarcinoma, hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) preferably of the T-cell type; a/d T-NHL hepatosplenic lymphoma; peripheral/post-thymic T cell lymphoma (pleomorphic and immunoblastic subtypes); angiocentric (nasal) T-cell lymphoma; cancer of the head or neck, renal cancer, rectal cancer, cancer of the thyroid gland; acute myeloid lymphoma, as well as any combinations of said cancers.

In certain embodiments, the patient has a tumor that expresses CD73 and tumor infiltrating lymphocytes (TILs) in the tumor that express PD-1. In certain embodiments, the patient has a tumor that expresses high levels of CD73 and TILs that express high levels of PD-1.

In certain embodiments, the patient has a tumor that expresses CD73 and A2A adenosine receptor (A2AR). In certain embodiments, the patient has a tumor that expresses CD73 and A2AR and TILs that express PD-1. In certain embodiments, the patient has a tumor that expresses high levels of CD73 and A2AR and TILs that express high levels of PD-1.

Levels of expression of CD73 and A2AR in tumors, and PD-1 in TILs can be determined using standard methods in the art, e.g., immunohistochemistry or quantification of mRNA levels.

In certain embodiments, the treatment produces at least one therapeutic effect chosen from a reduction in size of a tumor, reduction in number of metastatic lesions over time, complete response, partial response, and stable disease.

With respect to target lesions, responses to therapy may include:

| | |
|---|---|
| Complete Response (CR) | Disappearance of all target lesions. Any |
| (RECIST V1.1) | pathological lymph nodes (whether target |
| | or non-target) must have reduction in |
| | short axis to <10 mm. |
| Partial Response (PR) | At least a 30% decrease in the sum of the |
| (RECIST V1.1) | diameters of target lesions, taking as |
| | reference the baseline sum diameters. |
| Progressive Disease (PD) | At least a 20% increase in the sum of the |
| (RECIST V1.1) | diameters of target lesions, taking as |
| | reference the smallest sum on study (this |
| | includes the baseline sum if that is the |
| | smallest on study). In addition to the |
| | relative increase of 20%, the sum must |
| | also demonstrate an absolute increase of at |
| | least 5 mm. (Note: the appearance of one |
| | or more new lesions is also considered |
| | progression). |
| Stable Disease (SD) | Neither sufficient shrinkage to qualify for |
| (RECIST V1.1) | PR nor sufficient increase to qualify for |
| | PD, taking as reference the smallest sum |
| | diameters while on study. |
| Immune-related | Disappearance of all target lesions. Any |
| Complete Response | pathological lymph nodes (whether target |
| (irCR) | or non-target) must have reduction in |
| (irRECIST) | short axis to <10 mm. |
| Immune-related Partial | At least a 30% decrease in the sum of |
| Response (irPR) | diameters of target lesions and all new |
| (irRECIST) | measurable lesions (ie Percentage Change |
| | in Tumor Burden), taking as reference the |
| | baseline sum diameters. Note: the |
| | appearance of new measurable lesions is |
| | factored into the overall Tumor Burden, |
| | but does not automatically qualify as |
| | progressive disease until the sum of the |
| | diameters increases by ≥20% when |
| | compared to nadir. |
| Immune-related | At least a 20% increase in Tumor Burden |
| Progressive Disease | (ie the sum of diameters of target lesions, |
| (irPD) | and any new measurable lesions) taking as |
| (irRECIST) | reference the smallest sum on study (this |
| | includes the baseline sum if that is the |
| | smallest on study). In addition to the |
| | relative increase of 20%, the sum must |
| | also demonstrate an absolute increase of at |
| | least 5 mm. Tumor assessments using |
| | immune-related criteria for progressive |
| | disease incorporates the contribution of |
| | new measurable lesions. Each net |
| | percentage change in tumor burden per |
| | assessment accounts for the size and |
| | growth kinetics of both old and new |
| | lesions as they appear. |
| Immune-related Stable | Neither sufficient shrinkage to qualify for |
| Disease (irSD) | irPR nor sufficient increase to qualify for |
| (irRECIST) | irPD, taking as reference the smallest sum |
| | diameters while on study. |

With respect to non-target lesions, responses to therapy may include:

| | |
|---|---|
| Complete Response (CR) | Disappearance of all non-target lesions. |
| (RECIST V1.1) | All lymph nodes must be non-pathological |
| | in size (<10 mm short axis). |
| Non-CR/Non-PD | Persistence of one or more non-target |
| (RECIST V1.1) | lesion(s). |
| Progressive Disease (PD) | Unequivocal progression of existing non- |
| (RECIST V1.1) | target lesions. The appearance of one or |
| | more new lesions is also considered |
| | progression. |
| Immune-related | Disappearance of all non-target lesions. |
| Complete Response | All lymph nodes must be non-pathological in |
| (irCR) | size (<10 mm short axis). |
| (irRECIST) | |
| Immune-related | Increases in number or size of non-target |
| Progressive Disease | lesion(s) does not constitute progressive |
| (irPD) | disease unless/until Tumor Burden |
| (irRECIST) | increases by 20% (ie the sum of the |
| | diameters at nadir of target lesions and |
| | any new measurable lesions increases by |
| | the required amount). Non-target lesions |
| | are not considered in the definition of |
| | Stable Disease and Partial Response. |

Patients treated according to the methods disclosed herein preferably experience improvement in at least one sign of cancer. In certain embodiments, improvement is measured by a reduction in the quantity and/or size of measurable tumor lesions. In certain embodiments, lesions can be measured on chest x-rays or CT or MRI films. In certain embodiments, cytology or histology can be used to evaluate responsiveness to a therapy.

In certain embodiments, the patient treated exhibits a complete response (CR), a partial response (PR), stable disease (SD), immune-related complete disease (irCR), immune-related partial response (irPR), or immune-related stable disease (irSD). In certain embodiments, the patient treated experiences tumor shrinkage and/or decrease in growth rate, i.e., suppression of tumor growth. In certain embodiments, unwanted cell proliferation is reduced or inhibited. In certain embodiments, one or more of the following can occur: the number of cancer cells can be reduced; tumor size can be reduced; cancer cell infiltration into peripheral organs can be inhibited, retarded, slowed, or stopped; tumor metastasis can be slowed or inhibited; tumor growth can be inhibited; recurrence of tumor can be prevented or delayed; one or more of the symptoms associated with cancer can be relieved to some extent.

In certain embodiments, administration of effective amounts of the CD73 antagonist antibody (e.g., CD73.A) and PD-1/PD-L1 axis antagonist antibody (e.g., nivolumab) according to any of the methods provided herein produces at least one therapeutic effect selected from the group consisting of reduction in size of a tumor, reduction in number of metastatic lesions appearing over time, complete remission, partial remission, or stable disease. In certain embodiments, the methods of treatment produce a comparable clinical benefit rate (CBR=CR+PR+SD≥6 months) better than that achieved by a CD73 antagonist antibody or PD-1/PD-L1 axis antagonist antibody alone. In certain embodiments, the improvement of clinical benefit rate is about 20% 20%, 30%, 40%, 50%, 60%, 70%, 80% or more compared to the CD73 antagonist antibody or PD-1/PD-L1 axis antagonist antibody alone.

In certain embodiments, disease assessment before, during, and/or after treatment is performed by computed tomography and/or magnetic resonance imaging. In certain embodiments, disease assessment is performed at baseline and every 7-10 weeks from the start of treatment for until treatment discontinuation or completion.

In certain embodiments, anti-tumor efficacy is measured by ORR, DOR, and PFSR. ORR is defined herein as the proportion of all treated patients whose best overall response (BOR) is either a CR or PR. BOR is defined herein as the best response designation over the study as a whole, recorded between the dates of first dose until the last tumor assessment prior to subsequent therapy. DOR is defined herein as the time between the date of first response and the date of disease progression or death, whichever occurs first. PFSR is defined herein as the proportion of treated subjects remaining progression free and surviving. For example, PFSR at 24 weeks refers to the proportion of treated subjects remaining progression free and surviving at 24 weeks.

In certain embodiments, disease assessment before, during, and/or after treatment is performed on a biopsy sample obtained from the patient. The biopsy sample can be, e.g., a core-needle, excisional, or incisional biopsy.

In certain embodiments, the patient to be treated has at least one lesion with measurable disease as defined by RECIST v1.1.

In certain embodiments, the patient to be treated has progressive disease, as defined by RECIST v1.1.

In certain embodiments, the patient to be treated has a malignancy that is advanced (e.g., metastatic and/or unresectable) with measurable disease, as defined by RECIST v1.1.

In certain embodiments, the patient to be treated has received, and then progressed or been intolerant to, at least 1 standard treatment regimen in the advanced or metastatic setting.

In certain embodiments, the patient to be treated has been previously treated with an agent specifically targeting checkpoint pathway inhibition (e.g., anti-PD-1, anti-PD-L1, anti-PD-L2, anti-LAG-3, and anti-CTLA-4 antibody).

In certain embodiments, the patient to be treated has been previously treated with an agent specifically targeting T-cell co-stimulation pathways (e.g., anti-glucocorticoid induced tumor necrosis factor receptor, anti-CD137, and anti-OX40 antibody).

In certain embodiments, the patient to be treated has undergone prior palliative radiotherapy.

In certain embodiments, the patient to be treated has adequate organ function, as summarized by the following: white blood cell count ≥2000/μL, neutrophils ≥1500/μL, platelets ≥100×10³/μL, hemoglobin ≥9 g/dL, alanine aminotransferase (ALT) and aspartate aminotransferase (AST)≤3× the upper limit of normal (ULN), total bilirubin ≤1.5×ULN, albumin >2 g/dL (20 g/L), International normalized ratio <1.5×ULN, activated partial thromboplastin time <1.5×ULN, clinically normal thyroid function or have controlled hypothyroidism on appropriate thyroid supplementation, and serum creatinine ≤1.5×ULN or creatinine clearance (CrCl)≥40 mL/min.

In certain embodiments, the patient to be treated does not have known or suspected CNS metastases, untreated CNS metastases, or with the CNS as the only site of disease. However, in certain embodiments, patients with controlled brain metastases, defined as no radiographic progression for at least 4 weeks following radiation and/or surgical treatment (or 4 weeks of observation if no intervention is clinically indicated), off of steroids for at least 2 weeks, and no new or progressive neurological signs and symptoms, are amenable to treatment with the methods disclosed herein.

In certain embodiments, the patient to be treated does not have carcinomatous meningitis.

In certain embodiments, the patient to be treated does not have clinically relevant ascites (i.e., ascities requiring paracentesis) or moderate radiographic ascites.

In certain embodiments, the patient to be treated has not been previously treated with nivolumab.

In certain embodiments, the patient to be treated does not have a prior malignancy.

In certain embodiments, the patient to be treated does not have a different active malignancy requiring concurrent intervention.

In certain embodiments, the patient to be treated does not have a prior organ allograft.

In certain embodiments, the patient to be treated has not been previously treated with an anti-CD73 antibody, an anti-CD39 antibody, or an adenosine 2A receptor inhibitor.

In certain embodiments, the patient to be treated does not have a prior history of cerebrovascular accident, deep vein thrombosis, or other arterial thrombus.

In certain embodiments, the patient to be treated does not have active, known, or suspected autoimmune disease. However, in certain embodiments, patients with vitiligo, Type 1 diabetes mellitus, residual hypothyroidism due to autoimmune condition only requiring hormone replacement, patients with euthyroid with a history of Grave's disease, psoriasis not requiring systemic treatment, or conditions not expected to recur in the absence of an external trigger are amenable to treatment with the methods disclosed herein.

In certain embodiments, the patient to be treated does not have interstitial lung disease that is symptomatic or may interfere with the detection or management of suspected drug-related pulmonary toxicity.

In certain embodiments, the patient to be treated does not have chronic obstructive pulmonary disease requiring recurrent steroids bursts or chronic steroids at doses greater than 10 mg/day of prednisone or the equivalent.

In certain embodiments, the patient to be treated does not have a condition that requires systemic treatment with either corticosteroids (>10 mg daily prednisone equivalents) or other immunosuppressive medications within 14 days of study drug administration, except for adrenal replacement steroid doses >10 mg daily prednisone equivalent in the absence of active autoimmune disease.

In certain embodiments, the patient to be treated does not have uncontrolled or significant cardiovascular disease including, e.g., myocardial infarction or stroke/transient ischemic attack within 6 months of the initiation of treatment, uncontrolled angina within 3 months of the initiation of treatment, a history of clinically significant arrhythmias (e.g., ventricular tachycardia, ventricular fibrillation, or torsades de pointes), QT interval corrected for heart rate using Fridericia's formula (QTcF) prolongation >480 msec, history of other clinically significant heart disease (e.g., cardiomyopathy, congestive heart failure with New York Heart Association [NYHA] functional Classification III to IV, pericarditis, significant pericardial effusion), a requirement for daily supplemental oxygen therapy,

In certain embodiments, the patient to be treated does not have active hepatitis.

In certain embodiments, the patient to be treated does not have active bacterial, viral, or fungal infections ≤7 days prior to initiation of treatment.

In certain embodiments, the patient to be treated does not have a history of testing positive for human immunodeficiency virus (HIV) or known acquired immunodeficiency syndrome (AIDS).

In certain embodiments, the patient to be treated does not have evidence or history of active or latent tuberculosis infection.

In certain embodiments, the patient to be treated has not undergone major surgery within 4 weeks of treatment.

In certain embodiments, all toxicities attributed to prior anti-cancer therapy other than alopecia and fatigue in the patient is resolved to Grade 1 (National Cancer Institute [NCI] Common Terminology Criteria for Adverse Events [CTCAE] Version 4.03) or baseline prior to initiation of treatment. However, in certain embodiments, those with toxicities attributed to prior anti-cancer therapy that are not expected to resolve and result in long-lasting sequelae, such as chronic neuropathy after platinum based therapy, are amenable to treatment with the methods disclosed herein.

In certain embodiments, the patient to be treated has not used non-oncology vaccines containing live virus for prevention of infectious diseases within 12 weeks of treatment.

In certain embodiments, the patient to be treated has not used packed red blood cells or received a platelet transfusion within 2 weeks prior to treatment.

In certain embodiments, the patient to be treated does not have a history of allergy to nivolumab.

In certain embodiments, the patient to be treated does not have a history of drug allergy (such as anaphylaxis) to prior anti-cancer immune modulating therapies (e.g., checkpoint inhibitors, T-cell co-stimulatory antibodies).

### Combination Therapies

Antibodies to CD73, e.g., the CD73 antagonist antibodies described herein, e.g., in combination with a PD-1/PD-L1 axis antagonist antibody (e.g., nivolumab), can be combined with an immunogenic agent, such as cancerous cells, purified tumor antigens (including recombinant proteins, peptides, and carbohydrate molecules), cells, and cells transfected with genes encoding immune stimulating cytokines (He et al (2004) J. Immunol. 173:4919-28). Non-limiting examples of tumor vaccines that can be used include peptides of melanoma antigens, such as peptides of gp100, MAGE antigens, Trp-2, MART1 and/or tyrosinase, or tumor cells transfected to express the cytokine GM-CSF (discussed further below).

In humans, some tumors have been shown to be immunogenic such as melanomas. By lowering the threshold of T cell activation via CD73 inhibition, the tumor responses in the host can be activated, allowing treatment of non-immunogenic tumors or those having limited immunogenicity.

The CD73 antagonist antibodies described herein, e.g., in combination with a PD-1/PD-L1 axis antagonist antibody, may be combined with a vaccination protocol. Many experimental strategies for vaccination against tumors have been devised (see Rosenberg, S., 2000, Development of Cancer Vaccines, ASCO Educational Book Spring: 60-62; Logothetis, C., 2000, ASCO Educational Book Spring: 300-302; Khayat, D. 2000, ASCO Educational Book Spring: 414-428; Foon, K. 2000, ASCO Educational Book Spring: 730-738; see also Restifo, N. and Sznol, M., Cancer Vaccines, Ch. 61, pp. 3023-3043 in DeVita et al. (eds.), 1997, Cancer: Principles and Practice of Oncology, Fifth Edition). In one of these strategies, a vaccine is prepared using autologous or allogeneic tumor cells. These cellular vaccines have been shown to be most effective when the tumor cells are transduced to express GM-CSF. GM-CSF has been shown to be a potent activator of antigen presentation for tumor vaccination (Dranoff et al. (1993) Proc. Natl. Acad. Sci U.S.A. 90: 3539-43).

The study of gene expression and large scale gene expression patterns in various tumors has led to the definition of so called tumor specific antigens (Rosenberg, S A (1999) Immunity 10: 281-7). In many cases, these tumor specific antigens are differentiation antigens expressed in the tumors and in the cell from which the tumor arose, for example melanocyte antigens gp100, MAGE antigens, and Trp-2. More importantly, many of these antigens can be shown to be the targets of tumor specific T cells found in the host. CD73 inhibition can be used in conjunction with a collection of recombinant proteins and/or peptides expressed in a tumor in order to generate an immune response to these proteins. These proteins are normally viewed by the immune system as self antigens and are therefore tolerant to them. The tumor antigen can include the protein telomerase, which is required for the synthesis of telomeres of chromosomes and which is expressed in more than 85% of human cancers and in only a limited number of somatic tissues (Kim et al. (1994) Science 266: 2011-2013). Tumor antigen can also be “neo-antigens” expressed in cancer cells because of somatic mutations that alter protein sequence or create fusion proteins between two unrelated sequences (i.e., bcr-abl in the Philadelphia chromosome), or idiotype from B cell tumors.

Other tumor vaccines can include the proteins from viruses implicated in human cancers such a Human Papilloma Viruses (HPV), Hepatitis Viruses (HBV and HCV) and Kaposi's Herpes Sarcoma Virus (KHSV). Another form of tumor specific antigen which can be used in conjunction with CD73 inhibition is purified heat shock proteins (HSP) isolated from the tumor tissue itself. These heat shock proteins contain fragments of proteins from the tumor cells and these HSPs are highly efficient at delivery to antigen presenting cells for eliciting tumor immunity (Suot & Srivastava (1995) Science 269:1585-1588; Tamura et al. (1997) Science 278:117-120).

Dendritic cells (DC) are potent antigen presenting cells that can be used to prime antigen-specific responses. DC's can be produced ex vivo and loaded with various protein and peptide antigens as well as tumor cell extracts (Nestle et al. (1998) Nature Medicine 4: 328-332). DCs can also be transduced by genetic means to express these tumor antigens as well. DCs have also been fused directly to tumor cells for the purposes of immunization (Kugler et al. (2000) Nature Medicine 6:332-336). As a method of vaccination, DC immunization can be effectively combined with CD73 inhibition to activate more potent anti-tumor responses.

The CD73 antagonist antibodies described herein, e.g., in combination with a PD-1/PD-L1 axis antagonist antibody, can be combined with standard cancer treatments (e.g., surgery, radiation, and chemotherapy). CD73 inhibition can be effectively combined with chemotherapeutic regimes. In these instances, it may be possible to reduce the dose of chemotherapeutic reagent administered (Mokyr et al. (1998) Cancer Research 58: 5301-5304). An example of such a combination is a CD73 antagonist antibody in combination with decarbazine for the treatment of melanoma. Another example of such a combination is a CD73 antagonist antibody in combination with interleukin-2 (IL-2) for the treatment of melanoma. The scientific rationale behind the combined use of CD73 inhibition and chemotherapy is that cell death, that is a consequence of the cytotoxic action of most chemotherapeutic compounds, should result in increased levels of tumor antigen in the antigen presentation pathway. Other combination therapies that may result in synergy with CD73 inhibition through cell death are radiation, surgery, and hormone deprivation.

Each of these protocols creates a source of tumor antigen in the host. Angiogenesis inhibitors can also be combined with CD73 inhibition. Inhibition of angiogenesis leads to tumor cell death which may feed tumor antigen into host antigen presentation pathways.

Yet another example of such a combination is a CD73 antagonist antibody described herein, e.g., in combination with a PD-1/PD-L1 axis antagonist antibody, in combination with an anti-CD39, anti-A2AR or chemical inhibitor (e.g., SCH58261), or antiA2BR antibody or chemical inhibitor. The scientific rationale behind the combined use of CD73 inhibition and inhibition of CD39, A2AR, or A2BR is that these proteins are also linked to CD73 biological function and signaling. Specifically, CD39 catalyzes the conversion of ATP or ADP to AMP, thus providing the substrate (AMP) for CD73 enzymatic activity (i.e. the conversion of AMP to adenosine). Furthermore, adenosine is a ligand for four known receptors, including A1R, A2AR, A2BR, and A3. A2AR and A2BR have been shown to regulate tumor cell proliferation, growth, migration, and metastasis, as well as T-cell activation in the tumor environment through cAMP signaling.

The CD73 antagonist antibodies described herein, e.g., in combination with a PD-1/PD-L1 axis antagonist antibody, can also be used in combination with bispecific antibodies that target Fcα or Fcγ receptor-expressing effectors cells to tumor cells (see, e.g., U.S. Pat. Nos. 5,922,845 and 5,837,243). Bispecific antibodies can be used to target two separate antigens. For example anti-Fc receptor/anti tumor antigen (e.g., Her-2/neu) bispecific antibodies have been used to target macrophages to sites of tumor. This targeting may more effectively activate tumor specific responses. Alternatively, antigen may be delivered directly to DCs by the use of bispecific antibodies which bind to tumor antigen and a dendritic cell specific cell surface marker.

Tumors evade host immune surveillance by a large variety of mechanisms. Many of these mechanisms may be overcome by the inactivation of proteins which are expressed by the tumors and which are immunosuppressive. These include among others TGF-β (Kehrl et al. (1986) J. Exp. Med. 163: 1037-1050), IL-10 (Howard & O'Garra (1992) Immunology Today 13: 198-200), and Fas ligand (Hahne et al. (1996) Science 274: 1363-1365). Antibodies to each of these entities can be used in combination with the CD73 antagonist antibodies described herein, e.g., in combination with a PD-1/PD-L1 axis antagonist antibody, to counteract the effects of the immunosuppressive agent and favor tumor immune responses by the host.

Other antibodies which activate host immune responsiveness can be used in combination with the CD73 antagonist antibodies described herein, e.g., in combination with a PD-1/PD-L1 axis antagonist antibody. These include molecules on the surface of dendritic cells which activate DC function and antigen presentation. Anti-CD40 antibodies are able to substitute effectively for T cell helper activity (Ridge et al. (1998) Nature 393: 474-478) and can be used in conjunction with the CD73 antagonist antibodies described herein, e.g., in combination with a PD-1/PD-L1 axis antagonist antibody. Activating antibodies to T cell costimulatory molecules such as OX-40 (Weinberg et al. (2000) Immunol 164: 2160-2169), 4-1BB (Melero et al. (1997) Nature Medicine 3: 682-685 (1997), and ICOS (Hutloff et al. (1999) Nature 397: 262-266) may also provide for increased levels of T cell activation. Inhibitors of CTLA-4 (e.g., U.S. Pat. No. 5,811,097), may also be used in conjunction with a CD73 antagonist antibodies described herein, e.g., in combination with a PD-1/PD-L1 axis antagonist antibody.

Other methods described herein are used to treat patients that have been exposed to particular toxins or pathogens. Accordingly, provided herein is a method of treating an infectious disease in a subject comprising administering to the subject a CD73 antagonist antibodies described herein, e.g., in combination with a PD-1/PD-L1 axis antagonist antibody, such that the subject is treated for the infectious disease. Additionally or alternatively, the antibody can be a chimeric or humanized antibody.

In all of the above methods, the CD73 antagonist antibodies described herein, e.g., in combination with a PD-1/PD-L1 axis antagonist antibody, can be combined with other forms of immunotherapy such as cytokine treatment (e.g., interferons, GM-CSF, G-CSF, IL-2), or bispecific antibody therapy, which provides for enhanced presentation of tumor antigens (see, e.g., Holliger (1993) Proc. Nat. Acad. Sci. USA 90:6444-6448; Poljak (1994) Structure 2:1121-1123).

In addition to the combinations therapies described above, the CD73 antagonist antibodies described herein, e.g., in combination with a PD-1/PD-L1 axis antagonist antibody, can also be used in combination therapy, e.g., for treating cancer, as described below.

Further provided herein are methods of combination therapy in which CD73 antagonist antibodies described herein, e.g., in combination with a PD-1/PD-L1 axis antagonist antibody, is coadministered with one or more additional agents that are effective in stimulating immune responses to thereby further enhance, stimulate or upregulate immune responses in a subject.

Generally, the CD73 antagonist antibodies described herein, e.g., in combination with a PD-1/PD-L1 axis antagonist antibody, can be further combined with (i) an agonist of a co-stimulatory receptor and/or (ii) an antagonist of an inhibitory signal on T cells, both of which result in amplifying antigen-specific T cell responses (immune checkpoint regulators). Most of the co-stimulatory and co-inhibitory molecules are members of the immunoglobulin super family (IgSF), and CD73 antagonist antibodies described herein may be administered with an agent that targets a member of the IgSF family to increase an immune response. One important family of membrane-bound ligands that bind to co-stimulatory or co-inhibitory receptors is the B7 family, which includes B7-1, B7-2, B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which include CD40 and CD40L, OX-40, OX-40L, CD70, CD27L, CD30, CD30L, 4-1BBL, CD137, GITR, TRAIL/Apo2-L, TRAILR1/DR4, TRAILR2/DR5, TRAILR3, TRAILR4, OPG, RANK, RANKL, TWEAKR/Fnl4, TWEAK, BAFFR, EDAR, XEDAR, TACI, APRIL, BCMA, LTβR, LIGHT, DcR3, HVEM, VEGI/TL1A, TRAMP/DR3, EDAR, EDA1, XEDAR, EDA2, TNFR1, Lymphotoxin α/TNFβ, TNFR2, TNFα, LTβR, Lymphotoxin α1β2, FAS, FASL, RELT, DR6, TROY, NGFR (see, e.g., Tansey (2009) Drug Discovery Today 00:1). T cell activation is also regulated by soluble cytokines. Thus, the CD73 antagonist antibodies described herein, e.g., in combination with a PD-1/PD-L1 axis antagonist antibody, can be used in combination with (i) antagonists (or inhibitors or blocking agents) of proteins of the IgSF family or B7 family or the TNF family that inhibit T cell activation or antagonists of cytokines that inhibit T cell activation (e.g., IL-6, IL-10, TGF-β, VEGF; “immunosuppressive cytokines”) and/or (ii) agonists of stimulatory receptors of the IgSF family, B7 family or the TNF family or of cytokines that stimulate T cell activation, for stimulating an immune response, e.g., for treating proliferative diseases, such as cancer.

For example, T cell responses can be stimulated by the CD73 antagonist antibodies described herein (e.g., CD73.A), e.g., in combination with a PD-1/PD-L1 axis antagonist antibody, and one or more of the following agents:
(1) An antagonist (inhibitor or blocking agent) of a protein that inhibits T cell activation (e.g., immune checkpoint inhibitors), such as CTLA-4, PD-L2, and LAG-3, as described above, and any of the following proteins: TIM-3, Galectin 9, CEACAM-1, BTLA, CD69, Galectin-1, TIGIT, CD113, GPR56, VISTA, 2B4, CD48, GARP, CD73, PD1H, LAIR1, TIM-1, TIM-4, CD39.
(2) An agonist of a protein that stimulates T cell activation, such as B7-1, B7-2, CD28, 4-1BB (CD137), 4-1BBL, GITR, GITRL, ICOS, ICOS-L, OX40, OX40L, CD70, CD27, CD40, DR3 and CD28H.

Exemplary agents that modulate one of the above proteins and may be combined with the CD73 antagonist antibodies described herein, e.g., in combination with a PD-1/PD-L1 axis antagonist antibody (e.g., nivolumab), for treating cancer, include: Yervoy™ (ipilimumab) or Tremelimumab (to CTLA-4), galiximab (to B7.1), BMS-936558 (to PD-1), CT-011 (to PD-1), MK-3475 (to PD-1), AMP224 (to B7DC), BMS-936559 (to B7-H1), MPDL3280A (to B7-H1), MEDI-570 (to ICOS), AMG557 (to B7H2), MGA271 (to B7H3), IMP321 (to LAG-3), BMS-663513 (to CD137), PF-05082566 (to CD137), CDX-1127 (to CD27), anti-OX40 (Providence Health Services), huMAbOX40L (to OX40L), Atacicept (to TACI), CP-870893 (to CD40), Lucatumumab (to CD40), Dacetuzumab (to CD40), Muromonab-CD3 (to CD3), Ipilumumab (to CTLA-4).

Other molecules that can be combined with the CD73 antagonist antibodies described herein, e.g., in combination with a PD-1/PD-L1 axis antagonist antibody, for the treatment of cancer include antagonists of inhibitory receptors on NK cells or agonists of activating receptors on NK cells (e.g., antagonists of KIR (e.g., lirilumab)).

T cell activation is also regulated by soluble cytokines, and the CD73 antagonist antibodies described herein, e.g., in combination with a PD-1/PD-L1 axis antagonist antibody, may be administered to a subject, e.g., having cancer, with antagonists of cytokines that inhibit T cell activation or agonists of cytokines that stimulate T cell activation.

In certain embodiments, the CD73 antagonist antibodies described herein, e.g., in combination with a PD-1/PD-L1 axis antagonist antibody, can be used in combination with (i) antagonists (or inhibitors or blocking agents) of proteins of the IgSF family or B7 family or the TNF family that inhibit T cell activation or antagonists of cytokines that inhibit T cell activation (e.g., IL-6, IL-10, TGF-β, VEGF; “immunosuppressive cytokines”) and/or (ii) agonists of stimulatory receptors of the IgSF family, B7 family or the TNF family or of cytokines that stimulate T cell activation, for stimulating an immune response, e.g., for treating proliferative diseases, such as cancer.

Yet other agents for combination therapies include agents that inhibit or deplete macrophages or monocytes, including but not limited to CSF-1R antagonists such as CSF-1R antagonist antibodies including RG7155 (WO11/70024, WO11/107553, WO11/131407, WO13/87699, WO13/119716, WO13/132044) or FPA-008 (WO11/140249; WO13169264; WO14/036357).

The CD73 antagonist antibodies described herein, e.g., in combination with a PD-1/PD-L1 axis antagonist antibody, may also be administered with agents that inhibit TGF-β signaling.

Additional agents that may be combined with the CD73 antagonist antibodies described herein, e.g., in combination with a PD-1/PD-L1 axis antagonist antibody, include agents that enhance tumor antigen presentation, e.g., dendritic cell vaccines, GM-CSF secreting cellular vaccines, CpG oligonucleotides, and imiquimod, or therapies that enhance the immunogenicity of tumor cells (e.g., anthracyclines).

Yet other therapies that may be combined with the CD73 antagonist antibodies described herein, e.g., in combination with a PD-1/PD-L1 axis antagonist antibody, include therapies that deplete or block Treg cells, e.g., an agent that specifically binds to CD25.

Another therapy that may be combined with the CD73 antagonist antibodies described herein, e.g., in combination with a PD-1/PD-L1 axis antagonist antibody, is a therapy that inhibits a metabolic enzyme such as indoleamine dioxigenase (IDO), dioxigenase, arginase, or nitric oxide synthetase.

Another class of agents that may be used with the CD73 antagonist antibodies described herein, e.g., in combination with a PD-1/PD-L1 axis antagonist antibody, includes agents that inhibit the formation of adenosine or inhibit the adenosine A2A receptor.

Other therapies that may be combined with the CD73 antagonist antibodies described herein, e.g., in combination with a PD-1/PD-L1 axis antagonist antibody, for treating cancer include therapies that reverse/prevent T cell anergy or exhaustion and therapies that trigger an innate immune activation and/or inflammation at a tumor site.

The CD73 antagonist antibodies described herein, e.g., in combination with a PD-1/PD-L1 axis antagonist antibody, may be combined with more than one immuno-oncology agent, and may be, e.g., combined with a combinatorial approach that targets multiple elements of the immune pathway, such as one or more of the following: a therapy that enhances tumor antigen presentation (e.g., dendritic cell vaccine, GM-CSF secreting cellular vaccines, CpG oligonucleotides, imiquimod); a therapy that inhibits negative immune regulation e.g., by inhibiting CTLA-4 and/or depleting or blocking Tregs or other immune suppressing cells; a therapy that stimulates positive immune regulation, e.g., with agonists that stimulate the CD-137, OX-40, and/or GITR pathway and/or stimulate T cell effector function; a therapy that increases systemically the frequency of anti-tumor T cells; a therapy that depletes or inhibits Tregs, such as Tregs in the tumor, e.g., using an antagonist of CD25 (e.g., daclizumab) or by ex vivo anti-CD25 bead depletion; a therapy that impacts the function of suppressor myeloid cells in the tumor; a therapy that enhances immunogenicity of tumor cells (e.g., anthracyclines); adoptive T cell or NK cell transfer including genetically modified cells, e.g., cells modified by chimeric antigen receptors (CAR-T therapy); a therapy that inhibits a metabolic enzyme such as indoleamine dioxigenase (IDO), dioxigenase, arginase, or nitric oxide synthetase; a therapy that reverses/prevents T cell anergy or exhaustion; a therapy that triggers an innate immune activation and/or inflammation at a tumor site; administration of immune stimulatory cytokines; or blocking of immunorepressive cytokines.

Generally, the CD73 antagonist antibodies described herein, e.g., in combination with a PD-1/PD-L1 axis antagonist antibody, can be used together with one or more of agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment, deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell anergy or exhaustion) and agents that trigger innate immune activation and/or inflammation at tumor sites. An increased internalization of inhibitory receptors may translate into a lower level of a potential inhibitor (assuming that signaling does not ensue).

In certain embodiments, the CD73 antagonist antibodies described herein, e.g., in combination with a PD-1/PD-L1 axis antagonist antibody, is administered to a subject together with a BRAF inhibitor if the subject is BRAF V600 mutation positive.

Provided herein are methods for treating a hyperproliferative disease (e.g., cancer), comprising administering the CD73 antagonist antibodies described herein, e.g., in combination with a PD-1/PD-L1 axis antagonist antibody, to a subject. In certain embodiments, the CD73 antagonist antibody is administered at a subtherapeutic dose, the PD-1/PD-L1 axis antagonist antibody is administered at a subtherapeutic dose, or both are administered at a subtherapeutic dose. Also provided herein are methods for altering an adverse event associated with treatment of a hyperproliferative disease with an immunostimulatory agent, comprising administering a CD73 antagonist antibody and a subtherapeutic dose of a PD-1/PD-L1 axis antagonist antibody to a subject. In certain embodiments, the PD-1/PD-L1 axis antagonist antibody is a human sequence monoclonal antibody and the CD73 antagonist antibody is human sequence monoclonal antibody, such as an antibody comprising the CDRs or variable regions of 11F11, 4C3, 4D4, 10D2, 11A6, 24H2, 5F8, 6E11, 7A11, CD73.3, CD73.4, CD73.5, CD73.6, CD73.7, CD73.8, CD73.9, CD73.10 or CD73.11, as described in WO2016/081748 and WO2017/152085, or another CD73 antagonist antibody described herein.

Administration of the CD73 antagonist antibodies described herein, e.g., in combination with a PD-1/PD-L1 axis antagonist antibody, and antagonists, e.g., antagonist antibodies, to one or more second target antigens such as LAG-3 and/or CTLA-4, can enhance the immune response to cancerous cells in the patient. Cancers whose growth may be inhibited using the antibodies of the instant disclosure include cancers typically responsive to immunotherapy.

In certain embodiments, the combination of therapeutic antibodies discussed herein can be administered concurrently as a single composition in a pharmaceutically acceptable carrier, or concurrently as separate compositions with each antibody in a pharmaceutically acceptable carrier. In one embodiment, the combination of therapeutic antibodies can be administered sequentially. Furthermore, if more than one dose of the combination therapy is administered sequentially, the order of the sequential administration can be reversed or kept in the same order at each time point of administration, sequential administrations can be combined with concurrent administrations, or any combination thereof.

In one embodiment, a subject having a disease that may benefit from stimulation of the immune system, e.g., cancer or an infectious disease, is treated by administration to the subject the CD73 antagonist antibodies described herein, e.g., in combination with a PD-1/PD-L1 axis antagonist antibody (e.g., nivolumab), and an immuno-oncology agent, wherein the immuno-oncology agent is a CD137 (4-1BB) agonist, such as an agonistic CD137 antibody. Suitable CD137 antibodies include, for example, urelumab or PF-05082566 (WO12/32433).

In one embodiment, a subject having a disease that may benefit from stimulation of the immune system, e.g., cancer or an infectious disease, is treated by administration to the subject the CD73 antagonist antibodies described herein, e.g., in combination with a PD-1/PD-L1 axis antagonist antibody (e.g., nivolumab), and an immuno-oncology agent, wherein the immuno-oncology agent is an OX40 agonist, such as an agonistic OX40 antibody. Suitable OX40 antibodies include, for example, MEDI-6383, MEDI-6469 or MOXR0916 (RG7888; WO06/029879).

In one embodiment, a subject having a disease that may benefit from stimulation of the immune system, e.g., cancer or an infectious disease, is treated by administration to the subject the CD73 antagonist antibodies described herein, e.g., in combination with a PD-1/PD-L1 axis antagonist antibody (e.g., nivolumab), and an immuno-oncology agent, wherein the immuno-oncology agent is a CD40 agonist, such as an agonistic CD40 antibody. In certain embodiments, the immuno-oncology agent is a CD40 antagonist, such as an antagonistic CD40 antibody. Suitable CD40 antibodies include, for example, lucatumumab (HCD122), dacetuzumab (SGN-40), CP-870,893 or Chi Lob 7/4.

In one embodiment, a subject having a disease that may benefit from stimulation of the immune system, e.g., cancer or an infectious disease, is treated by administration to the subject the CD73 antagonist antibodies described herein, e.g., in combination with a PD-1/PD-L1 axis antagonist antibody (e.g., nivolumab), and an immuno-oncology agent, wherein the immuno-oncology agent is a CD27 agonist, such as an agonistic CD27 antibody. Suitable CD27 antibodies include, for example, varlilumab (CDX-1127).

In one embodiment, a subject having a disease that may benefit from stimulation of the immune system, e.g., cancer or an infectious disease, is treated by administration to the subject the CD73 antagonist antibodies described herein, e.g., in combination with a PD-1/PD-L1 axis antagonist antibody (e.g., nivolumab), and an immuno-oncology agent, wherein the immuno-oncology agent is MGA271 (to B7H3) (WO11/109400).

In one embodiment, a subject having a disease that may benefit from stimulation of the immune system, e.g., cancer or an infectious disease, is treated by administration to the subject the CD73 antagonist antibodies described herein, e.g., in combination with a PD-1/PD-L1 axis antagonist antibody (e.g., nivolumab), and an immuno-oncology agent, wherein the immuno-oncology agent is a KIR antagonist, such as lirilumab.

In one embodiment, a subject having a disease that may benefit from stimulation of the immune system, e.g., cancer or an infectious disease, is treated by administration to the subject the CD73 antagonist antibodies described herein, e.g., in combination with a PD-1/PD-L1 axis antagonist antibody (e.g., nivolumab), and an immuno-oncology agent, wherein the immuno-oncology agent is an IDO antagonist. Suitable IDO antagonists include, for example, INCB-024360 (WO2006/122150, WO07/75598, WO08/36653, WO08/36642), indoximod, NLG-919 (WO09/73620, WO09/1156652, WO11/56652, WO12/142237) or F001287.

In one embodiment, a subject having a disease that may benefit from stimulation of the immune system, e.g., cancer or an infectious disease, is treated by administration to the subject the CD73 antagonist antibodies described herein, e.g., in combination with a PD-1/PD-L1 axis antagonist antibody (e.g., nivolumab), and an immuno-oncology agent, wherein the immuno-oncology agent is a Toll-like receptor agonist, e.g., a TLR2/4 agonist (e.g., Bacillus Calmette-Guerin); a TLR7 agonist (e.g., Hiltonol or Imiquimod); a TLR7/8 agonist (e.g., Resiquimod); or a TLR9 agonist (e.g., CpG7909).

In one embodiment, a subject having a disease that may benefit from stimulation of the immune system, e.g., cancer or an infectious disease, is treated by administration to the subject the CD73 antagonist antibodies described herein, e.g., in combination with a PD-1/PD-L1 axis antagonist antibody (e.g., nivolumab), and an immuno-oncology agent, wherein the immuno-oncology agent is a TGF-β inhibitor, e.g., GC1008, LY2157299, TEW7197, or IMC-TR1.

In certain embodiments, the CD73 antagonist antibody is sequentially administered prior to administration of a second agent, e.g., the PD-1/PD-L1 axis antagonist antibody and/or immuno-oncology agent described above. In certain embodiments, the CD73 antagonist antibody is administered concurrently with the second agent, e.g., the PD-1/PD-L1 axis antagonist antibody and/or immunology-oncology agent described above. In certain embodiments, the CD73 antagonist antibody is sequentially administered after administration of the second agent, e.g., the PD-1/PD-L1 axis antagonist antibody and/or immunology-oncology agent described above. The administration of the two or more agents may start at times that are, e.g., 30 minutes, 60 minutes, 90 minutes, 120 minutes, 3 hours, 6 hours, 12 hours, 24 hours, 36 hours, 48 hours, 3 days, 5 days, 7 days, or one or more weeks apart, or administration of the second and/or further agent may start, e.g., 30 minutes, 60 minutes, 90 minutes, 120 minutes, 3 hours, 6 hours, 12 hours, 24 hours, 36 hours, 48 hours, 3 days, 5 days, 7 days, or one or more weeks after the first agent has been administered. In certain embodiments, the CD73 antagonist antibody and a second and/or further agent are infused simultaneously, e.g., over a period of 30 or 60 minutes, to a patient. A CD73 antagonist antibody may be co-formulated with a second and/or further agent.

Optionally, the CD73 antagonist antibodies described herein, e.g., in combination with a PD-1/PD-L1 axis antagonist antibody (e.g., nivolumab), optionally in combination with one or more additional immunotherapeutic antibodies (e.g., anti-CTLA-4 and/or anti-LAG-3 blockade) can be further combined with an immunogenic agent, such as cancerous cells, purified tumor antigens (including recombinant proteins, peptides, and carbohydrate molecules), cells, and cells transfected with genes encoding immune stimulating cytokines (He et al. (2004) J. Immunol. 173:4919-28). Non-limiting examples of tumor vaccines that can be used include peptides of melanoma antigens, such as peptides of gp100, MAGE antigens, Trp-2, MART1 and/or tyrosinase, or tumor cells transfected to express the cytokine GM-CSF (discussed further below). The CD73 antagonist antibodies described herein, e.g., in combination with a PD-1/PD-L1 axis antagonist antibody (e.g., nivolumab), can also be further combined with standard cancer treatments. For example, the CD73 antagonist antibodies described herein, e.g., in combination with a PD-1/PD-L1 axis antagonist antibody (e.g., nivolumab), and one or more additional antibodies (e.g., CTLA-4 and/or LAG-3 blockade) can be effectively combined with chemotherapeutic regimes. In these instances, it is possible to reduce the dose of other chemotherapeutic reagent administered with the combination of the instant disclosure (Mokyr et al. (1998) Cancer Research 58: 5301-5304). Exemplary chemotherapeutic regimens include decarbazine for the treatment of melanoma and interleukin-2 (IL-2) for the treatment of melanoma.

Other combination therapies that may result in synergy with the CD73 antagonist antibodies described herein, e.g., in combination with a PD-1/PD-L1 axis antagonist antibody, with or without and CTLA-4 and/or LAG-3 blockade, through cell death include radiation, surgery, or hormone deprivation. Each of these protocols creates a source of tumor antigen in the host. Angiogenesis inhibitors can also be combined with the CD73 antagonist antibodies described herein, e.g., in combination with a PD-1/PD-L1 axis antagonist antibody, and CTLA-4 and/or LAG-3 blockade. Inhibition of angiogenesis leads to tumor cell death, which can be a source of tumor antigen fed into host antigen presentation pathways.

The CD73 antagonist antibodies described herein, e.g., in combination with a PD-1/PD-L1 axis antagonist antibody, can also be used in combination with bispecific antibodies that target Fcα or Fcγ receptor-expressing effector cells to tumor cells (see, e.g., U.S. Pat. Nos. 5,922,845 and 5,837,243). Bispecific antibodies can be used to target two separate antigens. The T cell arm of these responses would be augmented by the use of a combined CD73 inhibition and PD-1 and/or PD-L1 blockade.

In another example, the CD73 antagonist antibodies described herein, e.g., in combination with a PD-1/PD-L1 axis antagonist antibody, can be used in conjunction with an anti-neoplastic antibody, such as Rituxan® (rituximab), Herceptin® (trastuzumab), Bexxar® (tositumomab), Zevalin® (ibritumomab), Campath® (alemtuzumab), Lymphocide® (eprtuzumab), Avastin® (bevacizumab), and Tarceva® (erlotinib), and the like. By way of example and not wishing to be bound by theory, treatment with an anti-cancer antibody or an anti-cancer antibody conjugated to a toxin can lead to cancer cell death (e.g., tumor cells) which would potentiate an immune response mediated by the immunostimulating agent, e.g., CD73, CTLA-4, PD-1, PD-L1 or LAG-3 agent, e.g., antibody.

Tumors evade host immune surveillance by a large variety of mechanisms. Many of these mechanisms may be overcome by the inactivation of proteins, which are expressed by the tumors and which are immunosuppressive. These include, among others, TGF-β (Kehrl et al. (1986) J. Exp. Med. 163: 1037-1050), IL-10 (Howard & O'Garra (1992) Immunology Today 13: 198-200), and Fas ligand (Hahne et al. (1996) Science 274: 1363-1365). Antibodies to each of these entities can be further combined with the CD73 antagonist antibodies described herein, e.g., in combination with a PD-1/PD-L1 axis antagonist antibody, to counteract the effects of immunosuppressive agents and favor anti-tumor immune responses by the host.

Other agents, e.g., antibodies, that can be used to activate host immune responsiveness can be further used in combination with the CD73 antagonist antibodies described herein, e.g., in combination with a PD-1/PD-L1 axis antagonist antibody.

These include molecules on the surface of dendritic cells that activate DC function and antigen presentation. Anti-CD40 antibodies (Ridge et al., supra) can be used in conjunction with the CD73 antagonist antibodies described herein, e.g., in combination with a PD-1/PD-L1 axis antagonist antibody. Other activating antibodies to T cell costimulatory molecules Weinberg et al., supra, Melero et al. supra, Hutloff et al., supra, may also provide for increased levels of T cell activation.

As discussed above, bone marrow transplantation is currently being used to treat a variety of tumors of hematopoietic origin. The CD73 antagonist antibodies described herein, e.g., in combination with a PD-1/PD-L1 axis antagonist antibody, can be used to increase the effectiveness of the donor engrafted tumor specific T cells.

Several experimental treatment protocols involve ex vivo activation and expansion of antigen specific T cells and adoptive transfer of these cells into recipients in order to antigen-specific T cells against tumor (Greenberg & Riddell, supra). These methods can also be used to activate T cell responses to infectious agents such as CMV. Ex vivo activation in the presence of the CD73 antagonist antibodies described herein, e.g., in combination with a PD-1/PD-L1 axis antagonist antibody, can be expected to increase the frequency and activity of the adoptively transferred T cells.

Provided herein are methods for altering an adverse event associated with treatment of a hyperproliferative disease (e.g., cancer) with an immunostimulatory agent, comprising administering the CD73 antagonist antibodies described herein, e.g., in combination with a subtherapeutic dose of PD-1/PD-L1 axis antagonist antibody (e.g., nivolumab), to a subject. For example, the methods described herein provide for a method of reducing the incidence of immunostimulatory therapeutic antibody-induced colitis or diarrhea by administering a non-absorbable steroid to the patient. As used herein, a “non-absorbable steroid” is a glucocorticoid that exhibits extensive first pass metabolism such that, following metabolism in the liver, the bioavailability of the steroid is low, i.e., less than about 20%. In one embodiment described herein, the non-absorbable steroid is budesonide. Budesonide is a locally-acting glucocorticosteroid, which is extensively metabolized, primarily by the liver, following oral administration. ENTOCORT EC® (Astra-Zeneca) is a pH- and time-dependent oral formulation of budesonide developed to optimize drug delivery to the ileum and throughout the colon. ENTOCORT EC® is approved in the U.S. for the treatment of mild to moderate Crohn's disease involving the ileum and/or ascending colon. The usual oral dosage of ENTOCORT EC® for the treatment of Crohn's disease is 6 to 9 mg/day. ENTOCORT EC® is released in the intestines before being absorbed and retained in the gut mucosa. Once it passes through the gut mucosa target tissue, ENTOCORT EC® is extensively metabolized by the cytochrome P450 system in the liver to metabolites with negligible glucocorticoid activity. Therefore, the bioavailability is low (about 10%). The low bioavailability of budesonide results in an improved therapeutic ratio compared to other glucocorticoids with less extensive first-pass metabolism. Budesonide results in fewer adverse effects, including less hypothalamic-pituitary suppression, than systemically-acting corticosteroids. However, chronic administration of ENTOCORT EC® can result in systemic glucocorticoid effects such as hypercorticism and adrenal suppression. See PDR 58^{th} ed. 2004; 608-610.

In still further embodiments, the CD73 antagonist antibodies described herein, e.g., in combination with a PD-1/PD-L1 axis antagonist antibody, in conjunction with a non-absorbable steroid can be further combined with a salicylate. Salicylates include 5-ASA agents such as, for example: sulfasalazine (AZULFIDINE®, Pharmacia & UpJohn); olsalazine (DIPENTUM®, Pharmacia & UpJohn); balsalazide (COLAZAL®, Salix Pharmaceuticals, Inc.); and mesalamine (ASACOL®, Procter & Gamble Pharmaceuticals; PENTASA®, Shire US; CANASA®, Axcan Scandipharm, Inc.; ROWASA®, Solvay).

In accordance with the methods described herein, a salicylate administered in combination with the CD73 antagonist antibodies described herein, e.g., in combination with a PD-1/PD-L1 axis antagonist antibody, and a non-absorbable steroid can includes any overlapping or sequential administration of the salicylate and the non-absorbable steroid for the purpose of decreasing the incidence of colitis induced by the immunostimulatory antibodies. Thus, for example, methods for reducing the incidence of colitis induced by the immunostimulatory antibodies described herein encompass administering a salicylate and a non-absorbable concurrently or sequentially (e.g., a salicylate is administered 6 hours after a non-absorbable steroid), or any combination thereof. Further, a salicylate and a non-absorbable steroid can be administered by the same route (e.g., both are administered orally) or by different routes (e.g., a salicylate is administered orally and a non-absorbable steroid is administered rectally), which may differ from the route(s) used to administer the CD73 antagonist antibodies or PD-1/PD-L1 axis antagonist antibodies.

The CD73 antagonist antibodies described herein, e.g., in combination with a PD-1/PD-L1 axis antagonist antibody, and further combination antibody therapies described herein may also be used in conjunction with other well known therapies that are selected for their particular usefulness against the indication being treated (e.g., cancer).

For example, the CD73 antagonist antibodies described herein, e.g., in combination with a PD-1/PD-L1 axis antagonist antibody, can be used in combination (e.g., simultaneously or separately) with an additional treatment, such as irradiation, chemotherapy (e.g., using camptothecin (CPT-11), 5-fluorouracil (5-FU), cisplatin, doxorubicin, irinotecan, paclitaxel, gemcitabine, cisplatin, paclitaxel, carboplatin-paclitaxel (Taxol), doxorubicin, 5-fu, or camptothecin+apo2l/TRAIL (a 6× combo)), one or more proteasome inhibitors (e.g., bortezomib or MG132), one or more Bcl-2 inhibitors (e.g., BH3I-2′ (bcl-xl inhibitor), indoleamine dioxygenase-1 (IDO1) inhibitor (e.g., INCB24360), AT-101 (R-(−)-gossypol derivative), ABT-263 (small molecule), GX-15-070 (obatoclax), or MCL-1 (myeloid leukemia cell differentiation protein-1) antagonists), iAP (inhibitor of apoptosis protein) antagonists (e.g., smac7, smac4, small molecule smac mimetic, synthetic smac peptides (see Fulda et al., Nat Med 2002; 8:808-15), ISIS23722 (LY2181308), or AEG-35156 (GEM-640)), HDAC (histone deacetylase) inhibitors, anti-CD20 antibodies (e.g., rituximab), angiogenesis inhibitors (e.g., bevacizumab), anti-angiogenic agents targeting VEGF and VEGFR (e.g., Avastin), synthetic triterpenoids (see Hyer et al., Cancer Research 2005; 65:4799-808), c-FLIP (cellular FLICE-inhibitory protein) modulators (e.g., natural and synthetic ligands of PPARγ (peroxisome proliferator-activated receptor γ), 5809354 or 5569100), kinase inhibitors (e.g., Sorafenib), Trastuzumab, Cetuximab, Temsirolimus, mTOR inhibitors such as rapamycin and temsirolimus, Bortezomib, JAK2 inhibitors, HSP90 inhibitors, PI3K-AKT inhibitors, Lenalildomide, GSK30 inhibitors, IAP inhibitors and/or genotoxic drugs.

The CD73 antagonist antibodies described herein, e.g., in combination with a PD-1/PD-L1 axis antagonist antibody, can further be used in combination with one or more anti-proliferative cytotoxic agents. Classes of compounds that may be used as anti-proliferative cytotoxic agents include, but are not limited to, the following:

Alkylating agents (including, without limitation, nitrogen mustards, ethylenimine derivatives, alkyl sulfonates, nitrosoureas and triazenes): Uracil mustard, Chlormethine, Cyclophosphamide (CYTOXAN™) fosfamide, Melphalan, Chlorambucil, Pipobroman, Triethylenemelamine, Triethylenethiophosphoramine, Busulfan, Carmustine, Lomustine, Streptozocin, Dacarbazine, and Temozolomide.

Antimetabolites (including, without limitation, folic acid antagonists, pyrimidine analogs, purine analogs and adenosine deaminase inhibitors): Methotrexate, 5-Fluorouracil, Floxuridine, Cytarabine, 6-Mercaptopurine, 6-Thioguanine, Fludarabine phosphate, Pentostatine, and Gemcitabine.

Suitable anti-proliferative agents for combining with the CD73 antagonist antibodies described herein, e.g., in combination with a PD-1/PD-L1 axis antagonist antibody (e.g., nivolumab), include, without limitation, taxanes, paclitaxel (paclitaxel is commercially available as TAXOL™), docetaxel, discodermolide (DDM), dictyostatin (DCT), Peloruside A, epothilones, epothilone A, epothilone B, epothilone C, epothilone D, epothilone E, epothilone F, furanoepothilone D, desoxyepothilone B1, [17]-dehydrodesoxyepothilone B, [18]dehydrodesoxyepothilones B, C12,13-cyclopropyl-epothilone A, C6-C8 bridged epothilone A, trans-9,10-dehydroepothilone D, cis-9,10-dehydroepothilone D, 16-desmethylepothilone B, epothilone B10, discoderomolide, patupilone (EPO-906), KOS-862, KOS-1584, ZK-EPO, ABJ-789, XAA296A (Discodermolide), TZT-1027 (soblidotin), ILX-651 (tasidotin hydrochloride), Halichondrin B, Eribulin mesylate (E-7389), Hemiasterlin (HTI-286), E-7974, Cyrptophycins, LY-355703, Maytansinoid immunoconjugates (DM-1), MKC-1, ABT-751, T1-38067, T-900607, SB-715992 (ispinesib), SB-743921, MK-0731, STA-5312, eleutherobin, 17beta-acetoxy-2-ethoxy-6-oxo-B-homo-estra-1,3,5(10)-trien-3-ol, cyclostreptin, isolaulimalide, laulimalide, 4-epi-7-dehydroxy-14,16-didemethyl-(+)-discodermolides, and cryptothilone 1, in addition to other microtubuline stabilizing agents known in the art.

In cases where it is desirable to render aberrantly proliferative cells quiescent in conjunction with or prior to treatment with the CD73 antagonist antibodies described herein, e.g., in combination with a PD-1/PD-L1 axis antagonist antibody, hormones and steroids (including synthetic analogs), such as 17a-Ethinylestradiol, Diethylstilbestrol, Testosterone, Prednisone, Fluoxymesterone, Dromostanolone propionate, Testolactone, Megestrolacetate, Methylprednisolone, Methyl-testosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, ZOLADEX™, can also be administered to the patient. When employing the methods or compositions described herein, other agents used in the modulation of tumor growth or metastasis in a clinical setting, such as antimimetics, can also be administered as desired.

Methods for the safe and effective administration of chemotherapeutic agents are known to those skilled in the art. In addition, their administration is described in the standard literature. For example, the administration of many of the chemotherapeutic agents is described in the Physicians' Desk Reference (PDR), e.g., 1996 edition (Medical Economics Company, Montvale, N.J. 07645-1742, USA); the disclosure of which is incorporated herein by reference thereto.

The chemotherapeutic agent(s) and/or radiation therapy can be administered according to therapeutic protocols well known in the art. It will be apparent to those skilled in the art that the administration of the chemotherapeutic agent(s) and/or radiation therapy can be varied depending on the disease being treated and the known effects of the chemotherapeutic agent(s) and/or radiation therapy on that disease. Also, in accordance with the knowledge of the skilled clinician, the therapeutic protocols (e.g., dosage amounts and times of administration) can be varied in view of the observed effects of the administered therapeutic agents on the patient, and in view of the observed responses of the disease to the administered therapeutic agents.

### II. CD73 Antagonist Antibodies

The CD73 antagonist antibodies that are suitable for use in the methods described herein include newly developed CD73 antagonist antibodies, as well as CD73 antagonist antibodies known in the art (including antibodies that compete with or bind to the same epitope as the antibodies).

Exemplary CD73 antagonist antibodies for use in the methods described herein are MEDI9447 and CPX-006, and antibodies described in WO2016/081748 and WO2017/152085, the contents of which are herein incorporated by reference in their entireties.

In certain embodiments, the CD73 antagonistic antibody exhibits 1, 2, 3, 4, 5, 6, 7, 8, 9, 10, or 11 of the following properties:
(1) binding to human CD73, e.g., bead bound human dimeric human CD73 isoform 1 and 2, e.g., with a K_{D} of 10 nM or less (e.g., 0.01 nM to 10 nM), e.g., as measured by BIACORE® SPR analysis;
(2) binding to membrane bound human CD73, e.g., with an EC₅₀ of 1 nM or less (e.g., 0.01 nM to 1 nM);
(3) binding to cynomolgus CD73, e.g., binding to membrane bound cynomolgus CD73, e.g, with an EC₅₀ of 10 nM or less (e.g., 0.01 nM to 10 nM);
(4) inhibition of human CD73 enzymatic activity, e.g., with an EC50 of 10 nM or less;
(5) inhibition of cyno CD73 enzymatic activity, e.g., with an EC50 of 10 nM or less;
(6) inhibition of endogenous (cellular) human CD73 enzymatic activity in Calu6 cells with an EC50 of 10 nM or less;
(7) inhibition of human CD73 enzymatic activity in vivo;
(8) internalization, e.g., antibody mediated (or dependent) CD73 internalization, into cells, e.g., with a T_{1/2} of less than 1 hour, 30 minutes or 10 minutes and/or a Ymax of at least 70%, 80% or 90%;
(9) binding to a conformational epitope on human CD73, e.g., a discontinuous epitope within the amino acid sequence (SEQ ID NO: 1) which includes all or a portion of amino acid residues FTKVQQIRRAEPNVLLLDA (SEQ ID NO: 26) and/or LYLPYKVLPVGDEVVG (SEQ ID NO: 27);
(10) competing in either direction or both directions for binding to human CD73 with CD73.4-1, CD73.4-2, CD73.3, 11F11-1, 11F11-2, 4C3-1, 4C3-2, 4C3-3, 4D4, 10D2-1, 10D2-2, 11A6, 24H2, 5F8-1, 5F8-2, 6E11 and/or 7A11, which are described in WO2016/081748 and WO2017/152085; and
(11) interacting with human CD73 in a similar pattern as CD73.4, as determined by X-ray crystallography.

In a particular embodiment, the CD73 antagonistic antibody used in the methods described herein is CD73.4-IgG2C219S.IgG1.1f, which is disclosed in WO2016/081748 and WO2017/152085 (also referred to herein as “CD73.A”). The heavy and light chain sequences, variable region sequences, and CDR sequences of CD73.A are provided in Tables 1 and 6.

TABLE 1
Summary of CD73.A sequences

| SEQ ID | | CD73.A sequences |
|---|---|---|
| 3 | Heavy chain | QVQLVESGGGVVQPGRSLRLSCAASGFTFSNYGMHWVRQAPGKGLEWV |
| | | AVILYDGSNKYYPDSVKGRFTISRDNSKNTLYLQMNSLRAEDTAVYYC |
| | | ARGGSSWYPDSFDIWGQGTMVTVSSASTKGPSVFPLAPCSRSTSESTA |
| | | ALGCLVKDYFPEPVTVSWNSGALTSGVHTFPAVLQSSGLYSLSSVVTV |
| | | PSSNEGTQTYTCNVDHKPSNTKVDKTVERKSCVECPPCPAPPVAGPSV |
| | | FLFPPKPKDTLMISRTPEVTCVVVDVSHEDPEVKFNWYVDGVEVHNAK |
| | | TKPREEQYNSTYRVVSVLTVLHQDWLNGKEYKCKVSNKALPSSIEKTI |
| | | SKAKGQPREPQVYTLPPSREEMTKNQVSLTCLVKGFYPSDIAVEWESN |
| | | GQPENNYKTTPPVLDSDGSFFLYSKLTVDKSRWQQGNVFSCSVMHEAL |
| | | HNHYTQKSLSLSPGK |
| 4 | Heavy chain | QVQLVESGGGVVQPGRSLRLSCAASGFTFSNYGMHWVRQAPGKGLEWV |
| | w/o C-terminal | AVILYDGSNKYYPDSVKGRFTISRDNSKNTLYLQMNSLRAEDTAVYYC |
| | lysine | ARGGSSWYPDSFDIWGQGTMVTVSSASTKGPSVFPLAPCSRSTSESTA |
| | | ALGCLVKDYFPEPVTVSWNSGALTSGVHTFPAVLQSSGLYSLSSVVTV |
| | | PSSNFGTQTYTCNVDHKPSNTKVDKTVERKSCVECPPCPAPPVAGPSV |
| | | FLFPPKPKDTLMISRTPEVTCVVVDVSHEDPEVKFNWYVDGVEVHNAK |
| | | TKPREEQYNSTYRVVSVLTVLHQDWLNGKEYKCKVSNKALPSSIEKTI |
| | | SKAKGQPREPQVYTLPPSREEMTKNQVSLTCLVKGFYPSDIAVEWESN |
| | | GQPENNYKTTPPVLDSDGSFELYSKLTVDKSRWQQGNVESCSVMHEAL |
| | | HNHYTQKSLSLSPG |
| 5 | Light chain | DIQMTQSPSSLSASVGDRVTITCRASQGISSWLAWYQQKPEKAPKSLI |
| | | YAASSLQSGVPSRFSGSGSGTDFTLTISSLQPEDFATYYCQQYNSYPL |
| | | TFGGGTKVEIKRTVAAPSVFIFPPSDEQLKSGTASVVCLLNNFYPREA |
| | | KVQWKVDNALQSGNSQESVTEQDSKDSTYSLSSTLTLSKADYEKHKVY |
| | | ACEVTHQGLSSPVTKSFNRGEC |
| 6 | VH | QVQLVESGGGVVQPGRSLRLSCAASGFTFSNYGMHWVRQAPGKGLEWV |
| | | AVILYDGSNKYYPDSVKGRFTISRDNSKNTLYLQMNSLRAEDTAVYYC |
| | | ARGGSSWYPDSEDIWGQGTMVIVSS |
| 7 | VL | DIQMTQSPSSLSASVGDRVTITCRASQGISSWLAWYQQKPEKAPKSLI |
| | | YAASSLQSCVPSRFSGSGSGTDFTLTISSLQPEDFATYYCQQYNSYPL |
| | | TFGGGTKVEIK |
| 8 | VHCDR1 | NYGMH |
| 9 | VHCDR2 | VILYDGSNKYYPDSVKG |
| 10 | VHCDR3 | GGSSWYPDSFDI |
| 11 | VLCDR1 | RASQGISSWLA |
| 12 | VLCDR2 | AASSLQS |
| 13 | VLCDR3 | QQYNSYPLT |

Accordingly, in certain embodiments, the CD73 antagonist antibody comprises the three variable heavy chain CDRs and the three variable light chain CDRs that are in the variable heavy chain and variable light chain of SEQ ID NOs: 6 and 7, respectively.

In certain embodiments, the CD73 antagonist antibody comprises heavy chain CDR1, CDR2, and CDR3 sequences comprising SEQ ID NOs: 8, 9, and 10, respectively, and/or light chain CDR1, CDR2, and CDR3 sequences comprising SEQ ID NOs: 11, 12, and 13, respectively.

In certain embodiments, the CD73 antagonist antibody comprises heavy and light chain variable region sequences set forth in SEQ ID NOs: 6 and 7, respectively.

In certain embodiments, the CD73 antagonist antibody comprises a heavy chain sequence set forth in SEQ ID NO: 3 or 4, and a light chain sequence set forth in SEQ ID NO: 5.

Preferably, the CD73 antagonist antibody binds to human CD73 with high affinity, for example, with a K_{D} of 10⁻⁷ M or less, 10⁻⁸ M or less, 10⁻⁹ M or less, 10⁻¹⁰ M or less, 10⁻¹¹ M or less, 10⁻² M or less, 10⁻² M to 10⁻⁷ M, 10⁻¹¹ M to 10⁻⁷ M, 10⁻¹⁰ M to 10⁻⁷ M, or 10⁻⁹ M to 10⁻⁷ M.

In certain embodiments, the CD73 antagonist antibody is selected from the group consisting of an IgG1, an IgG2, an IgG3, an IgG4, or a variant or hybrid thereof.

In certain embodiments, the CD73 antagonist antibody comprises a modified heavy chain constant region that alters the properties of the antibody. For instance, the agonistic antibodies may comprise a modified heavy chain constant region that alters the activity of the antibodies relative to antibodies having a non-modified heavy chain constant region. Accordingly, in some embodiments, the agonistic antibodies have modifications in the heavy chain constant region that enhance effector function. In other embodiments, the agonistic antibodies have modifications in the heavy chain constant region that reduce effector function. Modifications in the Fc region can be made to, for example, (a) increase or decrease antibody-dependent cell-mediated cytotoxicity (ADCC), (b) increase or decrease complement mediated cytotoxicity (CDC), (c) increase or decrease affinity for C1q and/or (d) increase or decrease affinity for a Fc receptor relative to the parent Fc. Specific modifications (e.g., amino acid substitution(s)) that can be made to generate variant Fc regions having these features are well known in the art, and summarized in, e.g., WO2016/081748 and WO2017/152085.

In certain embodiments, the CD73 antagonist antibody comprises a hybrid heavy chain constant region. In certain embodiments, the hybrid heavy chain constant region comprises a human CH1 domain, a human hinge domain, a human CH2 domain, and a human CH3 domain in order from N- to C-terminus, wherein at least 2 of the domains are from different isotypes (i.e., selected from IgG1, IgG2, IgG3, and IgG4). In certain embodiments, the hybrid heavy chain constant region is an IgG2/IgG1 heavy chain constant region. In certain embodiments, the IgG2/IgG1 heavy chain constant region comprises a CH1 and hinge from IgG2 and CH2 and CH3 of IgG1. In certain embodiments, the IgG2/IgG1 heavy chain constant region comprises a CH1 and hinge from IgG2 (with C219S) and CH2 and CH3 of IgG1 (with A330S/P331S). In some embodiments, the IgG2/IgG1 heavy chain constant region comprises or consists of the amino acid sequence set forth in SEQ ID NO: 14.

In some embodiments, the CD73 antagonist antibody is a human or a humanized antibody.

In some embodiments, the CD73 antagonist antibody is a bispecific antibody.

In some embodiments, the CD73 antagonist antibody is an immunoconjugate that is conjugated to a moiety, such as a detectable label (e.g., radioisotopes, fluorescent labels, enzymes, and other suitable antibody tags) or an anti-cancer agent (e.g., antimetabolites, alkylating agents, DNA minor groove binders, DNA intercalators, DNA crosslinkers, histone deacetylase inhibitors, nuclear export inhibitors, proteasome inhibitors, topoisomerase I or II inhibitors, heat shock protein inhibitors, tyrosine kinase inhibitors, antibiotics, and anti-mitotic agents). In some embodiments, the immunoconjugate is an antibody-drug conjugate (ADC).

Also contemplated are CD73 antagonist antibodies which comprise heavy and light chain variable region sequences that are at least 85%, e.g., at least 90, 95, or 98% identical to the heavy and light chain variable region sequences of the antibodies described herein.

### III. PD-1/PD-L1 Axis Antagonist Antibodies

A CD73 antagonist antibody may be administered with a PD-1/PD-L1 axis antagonist antibody in the methods described herein. PD-1 is a key immune checkpoint receptor expressed by activated T and B cells and mediates immunosuppression. PD-1 is a member of the CD28 family of receptors, which includes CD28, CTLA-4, ICOS, PD-1, and BTLA. Two cell surface glycoprotein ligands for PD-1 have been identified, Programmed Death Ligand-1 (PD-L1) and Programmed Death Ligand-2 (PD-L2), that are expressed on antigen-presenting cells as well as many human cancers and have been shown to down regulate T cell activation and cytokine secretion upon binding to PD-1. Inhibition of the PD-1/PD-L1 interaction mediates potent antitumor activity in preclinical models.

HuMAbs that bind specifically to PD-1 with high affinity have been disclosed in U.S. Pat. Nos. 8,008,449 and 8,779,105. Other PD-1/PD-L1 axis antagonist antibodies have been described in, for example, U.S. Pat. Nos. 6,808,710, 7,488,802, 8,168,757 and 8,354,509, and PCT Publication No. WO 2012/145493. Each of the PD-1/PD-L1 axis antagonist antibodies disclosed in U.S. Pat. No. 8,008,449 has been demonstrated to exhibit one or more of the following characteristics: (a) binds to human PD-1 with a K_{D} of 1×10⁻⁷ M or less, as determined by surface plasmon resonance using a Biacore biosensor system; (b) does not substantially bind to human CD28, CTLA-4 or ICOS; (c) increases T-cell proliferation in a Mixed Lymphocyte Reaction (MLR) assay; (d) increases interferon-γ production in an MLR assay; (e) increases IL-2 secretion in an MLR assay; (f) binds to human PD-1 and cynomolgus monkey PD-1; (g) inhibits the binding of PD-L1 and/or PD-L2 to PD-1; (h) stimulates antigen-specific memory responses; (i) stimulates antibody responses; and (j) inhibits tumor cell growth in vivo. PD-1 antagonist antibodies useful for the present invention include antibodies that bind specifically to human PD-1 and exhibit at least one, preferably at least five, of the preceding characteristics.

In one embodiment, the PD-1/PD-L1 axis antagonist antibody is nivolumab. Nivolumab (also known as “OPDIVO®”; formerly designated 5C4, BMS-936558, MDX-1106, or ONO-4538) is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., 2014 Cancer Immunol Res. 2(9):846-56). Nivolumab can also be referred to as BMS-936558, MDX-1106 ONO-4538, or by its CAS Registry No. 946414-94-4, and is disclosed as antibody 5C4 in WO 2006/121168, incorporated herein by reference in its entirety and for all purposes. Nivolumab is a human monoclonal antibody that specifically binds to PD-1 and comprises a heavy chain variable region provided as SEQ ID NO: 18, and a light chain variable region provided as SEQ ID NO: 19. The heavy chain sequence of nivolumab is set forth in SEQ ID NO: 15 and 16, and the light chain sequences of nivolumab is set forth in SEQ ID NO 17. Nivolumab comprises heavy chain CDR1-3 sequences set forth in SEQ ID NOs: 20, 21, and 22, and light chain CDR1-3 sequences set forth in SEQ ID NOs: 23, 24, and 25, respectively. Also contemplated are PD-1/PD-L1 axis antagonist antibodies comprising heavy and light chain variable region sequences that are at least 85%, 90%, 95%, 98%, or 99% identical with the heavy and light chain variable region sequences set forth in SEQ ID NOs: 18 and 19 respectively. In certain embodiments, the PD-1/PD-L1 axis antagonist antibody comprises heavy and light chain sequences that are at least 85%, 90%, 95%, 98%, or 99% identical with the heavy chain sequence set forth in SEQ ID NO: 15 or 16, and the light chain sequence set forth in SEQ ID NO: 17. Pharmaceutical compositions of nivolumab include all pharmaceutically acceptable compositions comprising nivolumab and one or more diluents, vehicles and/or excipients. In certain embodiments, nivolumab is administered intravenously. In certain embodiments, nivolumab is administered subcutaneously.

In one embodiment, the PD-1/PD-L1 axis antagonist antibody is pembrolizumab. Pembrolizumab (also known as “KEYTRUDA®”, lambrolizumab, and MK-3475) is a humanized monoclonal IgG4 antibody directed against human cell surface receptor PD-1 (programmed death-1 or programmed cell death-1). Pembrolizumab is described, for example, in U.S. Pat. Nos. 8,354,509 and 8,900,587; see also http://www.cancer.gov/drugdictionary?cdrid=695789 (last accessed: Dec. 14, 2014). Pembrolizumab has been approved by the FDA for the treatment of relapsed or refractory melanoma.

In one embodiment, the PD-1/PD-L1 axis antagonist antibody is MEDI0608 (formerly AMP-514), which is a monoclonal antibody. MEDI0608 is described, for example, in U.S. Pat. No. 8,609,089B2 or in http://www.cancer.gov/drugdictionary?cdrid=756047 (last accessed Dec. 14, 2014).

In one embodiment, the PD-1/PD-L1 axis antagonist antibody is Pidilizumab (CT-011), which is a humanized monoclonal antibody. Pidilizumab is described in U.S. Pat. No. 8,686,119 B2 or WO 2013/014668 A1. The specificity of CT-011 for PD-1 binding has been questioned.

PD-1/PD-L1 axis antagonist antibodies useful in the methods described herein also include isolated antibodies that bind specifically to human PD-1 and compete or cross-compete for binding to human PD-1 with, or bind to the same epitope on human PD-1 as, nivolumab (see, e.g., U.S. Pat. Nos. 8,008,449 and 8,779,105; WO 2013/173223) or other PD-1 antagonist antibody.

PD-1/PD-L1 axis antagonist antibodies suitable for use in the disclosed compositions are antibodies that bind to PD-1 with high specificity and affinity, block the binding of PD-L1 and or PD-L2, and inhibit the immunosuppressive effect of the PD-1 signaling pathway. In any of the compositions or methods disclosed herein, a PD-1 antagonist antibody includes an antigen-binding portion or fragment that binds to the PD-1 receptor and exhibits the functional properties similar to those of whole antibodies in inhibiting ligand binding and upregulating the immune system. In certain embodiments, the PD-1 antagonist antibody or antigen-binding portion thereof cross-competes with nivolumab for binding to human PD-1.

In certain embodiments, the PD-1/PD-L1 axis antagonist antibodies or antigen-binding portion thereof is a chimeric, humanized or human monoclonal antibodies or a portion thereof. In certain embodiments, the antibody is a humanized antibody. In other embodiments, the antibody is a human antibody. Antibodies of an IgG1, IgG2, IgG3 or IgG4 isotype can be used.

In certain embodiments, the PD-1/PD-L1 axis antagonist antibodies or antigen-binding portion thereof comprises a heavy chain constant region which is of a human IgG1 or IgG4 isotype. In certain other embodiments, the sequence of the IgG4 heavy chain constant region of the PD-1/PD-L1 axis antagonist antibody or antigen-binding portion thereof contains an S228P mutation which replaces a serine residue in the hinge region with the proline residue normally found at the corresponding position in IgG1 isotype antibodies. This mutation, which is present in nivolumab, prevents Fab arm exchange with endogenous IgG4 antibodies, while retaining the low affinity for activating Fc receptors associated with wild-type IgG4 antibodies (Wang et al., 2014). In yet other embodiments, the antibody comprises a light chain constant region which is a human kappa or lambda constant region. In other embodiments, the PD-1/PD-L1 axis antagonist antibody or antigen-binding portion thereof is a monoclonal antibody or an antigen-binding portion thereof. In certain embodiments of any of the therapeutic methods described herein comprising administration of an PD-1/PD-L1 axis antagonist antibody, the antibody is nivolumab. In other embodiments, the antibody is pembrolizumab. In other embodiments, the PD-1 antagonist antibody is chosen from the human antibodies 17D8, 2D3, 4H1, 4A11, 7D3 and 5F⁴ described in U.S. Pat. No. 8,008,449. In still other embodiments, the PD-1 antagonist antibody is MEDI0608 (formerly AMP-514), AMP-224, or Pidilizumab (CT-O11).

In certain embodiments, the antibody to be administered with the CD73 antagonist antibody is an anti-PD-L1 antibody. Because anti-PD-1 and anti-PD-L1 antibodies target the same signaling pathway and have been shown in clinical trials to exhibit similar levels of efficacy in a variety of cancers, an anti-PD-L1 antibody can be substituted for the PD-1 antagonist antibody in any of the therapeutic methods or compositions disclosed herein. In one embodiment, the anti-PD-L1 antibody is BMS-936559 (formerly 12A4 or MDX-1105) (see, e.g., U.S. Pat. No. 7,943,743; WO 2013/173223), or an antibody that comprises the CDRs or variable regions of 3G10, 12A4, 10A5, 5F8, 10H10, 1B12, 7H1, 11E6, 12B7 and 13G4, which are described in PCT Publication WO 07/005874 and U.S. Pat. No. 7,943,743. In certain embodiment, an anti-PD-L1 antibody is MEDI4736 (also known as Anti-B7-H1), MPDL3280A (also known as RG7446, atezolizumab and TECENTRIQ), MSB0010718C (W2013/79174), or rHigM12B7. Any of the anti-PD-L1 antibodies disclosed in WO2013/173223, WO2011/066389, WO2012/145493, U.S. Pat. Nos. 7,635,757 and 8,217,149 and U.S. Publication No. 2009/145493 may also be used. Anti-PD-L1 antibodies that compete with and/or bind to the same epitope as that of any of these antibodies may also be used in the treatments described herein.

Thus, generally, a PD-1/PD-L1 axis antagonist agent that may be used in the methods described herein include nivolumab, pembrolizumab, atelozilumab, durvalumab, REGN2810, PDR001, AMP-514 (MEDI0608), AMP-224, BGB-A317 or a PD-1 or PD-L1 antagonist described in any one of the following publications: WO 2009/014708, WO 03/099196, WO 2009/114335 and WO 2011/161699.

### IV. Pharmaceutical Compositions

Further provided are compositions, e.g., a pharmaceutical compositions, containing a CD73 antagonist antibody formulated alone or together with a PD-1/PD-L1 axis antagonist antibody, and a pharmaceutically acceptable carrier.

In certain embodiments, a composition comprises a CD73 antagonist antibody at a concentration of at least 1 mg/ml, 5 mg/ml, 10 mg/ml, 50 mg/ml, 100 mg/ml, 150 mg/ml, 200 mg/ml, 1-300 mg/ml, or 100-300 mg/ml.

Pharmaceutical compositions described herein also can be administered in combination therapy, i.e., combined with other agents. For example, the combination therapy can include a CD73 antagonist antibody described herein combined with a PD-1/PD-L1 axis antagonist antibody.

In some embodiments, therapeutic compositions disclosed herein can include other compounds, drugs, and/or agents used for the treatment of cancer. Such compounds, drugs, and/or agents can include, for example, chemotherapy drugs, small molecule drugs or antibodies that stimulate the immune response to a given cancer. In some instances, therapeutic compositions can include, for example, one or more of the agents listed in the section on combination therapies.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., antibody, immunoconjugate, orbispecific molecule, may be coated in a material to protect the compound from the action of acids and other natural conditions that may inactivate the compound.

The pharmaceutical compounds described herein may include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine, and the like.

A pharmaceutical composition described herein also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions described herein is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms described herein are dictated by and directly dependent on (a) the unique characteristics of the active compound and the particular therapeutic effect to be achieved, and (b) the limitations inherent in the art of compounding such an active compound for the treatment of sensitivity in individuals.

An antibody can be administered as a sustained release formulation, in which case less frequent administration is required. Dosage and frequency vary depending on the half-life of the antibody in the patient. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Actual dosage levels of the active ingredients in the pharmaceutical compositions described herein may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions described herein employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective dosage” of a CD73 antagonist antibody described herein preferably results in a decrease in severity of disease symptoms, an increase in frequency and duration of disease symptom-free periods, or a prevention of impairment or disability due to the disease affliction. In the context of cancer, a therapeutically effective dose preferably prevents further deterioration of physical symptoms associated with cancer. Symptoms of cancer are well-known in the art and include, for example, unusual mole features, a change in the appearance of a mole, including asymmetry, border, color and/or diameter, a newly pigmented skin area, an abnormal mole, darkened area under nail, breast lumps, nipple changes, breast cysts, breast pain, death, weight loss, weakness, excessive fatigue, difficulty eating, loss of appetite, chronic cough, worsening breathlessness, coughing up blood, blood in the urine, blood in stool, nausea, vomiting, liver metastases, lung metastases, bone metastases, abdominal fullness, bloating, fluid in peritoneal cavity, vaginal bleeding, constipation, abdominal distension, perforation of colon, acute peritonitis (infection, fever, pain), pain, vomiting blood, heavy sweating, fever, high blood pressure, anemia, diarrhea, jaundice, dizziness, chills, muscle spasms, colon metastases, lung metastases, bladder metastases, liver metastases, bone metastases, kidney metastases, and pancreatic metastases, difficulty swallowing, and the like.

A therapeutically effective dose may prevent or delay onset of cancer, such as may be desired when early or preliminary signs of the disease are present. Laboratory tests utilized in the diagnosis of cancer include chemistries (including the measurement of CD73 levels), hematology, serology, and radiology. Accordingly, any clinical or biochemical assay that monitors any of the foregoing may be used to determine whether a particular treatment is a therapeutically effective dose for treating cancer. One of ordinary skill in the art would be able to determine such amounts based on such factors as the subject's size, the severity of the subject's symptoms, and the particular composition or route of administration selected.

A composition described herein can be administered via one or more routes of administration using one or more of a variety of methods known in the art. As will be appreciated by the skilled artisan, the route and/or mode of administration will vary depending upon the desired results. Preferred routes of administration for antibodies described herein include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Alternatively, an antibody described herein can be administered via a non-parenteral route, such as a topical, epidermal or mucosal route of administration, for example, intranasally, orally, vaginally, rectally, sublingually, or topically.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Therapeutic compositions can be administered with medical devices known in the art. For example, in a preferred embodiment, a therapeutic composition described herein can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules for use with CD73 antagonist antibodies described herein include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In certain embodiments, the CD73 antagonist antibodies described herein can be formulated to ensure proper distribution in vivo. For example, the blood-brain barrier (BBB) excludes many highly hydrophilic compounds. To ensure that the therapeutic compounds described herein cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J. Physiol. 1233:134); p 120 (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273.

### V. Kits and Unit Dosage Forms

Also provided herein are kits which include a pharmaceutical composition containing a CD73 antagonist antibody (e.g., CD73.A) and a PD-1/PD-L1 axis antagonist antibody (e.g., nivolumab), and a pharmaceutically-acceptable carrier, in a therapeutically effective amount adapted for use in the preceding methods. The kits optionally also can include instructions, e.g., comprising administration schedules, to allow a practitioner (e.g., a physician, nurse, or patient) to administer the composition contained therein to administer the composition to a patient having cancer (e.g., a solid tumor). The kit also can include a syringe.

Optionally, the kits include multiple packages of the single-dose pharmaceutical compositions each containing an effective amount of the CD73 antagonist antibody or PD-1/PD-L1 axis antagonist antibody for a single administration in accordance with the methods provided above. Instruments or devices necessary for administering the pharmaceutical composition(s) also may be included in the kits. For instance, a kit may provide one or more pre-filled syringes containing an amount of the CD73 antagonist antibody or PD-1/PD-L1 axis antagonist antibody.

Accordingly, in one embodiment, provided herein is a kit for treating a solid tumor in a human patient, the kit comprising:

(a) a dose of a CD73 antagonist antibody comprising CDR1, CDR2, and CDR3 domains of the heavy chain variable region having the sequence set forth in SEQ ID NO: 6, and CDR1, CDR2, and CDR3 domains of the light chain variable region having the sequence set forth in SEQ ID NO: 7;

(b) a dose of a PD-1/PD-L1 axis antagonist antibody comprising CDR1, CDR2, and CDR3 domains of the heavy chain variable region having the sequence set forth in SEQ ID NO: 18, and CDR1, CDR2, and CDR3 domains of the light chain variable region having the sequence set forth in SEQ ID NO: 19; and

(c) instructions for using the CD73 antagonist antibody and PD-1/PD-L1 axis antagonist antibody in the methods described herein.

The present disclosure is further illustrated by the following examples, which should not be construed as further limiting. The contents of all figures and all references, Genbank sequences, patents and published patent applications cited throughout this application are expressly incorporated herein by reference. In particular, the disclosures of PCT publications WO09/045957, WO09/073533, WO09/073546, WO09/054863, WO2014/089113, WO2016/075099, WO2016/055609, WO2016/081748, WO 2017/152085, and U.S. Patent Publication Nos. 2011/0150892 and 2016/129108 are expressly incorporated herein by reference.

### EXAMPLES

### Example 1: Phase I Clinical Trial with a CD73 Antagonist Antibody

This Example describes the clinical trial of a CD73 antibody, CD73.A. CD73.A is an antibody comprising two heavy chains, each consisting of SEQ ID NO: 3 or 4, and two light chains, each consisting of SEQ ID NO: 5, which antibody was previously described in WO16/081748 and WO17/152085, the entire contents of which are specifically incorporated by reference herein. Briefly, CD73.A potently binds human CD73, and has a dual mechanism of action: inhibiting its enzymatic activity and promoting internalization (Barnhart B C, et al. Cancer Res. 2016; 76 (14 suppl) (abstract 1476)). Table 2 provides a summary of characteristics of CD73.A:

TABLE 2
Characteristics of CD73.A

| | | | Recombinant | Cellular Enzyme | |
|---|---|---|---|---|---|
| | | | Human | Inhibition | |
| | CD73 Binding | | CD73 | (blockade | Inter- |
| | in Cell Lines | | Enzyme | of AMP | nal- |
| | Human | Cyno | Inhibition | processing) | ization |
| Median | 0.5 | 0.3 | 2.97 | 0.39 | 1.2 |
| EC50, nM | | | | | |
| Range | 0.3-0.67 | 0.1-0.5 | 2.9-3.1 | 0.31-0.48 | — |
| Max level | — | — | — | — | 97.5% |

The clinical trial aimed to assess the safety and tumor-shrinking ability of experimental medication CD73.A alone and when combined with nivolumab (a PD-1 antagonist antibody), in patients with solid cancers that are advanced or have spread. The intervention included CD73.A administered intravenously in a “monotherapy lead-in”, followed by a combination of CD73.A and nivolumab. A dose expansion study is ongoing.

Primary outcome measures were the number of adverse events (AEs), serious adverse events (SAEs), AEs leading to discontinuation, and death. Secondary outcome measures included the following:
CD73.A enzyme assays in pre- and on-treatment biopsies
CD73.A immunohistochemistry (IHC) in pre- and on-treatment biopsies
Objective response rate (ORR), duration of response (DOR), progression free survival rate (PFSR)
Maximum observed serum concentration (Cmax), time of maximum observed serum concentration (Tmax), area under the serum concentration-time curve from time zero to time of the last quantifiable concentration [AUC(0-T)], area under the serum concentration-time curve in 1 dosing interval [AUC(TAU)], apparent terminal half-life (T-HALF), area under the serum concentration-time curve from time zero extrapolated to infinite time [AUC(INF)], effective elimination half-life (T-HALFeff)
Concentration at the end of the dosing interval (Ctau), trough observed serum concentration at the end of the dosing interval (Ctrough), total body clearance (CLT), volume of distribution at steady state (Vss), accumulation index (AI), apparent volume of distribution of terminal phase (Vz), degree of fluctuation or fluctuation index (DF)
Frequency of positive anti-drug antibody (ADA) to CD73.A and nivolumab

Inclusion criteria were: at least 18 years of age, advanced solid tumors, Eastern Cooperative Oncology Group (ECOG) 0-1, acceptable lab test results, and allow biopsies.

Exclusion criteria were: patients with central nervous system (CNS) tumors, uncontrolled or significant cardiovascular diseases, active or known autoimmune disease, or organ transplant.

Detailed inclusion criteria:
Aged at least 18 years at time of informed consent.
Eastern Cooperative Oncology Group (ECOG) performance status of ≤1.
At least one lesion with measurable disease as defined by RECIST v1.1
Prior exposure to therapy with any agent specifically targeting checkpoint pathway inhibition (such as anti-PD-1, anti-PD-L1, anti-PD-L2, anti-LAG-3, and anti-CTLA-4 antibody) after a washout period of any time greater than 4 weeks from the last treatment (Note: (i) Subjects who experienced prior Grade 1 to 2 checkpoint therapy-related immune-mediated AEs must have confirmed recovery from these events, other than endocrinopathies treated with supplementation, as documented by resolution of all related clinical symptoms, abnormal findings on physical examination, and/or associated laboratory abnormalities. Where applicable, these subjects must also have completed steroid tapers for treatment of these AEs by a minimum of 14 days prior to commencing treatment with study drug. (ii) Eligibility of subjects with prior ≥Grade 3 checkpoint therapy-related immune AEs may be eligible (e.g., asymptomatic isolated Grade 3 lipase elevations without clinical or radiological features of pancreatitis are permitted).
Prior therapy with any agent specifically targeting T-cell co-stimulation pathways such as anti-glucocorticoid induced tumor necrosis factor receptor, anti-CD137, or anti-OX40 antibody, with exceptions listed below, after a washout period of any time greater than 4 weeks from the last treatment.
Prior palliative radiotherapy must have been completed at least 2 weeks prior to first dose of the study drug. Subjects with symptomatic tumor lesions at baseline that may require palliative radiotherapy within 4 weeks of first dose of study drug should receive palliative radiotherapy prior to treatment.
Consent to pre-treatment tumor biopsy and have accessible lesions.
Adequate organ function, defined as follows:
(i) White blood cell count ≥2000/μL (stable off any growth factor within 2 weeks of the first study drug administration); (ii) Neutrophils ≥1500/μL (stable off any growth factor within 2 weeks of the first study drug administration); (iii) Platelets ≥100×10³/μL (transfusion to achieve this level is not permitted within 2 weeks of the first study drug administration); (iv) Hemoglobin ≥9 g/dL (transfusion to achieve this level is not permitted within 2 weeks of the first study drug administration); (v) Alanine aminotransferase (ALT) and aspartate aminotransferase (AST)≤3× the upper limit of normal (ULN); (vi) Total bilirubin ≤1.5×ULN (except subjects with Gilbert's Syndrome who must have normal direct bilirubin); (vii) Albumin >2 g/dL (20 g/L); (viii) International normalized ratio <1.5×ULN, activated partial thromboplastin time <1.5×ULN; (ix) Clinically normal thyroid function or have controlled hypothyroidism on appropriate thyroid supplementation; (x) Serum creatinine ≤1.5×ULN or creatinine clearance (CrCl)≥40 mL/min (measured using the Cockcroft-Gault formula below):

$$Female CrC1 = \frac{( 140 - age in years ) \times weight in kg \times 0.85}{72 \times serum creatinine in mg \text{/} dL}$$

$$Male CrC1 = \frac{( 140 - age in years ) \times weight in kg \times 1.00}{72 \times serum creatinine in mg \text{/} dL}$$

Ability to comply with treatment, PK, immunogenicity, biomarker, and PD sample collection, and required study follow-up.
Women of childbearing potential (WOCBP) must have a negative serum or urine pregnancy test (urine pregnancy test minimum sensitivity 25 IU/L or equivalent units of human chorionic gonadotropin [HCG]) within 24 hours prior to the start of the study drug.
Not breastfeeding.
Received and then progressed or been intolerant to, at least 1 standard treatment regimen in the advanced or metastatic setting.
All solid tumor histologies are permitted except primary CNS tumors or with CNS metastases as the only site of active disease.
For ovarian cancer, (a) received and progressed/been intolerant of at least 1 prior platinum-containing treatment regimen, and (b) sensitive to platinum and received at least 2 prior platinum-containing lines of treatment.
For CRC, (a) received and progressed/been intolerant of at least 1 standard systemic therapy for metastatic and/or unresectable disease (or have progressed within 6 months of adjuvant therapy), and (b) known KRAS mutation status.
For gastric cancer (including gastroesophageal junction tumors), received and progressed/been intolerant of at least 1 prior standard systemic therapy for metastatic and/or unresectable disease (or have progressed within 6 months of adjuvant therapy).
For pancreatic cancer, received and progressed/been intolerant of (or not be a candidate for) at least 1 prior standard therapy.
   Exclusion criteria were as follows:

### Target Disease Exceptions:

Known or suspected CNS metastases, untreated CNS metastases, or with the CNS as the only site of disease. However, controlled brain metastases are allowed. Controlled brain metastases are defined as no radiographic progression for at least 4 weeks following radiation and/or surgical treatment (or 4 weeks of observation if no intervention is clinically indicated), off of steroids for at least 2 weeks, and no new or progressive neurological signs and symptoms.
Carcinomatous meningitis.
Participation in any prior clinical study with nivolumab in which OS is listed as the primary or co-primary endpoint and which has not completed analysis based on the primary endpoint.
For pancreatic cancer: clinically relevant ascites at baseline (defined as requiring paracentesis) or with moderate radiographic ascites. Only a minimal amount of radiographic ascites is allowed.

### Medical History and Concurrent Diseases

Prior malignancy, different from the one used for enrollment, diagnosed less than 2 years prior (except non-melanoma skin cancers and in situ cancers such as the following: bladder, colon, cervical/dysplasia, melanoma, or breast). Subjects with second malignancies diagnosed more than 2 years ago who have received therapy with curative intent with no evidence of disease during the interval and who present a low risk for recurrence are eligible.
Other active malignancy requiring concurrent intervention.
Prior organ allograft.
Subjects who have received prior anti-cancer treatments are permitted (i.e., chemotherapy, radiotherapy, hormonal, or immunotherapy): for cytotoxic agents, at least 4 weeks must have elapsed from last dose of prior anti-cancer therapy and the initiation of study therapy; for non-cytotoxic agents, at least 4 weeks or 5 half-lives (whichever is shorter) must have elapsed from last dose of prior anti-cancer therapy and the initiation of study therapy.
Received prior therapy with an anti-CD73 antibody, an anti-CD39 antibody, or an adenosine 2A receptor inhibitor.
Prior history of cerebrovascular accident, deep vein thrombosis, or other arterial thrombus within the last 6 months.
Active, known or suspected autoimmune disease, with the following exceptions. Subjects with vitiligo, Type 1 diabetes mellitus, residual hypothyroidism due to autoimmune condition only requiring hormone replacement, subjects with euthyroid with a history of Grave's disease (subjects with suspected autoimmune thyroid disorders must be negative for thyroglobulin and thyroid peroxidase antibodies and thyroid-stimulating immunoglobulin prior to the first dose of the study drug), psoriasis not requiring systemic treatment, or conditions not expected to recur in the absence of an external trigger are permitted.
Interstitial lung disease that is symptomatic or may interfere with the detection or management of suspected drug-related pulmonary toxicity.
Chronic obstructive pulmonary disease requiring recurrent steroids bursts or chronic steroids at doses greater than 10 mg/day of prednisone or the equivalent.
Condition requiring systemic treatment with either corticosteroids (>10 mg daily prednisone equivalents) or other immunosuppressive medications within 14 days of study drug administration except for adrenal replacement steroid doses >10 mg daily prednisone equivalent in the absence of active autoimmune disease. Note: Treatment with a short course of steroids (<5 days) up to 7 days prior to initiating study drug is permitted.
Uncontrolled or significant cardiovascular disease including, but not limited to, any of the following:
(i) Myocardial infarction or stroke/transient ischemic attack within the past 6 months, (ii) Uncontrolled angina within the past 3 months; (iii) Any history of clinically significant arrhythmias (such as ventricular tachycardia, ventricular fibrillation, or torsades de pointes), (iv) QT interval corrected for heart rate using Fridericia's formula (QTcF) prolongation >480 msec; (v) History of other clinically significant heart disease (e.g., cardiomyopathy, congestive heart failure with New York Heart Association [NYHA] functional Classification III to IV, pericarditis, significant pericardial effusion); (vi) Requirement for daily supplemental oxygen therapy
Active hepatitis as evidenced by the following:
(i) Positive test for hepatitis B surface antigen; (ii) Positive test for hepatitis C antibody and/or qualitative viral load (by polymerase chain reaction [PCR]) (Note: Subjects with positive hepatitis C antibody and negative quantitative hepatitis C by PCR are eligible. History of resolved hepatitis A virus infection is not an exclusion criterion.)
Evidence of active bacterial, viral, or fungal infections 7 days prior to initiation of study drug therapy.
Known history of testing positive for human immunodeficiency virus (HIV) or known acquired immunodeficiency syndrome (AIDS).
Evidence or history of active or latent tuberculosis infection including purified protein derivative recently converted to positive, chest x-ray with evidence of infectious infiltrate, or recent unexplained changes in fever/chill patterns.
Major surgery within 4 weeks of study drug administration. Subjects must have recovered from the effects of major surgery or significant traumatic injury at least 14 days before the first dose of the study drug.
All toxicities attributed to prior anti-cancer therapy other than alopecia and fatigue must have resolved to Grade 1 (National Cancer Institute [NCI] Common Terminology Criteria for Adverse Events [CTCAE] Version 4.03) or baseline before administration of the study drug. Subjects with toxicities attributed to prior anti-cancer therapy that are not expected to resolve and result in long-lasting sequelae, such as chronic neuropathy after platinum based therapy, are permitted.
Use of non-oncology vaccines containing live virus for prevention of infectious diseases within 12 weeks prior to study drug. The use of inactivated seasonal influenza vaccines, eg, Fluzone®, are permitted without restriction.
Used packed red blood cells or received platelet transfusion within 2 weeks prior to the first dose of the study drug.
Known or underlying medical condition that could make administration of study drug hazardous, or could adversely affect the ability to comply with or tolerate the study.

Allergies and Adverse Drug Reaction
History of allergy to nivolumab are excluded.
History of any significant drug allergy (such as anaphylaxis) to prior anti-cancer immune modulating therapies (e.g., checkpoint inhibitors, T-cell co-stimulatory antibodies) are excluded.

Results from the clinical trial are described in the following Examples.

### Example 2: Preliminary Phase 1 Profile of a CD73 Antagonist Antibody, CD73.A, in Combination with Nivolumab, in Patients with Advanced Solid Tumors

CD73 is an ectonuclease that converts adenosine monophosphate into adenosine, a potent immunosuppressive soluble mediator that inhibits the cytotoxic function of CD8+ T cells and natural killer cells while promoting proliferation of immunosuppressive cells. CD73.A is a high affinity antibody which inhibits CD73 enzymatic activity and downregulates its expression on tumor cells. Blockade of CD73 enhanced the antitumor activity of anti-PD-1 in preclinical models (Barnhart B C, et al. Cancer Res. 2016; 76 (14 Suppl). Abstract 1476). Here, preliminary results of the first-in-human phase 1/2a study of CD73.A+nivolumab in patients with advanced solid tumors (NCT02754141) are provided.

Patients with ≥1 prior therapy were treated in this open-label, dose-escalation and -expansion study. Escalation began with a 2-week monotherapy lead-in where patients received CD73.A 150-1600 mg (150 mg, 300 mg, 600 mg, 1200 mg, and 1600 mg) IV Q1W, followed by CD73.A (same dose) Q1W+NIVO 240 mg Q2W (IV). Pharmacokinetics (PK), pharmacodynamics (PD), safety, and preliminary antitumor activity were evaluated. PD analyses included immunohistochemistry, enzyme activity assays in tumor biopsies, and evaluation of receptor occupancy and soluble CD73 in peripheral blood.

As of the Dec. 19, 2017 data cutoff, 59 patients were treated with CD73.A±nivolumab during dose escalation. The PK of CD73.A was non-linear at lower doses due to target-mediated drug disposition (TMDD), and exposure increased proportionally from 1200-1600 mg. CD73.A demonstrated complete and persistent CD73 target engagement in the periphery and tumor at all doses. Both monotherapy lead-in and the combination were well tolerated with no G4 treatment-related AEs (TRAEs) and no treatment-related deaths. TRAEs were observed in 30 of 52 patients (58%) who received the combination, with no clear dose relationship. Only 8 patients (15%) experienced G3 TRAEs and 1 discontinued treatment due to a TRAE (G3 increased ALT). CD73.A efficiently inhibited CD73 enzyme activity in the tumor vasculature and tumor cells at all doses without dose dependency. Overall, 7 patients with head and neck, pancreatic, prostate, anal, and renal cancer achieved confirmed partial responses and 10 patients had stable disease. Four responses occurred beyond 19 weeks, later than typical for nivolumab, suggesting preliminary clinical activity. Responses were also observed in tumor types not typically sensitive to nivolumab.

CD73.A+nivolumab was well tolerated, with CD73 target engagement in the periphery and tumor, and with a safety profile similar to NIVO monotherapy. The combination demonstrated preliminary antitumor activity, including clinical benefit among patients who received prior IO and those with tumors not typically sensitive to anti-PD-1 therapies.

### Example 3: CD73 is Expressed in Tumors

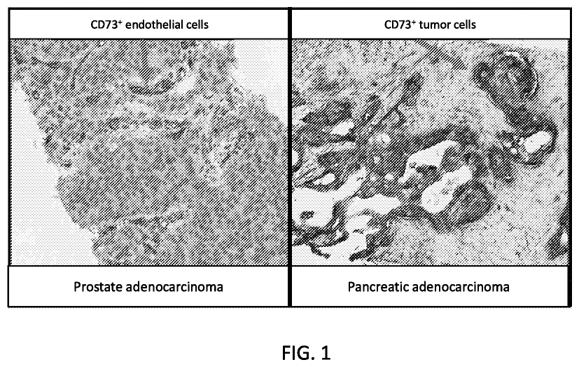

Tumor tissue from two cancer patients enrolled in the clinical trial was stained with a CD73 antagonist antibody. The results, which are shown in FIG. 1, indicate that prostate adenocarcinoma contains CD73-positive endothelial cells and pancreatic adenocarcinoma contains CD73-positive tumor cells.

### Example 4: Details of Clinical Trial and Safety Profile of Enrolled Patients

The clinical trial is a Phase 1/2a, open-label study of CD73.A administered as a single agent and in combination with nivolumab in subjects with advanced solid tumors, and is conducted in 3 parts: Part 1A (combination therapy dose escalation with monotherapy lead-in), Part 1B (PD substudy), and Part 2 (cohort expansion). Patients previously treated with immune checkpoint inhibitors were included in the trial.

As discussed above, the primary objective of the clinical trial was to assess the safety and tolerability of CD73.A administered alone and the combination of CD73.A and nivolumab. To this end, the number of adverse events (AEs), serious adverse events (SAEs), AEs leading to discontinuation, and deaths were assessed.

Secondary objectives of the trial were to characterize the pharmacodynamic (PD) activity of CD73.A administered alone and in combination with nivolumab; assess the preliminary anti-tumor activity of CD73.A in combination with nivolumab, as measured by objective response rate (ORR), duration of response (DOR), and progression-free survival rate (PFSR); to characterize the pharmacokinetics (PK) and immunogenicity of CD73.A administered alone and in combination with nivolumab; and to characterize immunogenicity of nivolumab when administered in combination with CD73.A.

Exploratory objectives of the trial were to explore associations of PD activity with efficacy and safety outcomes; to explore the exposure-response relationship for PD activity; to assess overall survival (OS) in subjects treated with CD73.A in combination with nivolumab; to characterize the PK of nivolumab when administered in combination with CD73.A; to characterize the dose-limiting toxicity (DLT) profile of CD73.A administered alone or in combination with nivolumab; and to capture Bayesian analysis of toxicity.

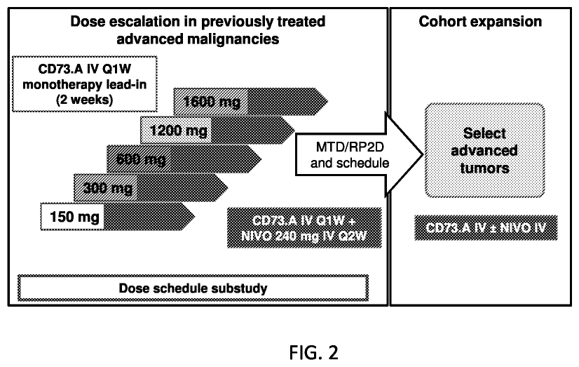

A dose escalation study was conducted in patients with previously treated advanced malignancies. The design of the dose escalation and cohort expansion is shown in FIG. 2. Briefly, patients received the assigned dose of CD73.A (150 mg-1600 mg; 150 mg, 300 mg, 600 mg, 1200 mg, and 1600 mg) on Day 1 of Cycle 0 (14-day cycle in monotherapy lead-in) on a Q1W schedule. Beginning on Day 1 of Cycle 1 (28-day cycle combination therapy for 6 cycles), patients were administered nivolumab at a flat dose of 240 mg Q2W in addition to the weekly doses of CD73.A, with the nivolumab dosing regimen being the same at each CD73.A dose level.

The baseline demographics, tumor type, and prior therapy of the patients enrolled in the trial are shown in Table 3. Demographics were similar across dose levels.

TABLE 3
Demographics, tumor type, and prior therapy of enrolled patients
CD73.A Q1W ± nivolumab 240 mg Q2W

| | | Total (N = 59) | |
|---|---|---|---|
| Median age (range), years | | 60.0 | (35-77) |
| Gender, n (%) | Male | 34 | (58) |
| ECOG PS, n (%) | 0 | 24 | (41) |
| | 1 | 35 | (59) |
| Race, n (%) | White | 55 | (93) |
| | Black | 2 | (3) |
| | Asian^{a} | 2 | (3) |
| Tumor type, n (%) | Renal cell carcinoma | 9 | (15) |
| | Colorectal cancer | 8 | (14) |
| | Pancreatic cancer | 7 | (12) |
| | Head and neck cancer | 5 | (8) |
| | Prostate cancer | 4 | (7) |
| | Ovarian cancer | 3 | (5) |
| | Other^{b} | 23 | (39) |
| No of prior systemic | 1 | 34 | (58) |
| therapies, n (%) | 2 | 7 | (12) |
| | 3 | 5 | (8) |
| | ≥4 | 11 | (19) |
| | Not reported | 2 | (3) |
| Prior immunotherapy, | Prior immunotherapy, | 12 | (20) |
| n (%) | n (%) | | |
| | Anti-PD-1/anti-PD-L1 | 6 | (10) |
| | Other checkpoint | 4 | (7) |
| | inhibitor | | |
| | Other 1-0 | 4 | (7) |

CRC = colorectal; ECOG PS = Eastern Cooperative Oncology Group performance status; RCC = renal cell carcinoma; SCCHN = squamous cell carcinoma of the head and neck
^{a}Includes Asian Indian and Asian other;
^{b}Includes tumors reported in ≤2 patients: anal canal epidermoid (n = 2), breast (n = 2), endometrial (n = 2), gastroesophageal junction (n = 2), hepatocellular, (n = 2), melanoma (n = 2), alveolar soft part (n = 1), cervical (n = 1), cholangiocarcinoma (n = 1), esophagus (n = 1), gastric (n = 1), intrahepatic cholangiocarcinoma (n = 1), leiomyosarcoma (n =1), Merkel cell (n = 1), squamous cell anorectal (n = 1), squamous cell carcinoma of the tongue (n = 1), and urothelial (n = 1);
^{c}Patients may have had >1 prior immuno-oncology therapy

The safety summary of enrolled patients is shown in Table 4. There was n clear dose relationship with TRAEs and the maximum tolerated dose was not reached.

TABLE 4

| | CD73.A | | | | CD73.A Q1W + Nivolumab 240 mg Q2W | | | | | | | | | | | | | |
|---|---|---|---|---|---|---|---|---|---|---|---|---|---|---|---|---|---|---|
| | Q1W | | | | CD73.A | | CD73.A | | CD73.A | | CD73.A | | CD73.A | | | | | |
| | Total | | | | 150 mg | | 300 mg | | 600 mg | | 1200 mg | | 1600 mg | | Total | | | |
| | (n = 59) | | | | (n = 12) | | (n = 11) | | (n = 12) | | (n = 7) | | (n = 10) | | (n = 52) | | | |
| | Any, n | | G3, n | | Any, | G3, | Any, | G3, | Any, | G3, | Any, | G3, | Any, | G3, | Any, n | | G3, n | |
| | (%) | | (%) | | n | n | n | n | n | n | n | n | n | n | (%) | | (%) | |
| Any TRAE | 23 | (39) | 1 | (2) | 7 | 2^{a,b} | 7 | 2 | 5 | 3^{a,c} | 5 | 0 | 6 | 1^{a,d} | 30 | (58) | 8 | (15)^{a-d} |
| TRAEs in | | | | | | | | | | | | | | | | | | |
| ≥5% of pts | | | | | | | | | | | | | | | | | | |
| Headache | 5 | (8) | 0 | | 0 | 0 | 0 | 0 | 0 | 0 | 0 | 0 | 0 | 0 | 0 | | 0 | |
| Fatigue | 2 | (3) | 0 | | 2 | 0 | 2 | 1 | 1 | 0 | 1 | 0 | 0 | 0 | 6 | (12) | 1 | (2) |
| Hypothyroidism | 0 | | 0 | | 2 | 0 | 2 | 0 | 0 | 0 | 0 | 0 | 0 | 0 | 4 | (8) | 0 | |
| Increased lipase | 0 | | 0 | | 0 | 0 | 1 | 1 | 2 | 2 | 0 | 0 | 1 | 0 | 4 | (8) | 3 | (6) |
| Increased ALT | 1 | (2) | 1 | (2) | 1 | 0 | 0 | 0 | 1 | 1 | 1 | 0 | 0 | 0 | 3 | (6) | 1 | (2) |

| Increased amylase | 0 | | 0 | 0 | 0 | 1 | 0 | 1 | 0 | 0 | 0 | 1 | 0 | 3 | (6) | 0 |
|---|---|---|---|---|---|---|---|---|---|---|---|---|---|---|---|---|
| Diarrhea | 2 | (3) | 0 | 1 | 0 | 0 | 0 | 0 | 0 | 1 | 0 | 1 | 0 | 3 | (6) | 0 |
| Periorbital edema | 1 | (2) | 0 | 1 | 0 | 1 | 0 | 1 | 0 | 0 | 0 | 0 | 0 | 3 | (6) | 0 |
| Pruritus | 2 | (3) | 0 | 0 | 0 | 3 | 0 | 0 | 0 | 0 | 0 | 0 | 0 | 3 | (6) | 0 |
| Pyrexia | 1 | (2) | 0 | 1 | 0 | 1 | 0 | 0 | 0 | 1 | 0 | 0 | 0 | 3 | (6) | 0 |

| | | | | | | | | | | | | | | | | |
|---|---|---|---|---|---|---|---|---|---|---|---|---|---|---|---|---|
| TRAEs leading | 0 | 0 | 2 | 1 | 0 | 0 | 1 | 1 | 0 | 0 | 0 | 0 | 3 | (6) | 2 | (4) |

to DC

^{a}Other grade 3 TRAEs included ^{b}adrenal insufficiency and increased transaminases (150 mg, n = 1 each), ^{c}autoimmune hepatitis and hepatitis (600 mg, n = 1), and ^{d}pancreatitis (1600 mg, n = 1) ^{e}Total patients treated with CD73.A ± nivolumab during dose escalation as of the Feb. 20, 2018 data cutoff; mono = monotherapy; combo = combination therapy

### Example 5. Pharmacokinetics of CD73.A in Human Patients

This Example describes the pharmacokinetics (PK) of CD73.A administered to patients enrolled in the clinical trial.

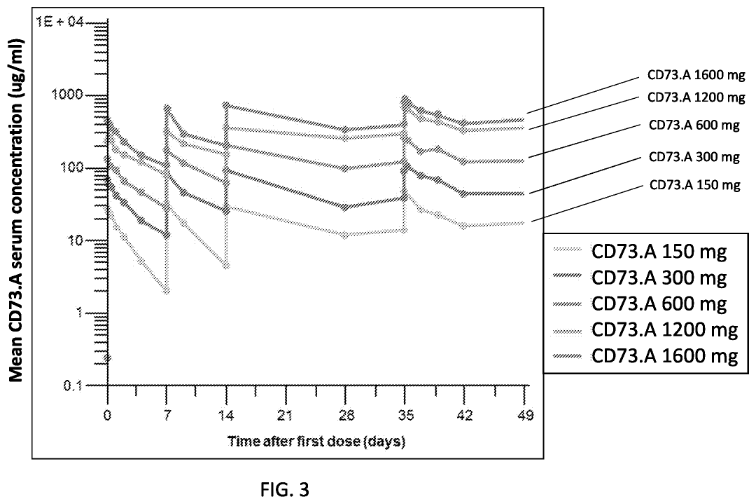

As shown in FIG. 3, the PK of CD73.A appears to be non-linear at lower doses due to target-mediated drug disposition (TMDD). Exposure of the antibody increased proportionally at higher doses, with steady-state concentrations reached in about 4 to 5 weeks.

### Example 6. Receptor Occupancy and Cell Surface Levels of CD73 in Patients Administered CD73.A

This Example describes the peripheral target engagement of CD73.A in peripheral cells in patients of the clinical trial. Receptor occupancy was determined as described in Example 22 of WO 2017/152085, incorporated herein by reference in its entirety and for all purposes. This receptor occupancy assay allows the detection of CD73.A antibody even in the presence of nivolumab.

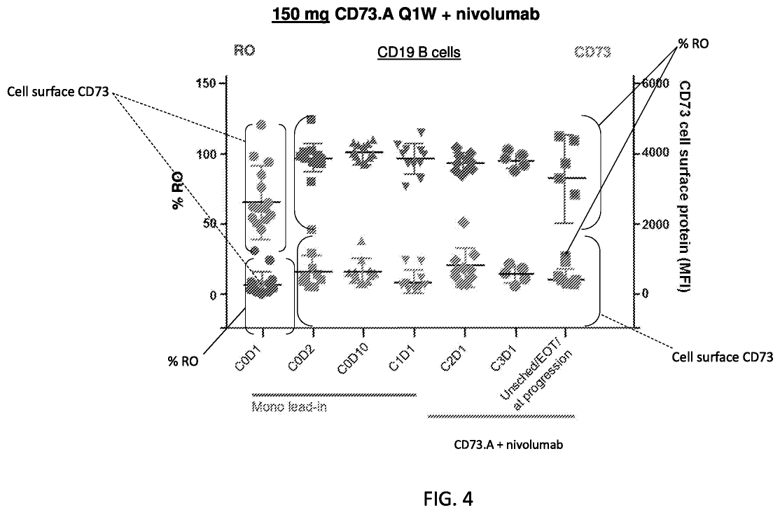

As shown in FIG. 4, CD73.A at 150 mg Q1W led to rapid and complete receptor occupancy and internalization of CD73 on the surface of CD19 B cells within 24 hours of administering CD73.A. The effect was consistent across all dose levels, and persisted until at least 30 days after administration of the CD73 antibody.

### Example 7: Levels of Free Soluble CD73 in Patients Administered CD73.A

This Example describes the effects of CD73.A on free (i.e., not bound by CD73.A) soluble CD73 (sCD73) levels in peripheral blood of patients of the clinical trial.

The free sCD73 assay utilizes biotinylated anti-CD73 antibody 6E11 (SEQ ID NOs: 28 and 29) to capture drug-unbound sCD73 from the test samples (as it competes with CD73.A for binding to hCD73). MSD Streptavidin Gold assay plates were coated overnight at 2 to 8° C. with 50 μL/well of biotinylated 6E11 diluted to a final concentration of 0.5 μg/mL in 1× DPBS buffer. The following day, the wells were washed using PBS with 0.05% Tween 20 and 0.5× StabilCoat prepared in water was added to wells at 100 μL per well and incubated at room temperature for one hour. Wells were then aspirated and plates were dried and pouched with desiccant. Test samples drawn from study animals were kept frozen at −70° C. On the day of assay, the test samples were thawed, mixed well, and diluted to the minimum required dilution (MRD) 1:2 in Starting Block (PBS). Standard curve calibrators prepared on the day of each run in Starting Block (PBS) were used to define the dynamic range of the bioanalytical method using the reference standard hCD73-his. Quality control samples, prepared earlier and stored at −70° C., were thawed on the day of sample analysis and processed in the same manner as the test samples. Prepared samples, quality controls (QCs) and calibrators were incubated on the coated plate for one hour at 25° C. with shaking, at about 500 rpm, to allow binding of free sCD73 to the capture reagent on the plate. Unbound material from samples were washed from the wells. Bound sCD73 was subsequently detected using ruthenylated anti-CD73 antibody 4C3 (SEQ ID NOs: 38 and 39) (previously prepared with 0.1% Proclin 300 at working concentration) at a final concentration of 0.125 μg/ml in Starting Block (PBS) at 50 μL per well. The plates were incubated for one hour at 25° C. with shaking, at about 500 rpm, and then washed again before adding 1× MSD Read Buffer prepared in water and then read on MSD Sector Imager 600 by electrochemiluminescenece (ECL) technology. The technical range of the standard curve was 160-0.039 ng/mL. Test samples were quantified using a 4-parameter logistic fit regression model.

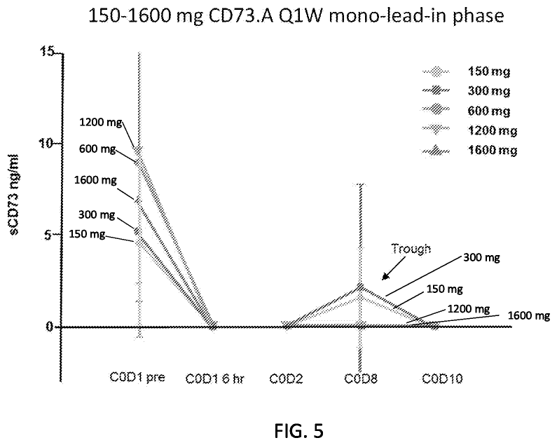

As shown in FIG. 5, free soluble CD73 was undetectable as early as 6 hours after administering CD73.A at all doses tested, and remained undetectable at all time points assessed at doses ≥600 mg (rebound effect on Day 8 at doses <600 mg).

### Example 8: CD73 Enzymatic Activity in Patients Administered CD73.A

This Example describes the effects of CD73.A on CD73 enzymatic activity in patients of the clinical trial. CD73 enzymatic activity was measured using the following protocol, adapted from: Aliagas et al., 2014. High Expression of Ecto-Nucleotidases CD39 and CD73 in Human Endometrial Tumors. Mediators of Inflammation. http://www.hindawi.com/journals/mi/2014/509027/.

1. Section OCT embedded tissues at 5 m, and dry for at least 10 mins
2. Fix sections in acetone for 10 mins (do not rinse), and dry for at least 10 mins
3. Store slides with a desiccant at −80° C.
4. When ready to use, acclimate slides at −20° C. for 20 mins before bringing to room temperature (about 25° C.)
5. Post-fix sections for 2 mins in 10% NBF (neutral buffered formalin), and wash slides 3× with PBS
6. Incubate sections in Solution 1 for 15 mins at room temp
7. After 15 mins in solution 1 (50 mM Tris-Maleate, 2 mM CaC2, 250 mM Sucrose, pH 7.4) tap off Solution 1 (do not rinse)
8. Incubate sections in Solution 2 (Solution 1 containing 5 mM MnCl2, 2 mM Pb(NO3)2, 2.5% w/v Dextran T200, 2.5 mM Levamisole, 1 mM AMP) for 60 mins) at 37° C. Negative control: Solution 2 without AMP
9. After 60 mins (one hour) in Solution 2, wash sections 1× in PBS
10. Incubate slides in room temperature 1% v/v (NH4)2S for exactly 2 mins
11. Rinse slides several times with dH2O
12. Counterstain (e.g., Mayer's Hematoxylin 30 secs, tap water rinse, blueing agent 3 mins, tap water rinse)
13. Dehydrate in graded alcohols & xylene, coverslip with a xylene-based mounting medium.

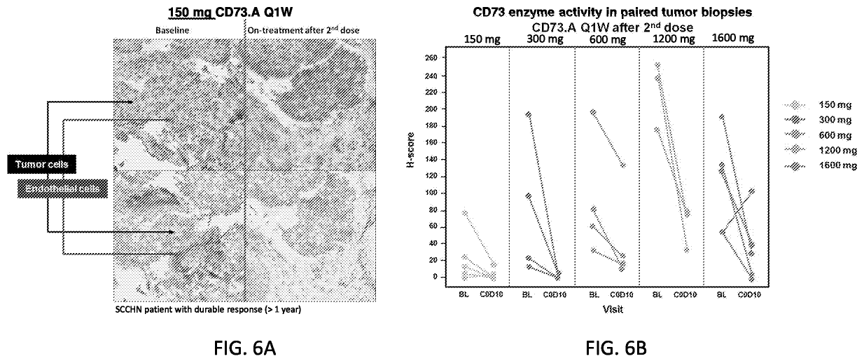

As shown in FIGS. 6A and 6B, CD73.A efficiently inhibited CD73 enzyme activity in both the tumor vasculature (endothelial cells) and tumor cells. The inhibition was apparent at all doses tested on Day 10 of the CD73.A monotherapy lead-in (FIG. 6B) and was maintained for at least 30 days post administration of the CD73 antibody.

### Example 9: Case Report of Patient with Prostate Carcinoma

This Example describes a 66-year-old male patient with prostate carcinoma treated with the combination of CD73.A and nivolumab. The carcinoma was microsatellite stable, with a Gleason score of 7. The patient had undergone prior treatment with enzalutamide, triptorelin, testosterone, docetaxel, and cabazitaxel.

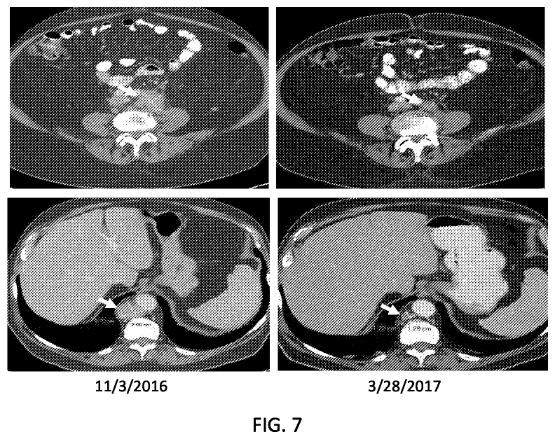

A partial response was achieved by treatment with CD73.A 300 mg Q1W and nivolumab 240 mg Q2W (prostate-specific antigen: baseline 692; nadir <0.1; current 0.1) (FIG. 7). The response was achieved by week 10 of the combination treatment, and was sustained until week 35. The patient had a solitary spinal metastasis, which was treated by resection and external radiotherapy. The best reduction in target lesion tumor burden was 75%. The combination treatment was resumed post-progression and is ongoing at week 68+.

### Example 10: Case Report of Patient with Gastroesophageal Junction Carcinoma

This Example describes a 61-year-old male patient with gastroesophageal junction carcinoma (adenocarcinoma of the gastroesophageal junction) treated with the combination of CD73.A and nivolumab. The carcinoma was microsatellite stable and HER2 negative. The patient had undergone prior treatment with FOLFOX and paclitaxel+ramucirumab.

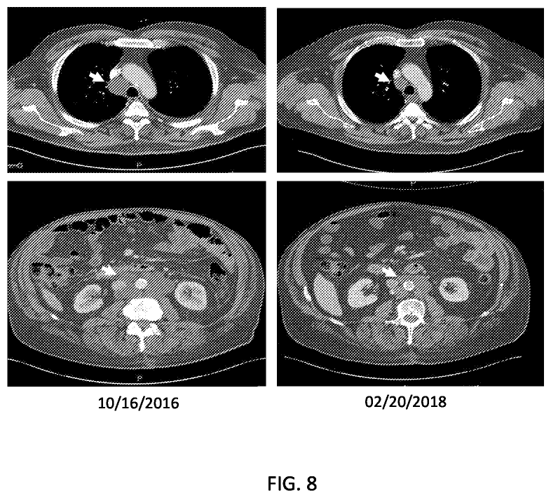

A partial response was achieved by treatment with CD73.A 600 mg and nivolumab 240 mg (FIG. 8). The response was achieved by week 59. The best reduction in target lesion tumor burden was 36%. Tumor reduction is ongoing and the patient continues to be on treatment at week 67+.

### Example 11: Selection of Recommended Phase 2 Dose

Table 5 summarizes the results described in the preceding Examples, including safety, TMDD saturation, peripheral target coverage, and enzyme inhibition in tumors.

TABLE 5
CD73.A Q1W ± nivolumab 240 mg Q2W

| | Threshold dose level |
|---|---|
| TMDD saturation | ≥600 mg |
| Peripheral target coverage | |
| Target engagement (RO) | ≥150 mg |
| CD73 internalization | ≥150 mg |
| Free sCD73 saturation (C0D8 rebound) | ≥600 mg |
| Tumor enzyme inhibition (EHC) | ≥150 mg |

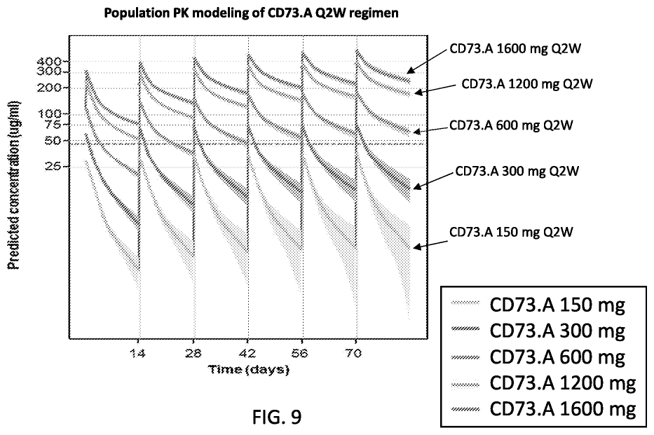

In summary, tolerability, tumor CD73 inhibition, and clinical benefit were seen at all dose levels. Durable saturation of sCD73 was observed at ≥600 mg CD73.A Q1W. Steady state trough concentrations of CD73.A were observed at 600 mg Q1W, and predicted concentrations at Q2W exceed the TMDD threshold (FIG. 9). Based on these results and population PK modeling of CD73.A Q2W regimen, CD73.A 600 mg Q2W+nivolumab 240 mg Q2W (or nivolumab 480 mg Q4W) was selected as the recommended phase 2 dose.

### Example 12: Quantitative Analysis of Total sCD73 in Human Serum as a Pharmacodynamic (PD) Biomarker

Total sCD73 was quantitatively measured in human serum of patients by immunocapture-LC-MS/MS as follows. Briefly, a sensitive immunocapture LC-MS/MS assay was developed to quantify total sCD73 as a PD biomarker. A surrogate matrix approach was used for the quantification of the endogenous protein. A non-competing antibody (4C3) was utilized to capture both free and drug-bound CD73. Parallelism was established during assay qualification. Several bioanalytical challenges for endogenous compounds had to be overcome: the endogenous nature of analyte, e.g., the presence of endogenous protein in the matrix and potential difference in the form and structure from the recombinant protein; high sensitivity requirement; specificity and selectivity requirement; parallelism needs to be demonstrated that dilution of test samples does not result in biased measurements of the analyte concentration between endogenous and recombinant protein and surrogate matrix and sample matrix.

Chemicals, Reagents, Materials and Apparatus: Formic Acid (SupraPur grade) was purchased from EMD Chemicals (Gibbstown, N.J., USA). HPLC grade methanol was purchased from J. T. Baker (Phillipsburg, N.J., USA). LC grade acetonitrile, ammonium bicarbonate and phosphate buffered saline with 0.05% tween (PBST) were purchased from Sigma-Aldrich (St. Louis, Mo., USA). Dynabeads® M-280 Streptavidin was purchased from Invitrogen (Carlsbad, Calif., USA). Sequencing grade modified trypsin was purchased from Promega Corporation (Madison, Wis., USA). The stable isotope-labeled surrogate peptide internal standards (SIL-VIYPAVEGR) was synthesized by from Genscript (Piscataway, N.J., USA). Deionized water was generated in house using a NANOpure Diamond ultrapure water system from Barnstead International (Dubuque, Iowa, USA). Mouse serum was obtained from Bioreclamation, Inc. (Westbury, N.Y., USA). Recombinant human CD73 (clone 4C3; SEQ ID NOs: 38 and 39) and the anti-human CD73 monoclonal antibody (mAb) were generated internally at BMS.

Equipment and Apparatus: The LC-MS system used was a triple quadrupole 5500 mass spectrometer (AB Sciex, Foster City, Calif.), coupled with Nexera UHPLC system (Shimadzu, Columbia, Md., USA), which consists of two LC-30AD pumps, two DGU-20A5 degassers, one SIL-30ACMP autosampler and one CTO-30AS column heater. Data acquisition was controlled by Analyst® 1.6.2 software. Trypsin digestion was carried out on the thermomixer R (model 5355) & MTP Microblock (Eppendorf, Hamburg, Germany).

Preparation of calibration standards and quality control samples: Recombinant human CD73 reference material was received as a 1.3 mg/mL solution in buffer containing PBS pH7.4/50 μM ZnCl2. The solution was sub-aliquoted into single-use vials and stored at −70° C. for long term use. Mouse serum was used as a surrogate matrix for preparation of calibration standards and quality control samples (QCs). The calibration standard curve range is established from 1.00 to 500 ng/mL. QC samples were prepared at the concentrations of 1.00, 3.00, 25.0, 250, 400 and 20,000 ng/mL. After preparation, the standards and QCs were stored at −70° C. before analysis. The stock solution of the stable isotope-labeled internal standard (SIL-VIYPAVEGR) was prepared at a concentration of 0.5 mg/mL in 50:50 (v:v) water:acetonitrile. The stock solution was further diluted to a final concentration of 10 ng/mL with 4% Formic acid in 50:50 (v:v) water:acetonitrile and used as the internal standard working solution.

Biotinylation: Biotinylation of the capture antibody was performed using EZ-Link® Sulfo-NHS-LC-Biotin (Thermo Scientific catalog #21327) according to manufacturer's instructions with a 20:1 challenge molar ratio. Biotinylated antibody was then purified using Zeba™ spin desalting columns (Thermo Scientific catalog #89893).

Immunocapture and Trypsin Digestion: Magnetic beads preparation: Dynabeads® M-280 Streptavidin bead (10 mg/mL) was first washed three times with PBST. The anti human CD73 mAb 4C3 biotinated mAb was immobilized to the washed bead suspension at a 200 μg antibody per mL of bead ratio. The solution was then incubated at room temperature for 30 min, then washed three times with PBST.

Sample preparation: An aliquot of 100 μL of standards, QCs and unknown samples were transferred to a 96-well protein LoBind plate (Eppendorf, Hauppauge, N.Y.), and diluted by 100 μL of PBST buffer. A volume of 25 μL of the prepared magnetic bead suspension as described above was then added. The mixture was incubated at room temperature for 1 hr on the thermomixer and washed 2 times with PBST. The captured analyte was then eluted using 100 μL of 12 mM HCl. The eluents were collected in a clean LoBind plate and neutralized with 10 μL of 100 mM ammonium bicarbonate.

Digestion: Thermo denature was first conducted by incubating the eluted samples at 90° C. for 30 min. The plate was then cooled down to room temperature, followed by the addition of 10 μl of Promega trypsin (100 μg/mL in 100 mM ammonium bicarbonate, total 1 μg per sample) and incubated at 37° C. overnight. After incubation, the digestion was stopped by the addition of 10 μl of the internal standard working solution (10 ng/mL of SIL-VIYPAVEGR prepared in 4% formic acid in 50:50 (v:v) water:acetonitrile). The samples were vortex mixed and centrifuged at 3000 rpm for 5 min before analysis.

HPLC-MS/MS Conditions: The mobile phase A contains 0.005% formic acid in water and mobile phase B contains 0.005% formic acid in methanol. The HPLC separation was carried out on an Acquity UPLC BEH C18 column (1.7 μm, 2.1×50 mm from Waters) with the column temperature set to maintain 60° C. Gradient elution was conducted at the following gradient: mobile phase B was maintained at 15% from 0 to 0.5 min; and increased to 70% from 0.5 to 3 min. Then the B % was linearly increased to 95% in 0.1 min and was held for 0.5 min; and then decreased to 15% in 0.1 min. The flow rate was set as 0.6 mL/min and the total run time was 4 min.

The digested peptides were monitored with selective reaction monitoring (SRM) using positive ion electrospray ionization (ESI) with the following optimized MS conditions: curtain gas and collision gas were set as 30 and 8; the turbo spray voltage was set at 3500 V and ion source gas 1 and gas 2 were both set at 50 psi. The probe temperature was set at 600° C. and entrance Potential (EP) was maintained at 10 V. For the SRM detection, doubly charged molecular ion for peptide VIYPAVEGR (C-terminal peptide of hCD73) was selected at Q1 and the SRM transitions monitored for VIYPAVEGR and SIL-VIYPAVEGR were 502.6/628.3 and 506.0/628.3 with declustering potential (DP) set at 60 V and collision energy (CE) at 25 eV.

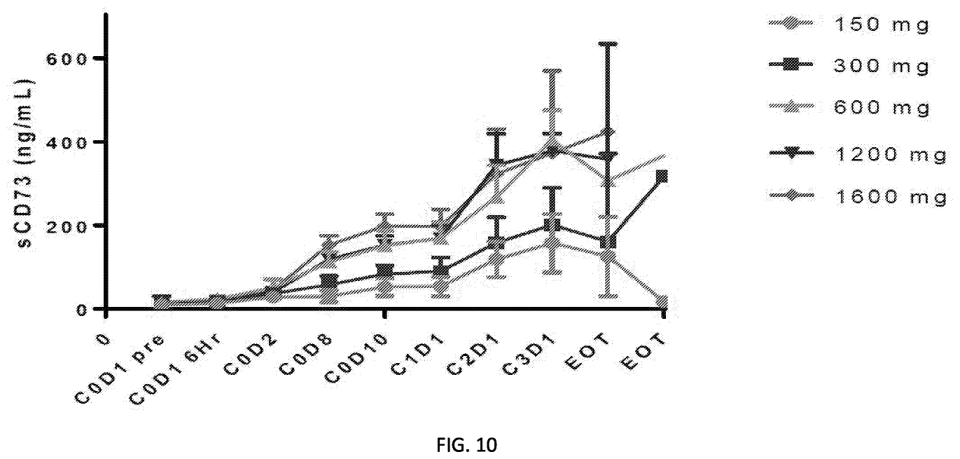

The results, which are shown in FIG. 10, indicate that total sCD73 levels increase during treatment with CD73.A, but goes down at the end of the treatment with lower doses.

Quantitative measurement of the soluble serum CD73 level will, inter alia, assist in dose selection and provide valuable pharmacodynamic information for clinical drug development, as well as be useful as a prognostic or predictive biomarker and can be determined prior to treatment (baseline biomarker) or after, e.g., a first dose of treatment.

### EQUIVALENTS

Those skilled in the art will recognize or be able to ascertain, using no more than routine experimentation, many equivalents of the specific embodiments described herein described herein. Such equivalents are intended to be encompassed by the following claims.

TABLE 6
Summary of sequences

| SEQ | | |
|---|---|---|
| ID | Description | Sequence |
| 1 | Human CD73 isoform 1 | MCPRAARAPATLLLALGAVLWPAAGAWELTILHTNDVH |
| | | SRLEQTSEDSSKCVNASRCMGGVARLFTKVQQIRRAEP |
| | | NVLLLDAGDQYQGTIWFTVYKGAEVAHFMNALRYDAMA |
| | | LGNHEFDNGVEGLIEPLLKEAKFPILSANIKAKGPLAS |
| | | QISGLYLPYKVLPVGDEVVGIVGYTSKETPFLSNPGTN |
| | | LVFEDEITALQPEVDKLKTLNVNKIIALGHSGFEMDKL |
| | | IAQKVRGVDVVVGGHSNTFLYTGNPPSKEVPAGKYPFI |
| | | VTSDDGRKVPVVQAYAFGKYLGYLKIEFDERGNVISSH |
| | | GNPILLNSSIPEDPSIKADINKWRIKLDNYSTQELGKT |
| | | IVYLDGSSQSCRFRECNMGNLICDAMINNNLRHTDEMF |
| | | WNHVSMCILNGGGIRSPIDERNNGTITWENLAAVLPFG |
| | | GTFDLVQLKGSTLKKAFEHSVHRYGQSTGEFLQVGGIH |
| | | VVYDLSRKPGDRVVKLDVLCTKCRVPSYDPLKMDEVYK |
| | | VILPNFLANGGDGFQMIKDELLRHDSGDQDINVVSTYI |
| | | SKMKVIYPAVEGRIKFSTGSHCHGSFSLIFLSLWAVIF |
| | | VLYQ |
| 2 | Human CD73 isoform 2 | MCPRAARAPATLLLALGAVLWPAAGAWELTILHTNDVH |
| | | SRLEQTSEDSSKCVNASRCMGGVARLFTKVQQIRRAEP |
| | | NVLLLDAGDQYQGTIWFTVYKGAEVAHFMNALRYDAMA |
| | | LGNHEFDNGVEGLIEPLLKEAKFPILSANIKAKGPLAS |
| | | QISGLYLPYKVLPVGDEVVGIVGYTSKETPFLSNPGTN |
| | | LVFEDEITALQPEVDKLKTLNVNKIIALGHSGFEMDKL |
| | | IAQKVRGVDVVVGGHSNTFLYTGNPPSKEVPAGKYPFI |
| | | VTSDDGRKVPVVQAYAFGKYLGYLKIEFDERGNVISSH |
| | | GNPILLNSSIPEDPSIKADINKWRIKLDNYSTQELGKT |
| | | IVYLDGSSQSCRFRECNMGNLICDAMINNNLRHTDEMF |
| | | WNHVSMCILNGGGIRSPIDERNNGIHVVYDLSRKPGDR |
| | | VVKLDVLCTKCRVPSYDPLKMDEVYKVILPNFLANGGD |
| | | GFQMIKDELLRHDSGDQDINVVSTYISKMKVIYPAVEG |
| | | RIKFSTGSHCHGSFSLIFLSLWAVIFVLYQ |
| 3 | Heavy chain-CD73.A | QVQLVESGGGVVQPGRSLRLSCAASGFTFSNYGMHWVR |
| | | QAPGKGLEWVAVILYDGSNKYYPDSVKGRFTISRDNSK |
| | | NTLYLQMNSLRAEDTAVYYCARGGSSWYPDSFDIWGQG |
| | | TMVTVSSASTKGPSVFPLAPCSRSTSESTAALGCLVKD |
| | | YFPEPVTVSWNSGALTSGVHTFPAVLQSSGLYSLSSVV |
| | | TVPSSNFGTQTYTCNVDHKPSNTKVDKTVERKSCVECP |
| | | PCPAPPVAGPSVFLFPPKPKDTLMISRTPEVTCVVVDV |
| | | SHEDPEVKFNWYVDGVEVHNAKTKPREEQYNSTYRVVS |
| | | VLTVLHQDWLNGKEYKCKVSNKALPSSIEKTISKAKGQ |
| | | PREPQVYTLPPSREEMTKNQVSLTCLVKGFYPSDIAVE |
| | | WESNGQPENNYKTTPPVLDSDGSFFLYSKLTVDKSRWQ |
| | | QGNVFSCSVMHEALHNHYTQKSLSLSPGK |
| 4 | Heavy chain-CD73.A, | QVQLVESGGGVVQPGRSLRLSCAASGFTFSNYGMHWVR |
| | without C-terminal | QAPGKGLEWVAVILYDGSNKYYPDSVKGRFTISRDNSK |
| | lysine | NTLYLQMNSLRAEDTAVYYCARGGSSWYPDSFDIWGQG |
| | | TMVTVSSASTKGPSVFPLAPCSRSTSESTAALGCLVKD |
| | | YFPEPVTVSWNSGALTSGVHTFPAVLQSSGLYSLSSVV |
| | | TVPSSNFGTQTYTCNVDHKPSNTKVDKTVERKSCVECP |
| | | PCPAPPVAGPSVFLFPPKPKDTLMISRTPEVTCVVVDV |
| | | SHEDPEVKFNWYVDGVEVHNAKTKPREEQYNSTYRVVS |
| | | VLTVLHQDWLNGKEYKCKVSNKALPSSIEKTISKAKGQ |
| | | PREPQVYTLPPSREEMTKNQVSLTCLVKGFYPSDIAVE |
| | | WESNGQPENNYKTTPPVLDSDGSFFLYSKLTVDKSRWQ |
| | | QGNVFSCSVMHEALHNHYTQKSLSLSPG |
| 5 | Light chain-CD73.A | DIQMTQSPSSLSASVGDRVTITCRASQGISSWLAWYQQ |
| | | KPEKAPKSLIYAASSLQSGVPSRFSGSGSGTDFTLTIS |
| | | SLQPEDFATYYCQQYNSYPLTFGGGTKVEIKRTVAAPS |
| | | VFIFPPSDEQLKSGTASVVCLLNNFYPREAKVQWKVDN |
| | | ALQSGNSQESVTEQDSKDSTYSLSSTLTLSKADYEKHK |
| | | VYACEVTHQGLSSPVTKSFNRGEC |
| 6 | Heavy chain variable | QVQLVESGGGVVQPGRSLRLSCAASGFTFSNYGMHWVR |
| | region-CD73.A | QAPGKGLEWVAVILYDGSNKYYPDSVKGRFTISRDNSK |
| | | NTLYLQMNSLRAEDTAVYYCARGGSSWYPDSFDIWGQG |
| | | TMVTVSS |
| 7 | Light chain variable | DIQMTQSPSSLSASVGDRVTITCRASQGISSWLAWYQQ |
| | region-CD73.A | KPEKAPKSLIYAASSLQSGVPSRFSGSGSGTDFTLTIS |
| | | SLQPEDFATYYCQQYNSYPLTFGGGTKVEIK |
| 8 | HCDR1-CD73.A | NYGMH |
| 9 | HCDR2-CD73.A | VILYDGSNKYYPDSVKG |
| 10 | HCDR3-CD73.A | GGSSWYPDSFDI |
| 11 | LCDR1-CD73.A | RASQGISSWLA |
| 12 | LCDR2-CD73.A | AASSLQS |
| 13 | LCDR3-CD73.A | QQYNSYPLT |
| 14 | IgG2/IgG1 hybrid Fc | ASTKGPSVFPLAPCSRSTSESTAALGCLVKDYFPEPVT |
| | (IgG2C219S-IgG1.1f) | VSWNSGALTSGVHTFPAVLQSSGLYSLSSVVTVPSSNF |
| | | GTQTYTCNVDHKPSNTKVDKTVERKSCVECPPCPAPPV |
| | | AGPSVFLFPPKPKDTLMISRTPEVTCVVVDVSHEDPEV |
| | | KFNWYVDGVEVHNAKTKPREEQYNSTYRVVSVLTVLHQ |
| | | DWLNGKEYKCKVSNKALPSSIEKTISKAKGQPREPQVY |
| | | TLPPSREEMTKNQVSLTCLVKGFYPSDIAVEWESNGQP |
| | | ENNYKTTPPVLDSDGSFFLYSKLTVDKSRWQQGNVFSC |
| | | SVMHEALHNHYTQKSLSLSPGK |
| 15 | Heavy chain- | QVQLVESGGGVVQPGRSLRLDCKASGITFSNSGMHWVR |
| | nivolumab | QAPGKGLEWVAVIWYDGSKRYYADSVKGRFTISRDNSK |
| | | NTLFLQMNSLRAEDTAVYYCATNDDYWGQGTLVTVSSA |
| | | STKGPSVFPLAPCSRSTSESTAALGCLVKDYFPEPVTV |
| | | SWNSGALTSGVHTFPAVLQSSGLYSLSSVVTVPSSSLG |
| | | TKTYTCNVDHKPSNTKVDKRVESKYGPPCPPCPAPEFL |
| | | GGPSVFLFPPKPKDTLMISRTPEVTCVVVDVSQEDPEV |
| | | QFNWYVDGVEVHNAKTKPREEQFNSTYRVVSVLTVLHQ |
| | | DWLNGKEYKCKVSNKGLPSSIEKTISKAKGQPREPQVY |
| | | TLPPSQEEMTKNQVSLTCLVKGFYPSDIAVEWESNGQP |
| | | ENNYKTTPPVLDSDGSFFLYSRLTVDKSRWQEGNVFSC |
| | | SVMHEALHNHYTQKSLSLSLGK |
| 16 | Heavy chain- | QAPGKGLEWVAVIWYDGSKRYYADSVKGRFTISRDNSK |
| | nivolumab without | NTLFLQMNSLRAEDTAVYYCATNDDYWGQGTLVTVSSA |
| | C-terminal lysine | STKGPSVFPLAPCSRSTSESTAALGCLVKDYFPEPVTV |
| | | SWNSGALTSGVHTFPAVLQSSGLYSLSSVVTVPSSSLG |
| | | TKTYTCNVDHKPSNTKVDKRVESKYGPPCPPCPAPEFL |
| | | GGPSVFLFPPKPKDTLMISRTPEVTCVVVDVSQEDPEV |
| | | QFNWYVDGVEVHNAKTKPREEQFNSTYRVVSVLTVLHQ |
| | | DWLNGKEYKCKVSNKGLPSSIEKTISKAKGQPREPQVY |
| | | TLPPSQEEMTKNQVSLTCLVKGFYPSDIAVEWESNGQP |
| | | ENNYKTTPPVLDSDGSFFLYSRLTVDKSRWQEGNVFSc |
| | | SVMHEALHNHYTQKSLSLSLG |
| 17 | Light chain- | EIVLTQSPATLSLSPGERATLSCRASQSVSSYLAWYQQ |
| | nivolumab | KPGQAPRLLIYDASNRATGIPARFSGSGSGTDFTLTIS |
| | | SLEPEDFAVYYCQQSSNWPRTFGQGTKVEIKRTVAAPS |
| | | VFIFPPSDEQLKSGTASVVCLLNNFYPREAKVQWKVDN |
| | | ALQSGNSQESVTEQDSKDSTYSLSSTLTLSKADYEKHK |
| | | VYACEVTHQGLSSPVTKSFNRGEC |
| 18 | Heavy chain variable | QVQLVESGGGVVQPGRSLRLDCKASGITFSNSGMHWVR |
| | region-nivolumab | QAPGKGLEWVAVIWYDGSKRYYADSVKGRFTISRDNSK |
| | | NTLFLQMNSLRAEDTAVYYCATNDDYWGQGTLVTVSS |
| 19 | Light chain variable | EIVLTQSPATLSLSPGERATLSCRASQSVSSYLAWYQQ |
| | region-nivolumab | KPGQAPRLLIYDASNRATGIPARFSGSGSGTDFTLTIS |
| | | SLEPEDFAVYYCQQSSNWPRTFGQGTKVEIK |
| 20 | HCDR1-nivolumab | NSGMH |
| 21 | HCDR2-nivolumab | VIWYDGSKRYYADSVKG |
| 22 | HCDR3-nivolumab | NDDY |
| 23 | LCDR1-nivolumab | RASQSVSSYLA |
| 24 | LCDR2-nivolumab | DASNRAT |
| 25 | LCDR3-nivolumab | QQSSNWPRT |
| 26 | CD73 epitope | FTKVQQIRRAEPNVLLLDA |
| 27 | CD73 epitope | LYLPYKVLPVGDEVVG |
| 28 | Heavy chain-6E11 | EVQLVESGGALVQPGRSLRLSCAASGFTFDDYAMHWVR |
| | | QAPGKGLEWVSGITWNSGGIGYADSVKGRFTISRDNAK |
| | | NSLYLQMNSLRAEDTALYYCAKDRYYSSWLLFDNWGQG |
| | | ILVTVSSASTKGPSVFPLAPSSKSTSGGTAALGCLVKD |
| | | YFPEPVTVSWNSGALTSGVHTFPAVLQSSGLYSLSSVV |
| | | SSSTVPLGTQTYICNVNHKPSNTKVDKKVEPKSCDKTH |
| | | TCPPCPAPELLGGPSVFLFPPKPKDTLMISRTPEVTCV |
| | | VVDVSHEDPEVKFNWYVDGVEVHNAKTKPREEQYNSTY |
| | | RVVSVLTVLHQDWLNGKEYKCKVSNKALPAPIEKTISK |
| | | AKGQPREPQVYTLPPSRDELTKNQVSLTCLVKGFYPSD |
| | | IAVEWESNGQPENNYKTTPPVLDSDGSFFLYSKLTVDK |
| | | SRWQQGNVFSCSVMHEALHNHYTQKSLSLSPGK |
| 29 | Light chain-6E11 | EIVLTQSPGTLSLSPGERATLSCRASQSVSSSYLAWYQ |
| | | QKPGQAPRLLIYGASSRATGIPDRFSGSGSGTDFTLTI |
| | | SRLEPEDFAVYYCQHYGSSFTFGPGTKVDIKRTVAAPS |
| | | VFIFPPSDEQLKSGTASVVCLLNNFYPREAKVQWKVDN |
| | | ALQSGNSQESVTEQDSKDSTYSLSSTLTLSKADYEKHK |
| | | VYACEVTHQGLSSPVTKSFNRGEC |
| 30 | Heavy chain variable | EVQLVESGGALVQPGRSLRLSCAASGFTFDDYAMHWVR |
| | region-6E11 | QAPGKGLEWVSGITWNSGGIGYADSVKGRFTISRDNAK |
| | | NSLYLQMNSLRAEDTALYYCAKDRYYSSWLLFDNWGQG |
| | | ILVTVSS |
| 31 | Light chain variable | EIVLTQSPGTLSLSPGERATLSCRASQSVSSSYLAWYQ |
| | region-6E11 | QKPGQAPRLLIYGASSRATGIPDRFSGSGSGTDFTLTI |
| | | SRLEPEDFAVYYCQHYGSSFTFGPGTKVDIK |
| 32 | HCDR1-6E11 | DYAMH |
| 33 | HCDR2-6E11 | GITWNSGGIGYADSVKG |
| 34 | HCDR3-6E11 | DRYYSSWLLFDN |
| 35 | LCDR1-6E11 | RASQSVSSSYLA |
| 36 | LCDR2-6E11 | GASSRAT |
| 37 | LCDR3-6E11 | QHYGSSFT |
| 38 | Heavy chain-4C3 | EVQLVESGGGLVQPGRSLRLSCAASGFTFDDYAMHWVR |
| | | QAPGKGLEWVSGISWKSGSIGYADSVKGRFTISRDNAK |
| | | NSLYLQMNSLRAEDTALYYCVKGYYVILTGLDYWGQGT |
| | | LVTVSSASTKGPSVFPLAPSSKSTSGGTAALGCLVKDY |
| | | FPEPVTVSWNSGALTSGVHTFPAVLQSSGLYSLSSVVT |
| | | VPSSSLGTQTYICNVNHKPSNTKVDKKVEPKSCDKTHT |
| | | CPPCPAPELLGGPSVFLFPPKPKDTLMISRTPEVTCVV |
| | | VDVSHEDPEVKFNWYVDGVEVHNAKTKPREEQYNSTYR |
| | | VVSVLTVLHQDWLNGKEYKCKVSNKALPAPIEKTISKA |
| | | KGQPREPQVYTLPPSRDELTKNQVSLTCLVKGFYPSDI |
| | | AVEWESNGQPENNYKTTPPVLDSDGSFFLYSKLTVDKS |
| | | RWQQGNVFSCSVMHEALHNHYTQKSLSLSPGK |
| 39 | Light chain-4C3 | EIVLTQSPGTLSLSPGERATLSCRASQSVSSYLAWYQQ |
| | | KPGQAPRLLIYGASSRATGIPDRFSGSGSGTDFTLTIS |
| | | RLEPEDFAVYYCQQYGSSPLTFGGGTKVEIKRTVAAPS |
| | | VFIFPPSDEQLKSGTASVVCLLNNFYPREAKVQWKVDN |
| | | ALQSGNSQESVTEQDSKDSTYSLSSTLTLSKADYEKHK |
| | | VYACEVTHQGLSSPVTKSFNRGEC |
| 40 | Heavy chain variable | EVQLVESGGGLVQPGRSLRLSCAASGFTFDDYAMHWVR |
| | region-4C3 | QAPGKGLEWVSGISWKSGSIGYADSVKGRFTISRDNAK |
| | | NSLYLQMNSLRAEDTALYYCVKGYYVILTGLDYWGQGT |
| | | LVTVSS |
| 41 | Light chain variable | EIVLTQSPGTLSLSPGERATLSCRASQSVSSYLAWYQQ |
| | region-4C3 | KPGQAPRLLIYGASSRATGIPDRFSGSGSGTDFTLTIS |
| | | RLEPEDFAVYYCQQYGSSPLTFGGGTKVEIK |
| 42 | HCDR1-4C3 | DYAMH |
| 43 | HCDR2-4C3 | GISWKSGSIGYADSVKG |
| 44 | HCDR3-4C3 | GYYVILTGLDY |
| 45 | LCDR1-4C3 | RASQSVSSYLAW |
| 46 | LCDR2-4C3 | ASSRATG |
| 47 | LCDR3-4C3 | QYGSSPLT |

The Sequence Listing provides the sequences of the mature variable regions and heavy and light chains (i.e., sequences do not include signal peptides). The sequences of the heavy chains with C-terminal lysine, may also be used without that lysine (K), or without the GK.

We claim:

1. A method of treating a subject having cancer, comprising administering to the subject a therapeutically effective dose of a CD73 antagonist antibody, wherein the method results in one or more of the following:
(a) steady state serum concentration of the CD73 antagonist antibody is achieved 3, 4, 5, or 6 weeks after the first administration of the CD73 antagonist antibody;
(b) full receptor occupancy of the CD73 antagonist antibody, e.g., on peripheral B cells such as CD19 B cells, is achieved within 24 hours of the first administration of the CD73 antagonist antibody;
(c) full receptor occupancy of the CD73 antagonist antibody is sustained for at least 30 days after administration of the last dose of the CD73 antagonist antibody;
(d) undetectable cell surface levels of CD73 on peripheral B cells, e.g., CD19 B cells within 24 hours of the first administration of the CD73 antagonist antibody;
(e) undetectable cell surface levels of CD73 up to at least 30 days after administration of the last dose of the CD73 antagonist antibody;
(f) undetectable free soluble CD73 within 6 hours of the first administration of the CD73 antagonist antibody;
(g) undetectable free soluble CD73 at the end of the last treatment cycle including the CD73 antagonist antibody; and
(h) decrease of CD73 enzyme activity in tumor cells and/or tumor vasculature compared to before administration of the CD73 antagonist antibody.

2. A method of treating a subject having cancer, comprising administering to the subject a therapeutically effective dose of a combination of a CD73 antagonist antibody and a PD-1/PD-L1 axis antagonist antibody, wherein the method results in one or more of the following:
(a) steady state serum concentration of the CD73 antagonist antibody is achieved 3, 4, 5, or 6 weeks after the first administration of the CD73 antagonist antibody;
(b) full receptor occupancy of the CD73 antagonist antibody, e.g., on peripheral B cells such as CD19 B cells, is achieved within 24 hours of the first administration of the CD73 antagonist antibody;
(c) full receptor occupancy of the CD73 antagonist antibody is sustained for at least 30 days after administration of the last dose of the CD73 antagonist antibody;
(d) undetectable cell surface levels of CD73 on peripheral B cells, e.g., CD19 B cells within 24 hours of the first administration of the CD73 antagonist antibody;
(e) undetectable cell surface levels of CD73 up to at least 30 days after administration of the last dose of the CD73 antagonist antibody;
(f) undetectable free soluble CD73 within 6 hours of the first administration of the CD73 antagonist antibody;
(g) undetectable free soluble CD73 at the end of the last treatment cycle including the CD73 antagonist antibody; and
(h) decrease of CD73 enzyme activity in tumor cells and/or tumor vasculature compared to before administration of the CD73 antagonist antibody.

3. A method of treating a subject having cancer, comprising administering to the subject a combination of CD73 antagonist antibody at a fixed dose of about 150-1600 mg once every week or once every two weeks and a PD-1/PD-L1 axis antagonist antibody at a fixed dose of 240 mg or about 240 mg once every two weeks or 480 mg or about 480 mg once every four weeks, wherein the method results in one or more of the following:
(a) steady state serum concentration of the CD73 antagonist antibody is achieved 3, 4, 5, or 6 weeks after the first administration of the CD73 antagonist antibody;
(b) full receptor occupancy of the CD73 antagonist antibody, e.g., on peripheral B cells such as CD19 B cells, is achieved within 24 hours of the first administration of the CD73 antagonist antibody;
(c) full receptor occupancy of the CD73 antagonist antibody is sustained for at least 30 days after administration of the last dose of the CD73 antagonist antibody;
(d) undetectable cell surface levels of CD73 on peripheral B cells, e.g., CD19 B cells within 24 hours of the first administration of the CD73 antagonist antibody;
(e) undetectable cell surface levels of CD73 up to at least 30 days after administration of the last dose of the CD73 antagonist antibody;
(f) undetectable free soluble CD73 within 6 hours of the first administration of the CD73 antagonist antibody;
(g) undetectable free soluble CD73 at the end of the last treatment cycle including the CD73 antagonist antibody; and
(h) decrease of CD73 enzyme activity in tumor cells and/or tumor vasculature compared to before administration of the CD73 antagonist antibody.

4. The method of any of claims 1-3, wherein the method comprises a CD73 antibody monotherapy lead in phase, wherein one or more (e.g., 1-3, 1-2, 1, 2, 3) doses of the CD73 antagonist antibody are administered within 1-3 weeks prior to the first dose of the PD-1/PD-L1 axis antagonist antibody.

5. The method of claim 4, wherein the one or more doses of the CD73 antagonist antibody are administered within the 2 weeks prior to the first dose of the PD-1/PD-L1 axis antagonist antibody.

6. The method of claim 5, wherein the CD73 antagonist antibody is administered Q1W in the CD73 antibody monotherapy lead-in.

7. The method of claim 5, wherein the CD73 antagonist antibody is administered Q2W in the CD73 antibody monotherapy lead-in.

8. The method of any of claims 4-6, wherein a first dose of the CD73 antagonist antibody is administered 2 weeks prior to the first dose of the PD-1/PD-L1 axis antagonist antibody, and optionally, a second dose of the CD73 antagonist antibody is administered 1 week prior to the first dose of the PD-1/PD-L1 axis antagonist antibody.

9. The method of any of claims 1-8, wherein the CD73 antibody and the PD-1/PD-L1 axis antagonist antibody are administered on the same day at least once.

10. The method of any of claims 1-9, wherein, when administered on the same day, the CD73 antagonist antibody and the PD-1/PD-L1 axis antagonist antibody are administered simultaneously at least once.

11. The method of claim 10, wherein, when administered on the same day, the CD73 antagonist antibody and the PD-1/PD-L1 axis antagonist antibody are administered sequentially at least once.

12. The method of any of claims 1-11, wherein combination treatment with the CD73 antagonist antibody and PD-1/PD-L1 axis antagonist antibody is on a 28-day cycle.

13. The method of claim 12, wherein the combination treatment consists of up to 6 cycles.

14. The method of any of claims 1-13, wherein the CD73 antagonist antibody is administered at a fixed dose of about 150 mg, 300 mg, 600 mg, 1200 mg, or 1600 mg.

15. The method of any of claims 1, 2, and 4-14, wherein the CD73 antagonist antibody is administered once a week, once every two weeks, once every three weeks, or once every four weeks.

16. The method of claim 15, wherein the CD73 antagonist antibody is administered once a week.

17. The method of claim 15, wherein the CD73 antagonist antibody is administered once every two weeks.

18. The method of any of claims 2 and 4-16, wherein the PD-1/PD-L1 axis antagonist antibody is administered once every two weeks or once every four weeks.

19. The method of claim 18, wherein the PD-1/PD-L1 axis antagonist antibody is administered once every two weeks at a fixed dose of about 240 mg.

20. The method of claim 18, wherein the PD-1/PD-L1 axis antagonist antibody is administered once every four weeks at a fixed dose of about 480 mg.

21. The method of any of claims 1-20, wherein the CD73 antagonist antibody and PD-1/PD-L1 axis antagonist antibody are formulated for intravenous administration.

22. The method of any of claims 1-20, wherein the CD73 antagonist antibody and PD-1/PD-L1 axis antagonist antibody are formulated for subcutaneous administration.

23. The method of claim 21 or 22, wherein the CD73 antagonist antibody and PD-1/PD-L1 axis antagonist antibody are formulated together.

24. The method of claim 21 or 22, wherein the CD73 antagonist antibody and PD-1/PD-L1 axis antagonist antibody are formulated separately.

25. The method of any of claims 1-24, wherein steady state serum concentration of the CD73 antagonist antibody is achieved 3, 4, 5, or 6 weeks after administration of the first dose of the CD73 antagonist antibody.

26. The method of any of claims 1-25, wherein target-mediated drug disposition (TMDD) saturation is achieved when the CD73 antagonist antibody is administered at a fixed dose of 600 mg or greater.

27. The method of any of claims 1-26, wherein full receptor occupancy of the CD73 antagonist antibody, e.g., on peripheral B cells such as CD19 B cells, is achieved within 24 hours of administration of the first dose of the CD73 antagonist antibody when the CD73 antagonist antibody is administered at a fixed dose of 150 mg or greater.

28. The method of any of claims 1-27, wherein full receptor occupancy of the CD73 antagonist antibody is sustained for at least 30 days after administering the last dose of the CD73 antagonist antibody when the CD73 antagonist antibody is administered at a fixed dose of 150 mg or greater.

29. The method of any of claims 1-28, wherein cell surface levels of CD73 on peripheral B cells, e.g., CD19 B cells, are undetectable within 24 hours of administration of the first dose of the CD73 antagonist antibody when the CD73 antagonist antibody is administered at a fixed dose of 150 mg or greater.

30. The method of any of claims 1-29, wherein cell surface levels of CD73 are undetectable for at least 30 days after administering the last dose of the CD73 antagonist when the CD73 antagonist antibody is administered at a fixed dose of 150 mg or greater.

31. The method of any of claims 1-30, wherein free soluble CD73 is undetectable within 6 hours of administration of the CD73 antagonist antibody when the CD73 antagonist antibody is administered at a fixed dose of 600 mg or greater.

32. The method of any of claims 1-31, wherein free soluble CD73 is undetectable at the end of the last treatment cycle of combination treatment with the CD73 antagonist antibody and PD-1/PD-L1 axis antagonist antibody or the end of the CD73 antibody monotherapy lead-in when the CD73 antagonist antibody is administered at a fixed dose of 600 mg or greater.

33. The method of any of claims 1-32, wherein CD73 enzyme activity is decreased in tumor cells and/or tumor vasculature compared to before administration of the CD73 antagonist antibody when the CD73 antagonist antibody is administered at a fixed dose of 150 mg or greater.

34. The method of any of claims 1-33, wherein the subject has received 1, 2, 3, or 4 or more prior therapies, e.g., systemic therapies.

35. The method of any of claims 1-34, wherein the subject has received one or more prior immunotherapies.

36. The method of claim 34 or 35, wherein the subject was refractory to the prior therapy.

37. The method of claim 35 or 36, wherein the one or more prior immunotherapies includes a PD-1 or PD-L1 axis antagonist therapy, e.g., with nivolumab.

38. The method of any of claims 1-37, wherein the subject progressed on or after prior cancer therapy.

39. The method of claim 38, wherein the subject progressed on or after a previous immunotherapy.

40. The method of claim 39, wherein the previous immunotherapy is a checkpoint inhibitor therapy.

41. The method of claim 40, wherein the checkpoint inhibitor therapy is a PD-1 or PD-L1 antagonist therapy.

42. The method of claim 39, wherein the previous immunotherapy is not a PD-1 or PD-L1 axis antagonist therapy.

43. The method of any of claims 1-42, wherein the method does not cause significant treatment-related adverse events, e.g., as determined in clinical trials.

44. The method of any of claims 1-43, wherein the cancer is an advanced solid tumor.

45. The method of claim 44, wherein the advanced solid tumor is not typically responsive to immunotherapy, e.g., not typically responsive to an anti-PD-1 or anti-PD-L1 antagonist.

46. The method of any of claims 1-45, wherein the cancer is selected from the group consisting of colorectal cancer, ovarian cancer, renal cell carcinoma, head and neck cancer, breast cancer, pancreatic cancer, prostate cancer, gastroesophageal cancer, hepatocellular carcinoma, melanoma, anal canal epidermoid carcinoma, endometrial cancer, gastric cancer, cervical cancer, gastroesophageal junction carcinoma, alveolar soft part carcinoma, cholangiocarcinoma, esophageal cancer, intrahepatic cholangiocarcinoma, leiomyosarcoma, Merkel cell carcinoma, squamous cell anorectal carcinoma, squamous cell carcinoma of the tongue, squamous cell carcinoma of the head and neck, and urothelial cancer.

47. The method of any of claims 1-46, wherein the cancer is microsatellite stable.

48. The method of any of claims 1-47, wherein the treatment produces at least one therapeutic effect chosen from a reduction in size of a tumor, reduction in number of metastatic lesions over time, complete response, partial response, and stable disease.

49. The method of any of claims 1-48, wherein the CD73 antagonist antibody comprises heavy chain variable region CDR1, CDR2, and CDR3 comprising the sequences set forth in SEQ ID NOs: 8, 9, and 10, respectively, and the light chain variable region CDR1, CDR2, and CDR3 comprising the sequences set forth in SEQ ID NOs: 11, 12, and 13, respectively.

50. The method of any of claims 1-49, wherein the CD73 antagonist antibody comprises heavy and light chain variable region sequences which are at least 80%, at least 85%, at least 90%, at least 95%, at least 98%, at least 99%, or 100% identical to the heavy and light chain variable region sequences set forth in SEQ ID NOs: 6 and 7, respectively.

51. The method of any of claims 1-50, wherein the CD73 antagonist antibody comprises heavy and light chain sequences which are at least 80%, at least 85%, at least 90%, at least 95%, at least 98%, at least 99%, or 100% identical to the heavy chain sequence set forth in SEQ ID NO: 3 or 4, and the light chain sequence set forth in SEQ ID NO: 5.

52. The method of any of claims 1-51, wherein the CD73 antagonist antibody is selected from the group consisting of an IgG1, an IgG2, an IgG3, an IgG4 or a variant or hybrid thereof.

53. The method of any of claims 1-52, wherein the Fc region of the CD73 antagonist antibody is an IgG2/IgG1 hybrid Fc region.

54. The method of claim 53, wherein the Fc region comprises the amino acid sequence set forth in SEQ ID NO: 14.

55. The method of any of claims 1-54, wherein the CD73 antagonist antibody is a human or humanized antibody.

56. The method of any of claims 1-55, wherein the PD-1/PD-L1 axis antagonist antibody comprises heavy chain variable region CDR1, CDR2, and CDR3 comprising the sequences set forth in SEQ ID NOs: 20, 21, and 22, respectively, and light chain variable region CDR1, CDR2, and CDR3 comprising the sequences set forth in SEQ ID NOs: 23, 24, and 25, respectively.

57. The method of any of claims 1-56, wherein the PD-1/PD-L1 axis antagonist antibody comprises heavy and light chain variable region sequences which are at least 80%, at least 85%, at least 90%, at least 95%, at least 98%, at least 99%, or 100% identical to the heavy and light chain variable region sequences set forth in SEQ ID NOs: 18 and 19, respectively.

58. The method of any of claims 1-57, wherein the PD-1/PD-L1 axis antagonist antibody comprises heavy and light chain sequences at least 80%, at least 85%, at least 90%, at least 95%, at least 98%, at least 99%, or 100% identical to the heavy chain sequence set forth in SEQ ID NO: 15 or 16, and the light chain sequence set forth in SEQ ID NO 17 (e.g., nivolumab).

59. A method of treating cancer, e.g., pancreatic cancer, in a human patient, the method comprising administering to the patient an effective amount of each of:
(a) a CD73 antagonist antibody comprising a heavy chain comprising the amino acid sequence set forth in SEQ ID NO: 3 or 4, and a light chain comprising the amino acid sequence set forth in SEQ ID NO: 5, and
(b) a PD-1 antagonist antibody comprising a heavy chain comprising the amino acid sequence set forth in SEQ ID NO: 15 or 16, and a light chain comprising the amino acid sequence set forth in SEQ ID NO: 17 (e.g., nivolumab),
wherein one or more (e.g., 1-3 or 1-2) doses of the CD73 antagonist antibody are administered within 1-3 weeks prior to the first dose of the PD-1/PD-L1 axis antagonist antibody, e.g., for one cycle, wherein one cycle is two weeks long, in a “CD73 antibody monotherapy lead-in,”
wherein, following the CD73 antibody monotherapy lead-in, the CD73 antagonist antibody is administered once a week at a fixed dose of about 150-1600 mg in combination with the PD-1 antagonist antibody, which is administered once every two weeks at a fixed dose of 240 mg or about 240 mg or once every four weeks at a fixed dose of 480 mg or about 480 mg, wherein the combination treatment consists, e.g., of up to six 28-day cycles.

60. A method of treating cancer, e.g., pancreatic cancer, in a human patient, the method comprising administering to the patient an effective amount of each of:
(a) a CD73 antagonist antibody comprising a heavy chain comprising the amino acid sequence set forth in SEQ ID NO: 3 or 4, and a light chain comprising the amino acid sequence set forth in SEQ ID NO: 5, and
(b) a PD-1 antagonist antibody comprising a heavy chain comprising the amino acid sequence set forth in SEQ ID NO: 15 or 16, and a light chain comprising the amino acid sequence set forth in SEQ ID NO: 17 (e.g., nivolumab),
wherein the CD73 antagonist antibody is administered once a week at a fixed dose of about 150-1600 mg in combination with the PD-1 antagonist antibody, which is administered once every two weeks at a fixed dose of 240 mg or about 240 mg or once every four weeks at a fixed dose of 480 mg or about 480 mg, wherein the combination treatment consists, e.g., of up to six 28-day cycles.

61. The method of claim 59 or 60, wherein the CD73 antagonist antibody is administered at a fixed dose of about 150 mg, 300 mg, 600 mg, 1200 mg, or 1600 mg.

62. A method of treating cancer, e.g., pancreatic cancer, in a human patient, the method comprising administering to the patient an effective amount of each of:
(a) a CD73 antagonist antibody comprising a heavy chain comprising the amino acid sequence set forth in SEQ ID NO: 3 or 4, and a light chain comprising the amino acid sequence set forth in SEQ ID NO: 5, and
(b) a PD-1 antagonist antibody comprising a heavy chain comprising the amino acid sequence set forth in SEQ ID NO: 15 or 16, and a light chain comprising the amino acid sequence set forth in SEQ ID NO: 17 (e.g., nivolumab),
wherein one or more (e.g., 1-3 or 1-2) doses of the CD73 antagonist antibody are administered within 1-3 weeks prior to the first dose of the PD-1/PD-L1 axis antagonist antibody, e.g., for one cycle, wherein one cycle is two weeks long, in a “CD73 antibody monotherapy lead-in,”
wherein, following the CD73 antibody monotherapy lead-in, the CD73 antagonist antibody is administered once every two weeks at a fixed dose of 600 mg or about 600 mg in combination with the PD-1 antagonist antibody which is administered once every two weeks at 240 mg or about 240 mg or once every four weeks at a fixed dose of 480 mg or about 480 mg, wherein the combination therapy consists, e.g., of up to six 28-day cycles.

63. A method of treating cancer, e.g., pancreatic cancer, in a human patient, the method comprising administering to the patient an effective amount of each of:
(a) a CD73 antagonist antibody comprising a heavy chain comprising the amino acid sequence set forth in SEQ ID NO: 3 or 4, and a light chain comprising the amino acid sequence set forth in SEQ ID NO: 5, and
(b) a PD-1 antagonist antibody comprising a heavy chain comprising the amino acid sequence set forth in SEQ ID NO: 15 or 16, and a light chain comprising the amino acid sequence set forth in SEQ ID NO: 17 (e.g., nivolumab),
wherein the CD73 antagonist antibody is administered once every two weeks at a fixed dose of 600 mg or about 600 mg in combination with the PD-1 antagonist antibody, which is administered once every two weeks at 240 mg or about 240 mg or once every four weeks at a fixed dose of 480 mg or about 480 mg, wherein the combination treatment consists, e.g., of up to six 28-day cycles.

64. The method of any of claims 59-63, wherein the patient has received one or more prior therapies to treat the cancer.

65. The method of claim 64, wherein the one or more prior therapies comprises one or more prior immunotherapies.

66. The method of claim 64 or 65, wherein the patient progressed on the one or more prior therapies.

67. The method of any of claims 2-66, wherein the method comprises first measuring the expression level of PD-L1, and if the expression level of PD-L1 is ≥1%, ≥5%, ≥10%, ≥25%, or ≥50%, e.g., as measured with, e.g., the PD-L1 IHC 28-8 pharmDx assay, then the subject is treated with the combination of the CD73 antagonist antibody and PD-1/PD-L1 axis antagonist antibody.

68. A method of treating cancer in a subject, comprising administering to a subject having been determined to have a peripheral level of free sCD73 protein (the sCD73 that is not bound by an anti-CD73 agent, e.g., that was administered to the subject) that is lower than that in a healthy subject, a therapeutically effective amount of an immunotherapy treatment (i.e., a treatment that stimulates the immune system).

69. A method of treating cancer in a subject, comprising administering to a subject having been determined to have a peripheral level of sCD73 protein (or free sCD73 protein in a subject having received a prior dose of an anti-CD73 agent) that is similar to (e.g., equal to) or higher than that in a healthy subject, a therapeutically effective amount of a CD73 antagonist and an immunotherapy treatment.

70. A method of treating cancer in a subject, comprising determining the level of free sCD73 in the peripheral blood of the subject, and
(i) if the level of free sCD73 protein is lower than that in a healthy subject, administer a therapeutically effective amount of an immunotherapy treatment; and
(ii) if the level of free sCD73 protein is similar to (e.g., equal to) or higher than that in a healthy subject, administer a therapeutically effective amount of a CD73 antagonist and optionally an immunotherapy treatment.

71. The method of claim 70, wherein, if the level of free sCD73 protein is similar to (e.g., equal to) or higher than that in a healthy subject, administer a therapeutically effective amount of a CD73 antagonist and an immunotherapy treatment.

72. The method of claim 71, wherein, if a CD73 antagonist and an immunotherapy treatment are administered, the CD73 antagonist is administered prior to the immunotherapy treatment, such that the level of free sCD73 protein has decreased to levels similar to or lower than those in a healthy subject, prior to administration of the immunotherapy treatment.

73. The method of claim 71 or 72, wherein the immunotherapy treatment is administered when the level of free sCD73 protein is decreased to a level that is at most 25%, 50%, 75%, 90% or 95% of the level of free sCD73 protein prior to administration of the CD73 antagonist.

74. The method of any of claims 71 to 73, wherein the first dose of the immunotherapy treatment is administered when the level of free sCD73 protein is decreased to a level that is at most 25%, 50%, 75%, 90% or 95% of the level of free sCD73 protein prior to administration of the CD73 antagonist.

75. The method of any of claims 71-74, wherein the first dose, or every dose of the immunotherapy treatment is administered when the level of free sCD73 protein is reduced to undetectable levels, e.g., as measured by a method described in the Examples.

76. The method of any of claims 71-75, wherein the CD73 antagonist is administered at least 6 hours prior to the immunotherapy treatment.

77. The method of claim 76, wherein the CD73 antagonist is administered at least 12 hours prior to the immunotherapy treatment.

78. A method of determining whether a subject having cancer will respond positively to an immunotherapy treatment, comprising determining the level of free sCD73 in the peripheral blood of the subject, and if the level of free sCD73 in the peripheral blood of the subject is similar to or lower than that in a healthy subject, then the subject is likely to respond positively to an immunotherapy treatment.

79. A method of determining whether a subject having cancer will respond positively to an immunotherapy treatment, comprising determining the level of free sCD73 in the peripheral blood of the subject, and if the level of free sCD73 in the peripheral blood of the subject is similar to or higher than that in a healthy subject, then the subject is not likely to respond positively to an immunotherapy treatment or is likely to respond to an immunotherapy treatment if the subject has a level of free sCD73 that is lower than that in a healthy subject.

80. A method of determining whether a subject having cancer should be treated with an immunotherapy treatment or an immunotherapy treatment together with an anti-CD73 therapeutic, comprising determining the level of free sCD73 in the peripheral blood of the subject, and
(i) if the level of free sCD73 in the peripheral blood of the subject is lower than that in a healthy subject, then administering to the subject an immunotherapy treatment; and
(ii) if the level of free sCD73 in the peripheral blood of the subject is similar to or higher than that in a healthy subject, then administering to the subject a CD73 antagonist and an immunotherapy treatment.

81. The method of any of claims 68-80, wherein the immunotherapy treatment does not comprise administration of a CD73 antagonist.

82. The method of any of claims 68 to 81, wherein the immunotherapy is an antagonist of a checkpoint inhibitor.

83. The method of any of claims 68 to 81, wherein the immunotherapy is an agonist of a checkpoint stimulator.

84. The method of claim 82, wherein the checkpoint inhibitor is an antagonist of the PD-1/PD-L1 axis (e.g., a PD-1 antagonist, a PD-L1 antagonist and a PD-L2 antagonist).

85. The method of claim 82, wherein the checkpoint inhibitor is an antagonist of CTLA-4, LAG-3, TIM3, TIGIT, VISTA, or B7/H3 (or another one described herein).

86. The method of claim 83, wherein the checkpoint stimulator is CD137, GITR, OX40, CD40, CD27, CD70 or ICOS (or another one described herein).

87. The method of any of claims 68-86, wherein the CD73 antagonist is a CD73 antibody.

88. The method of claim 87, wherein the CD73 antibody is any CD73 antibody described herein and/or described in any one of claims 1-67.

89. The method of claim 88, wherein the CD73 antibody is CD73.A.

90. The method of any of claims 68-89, wherein the CD73 antagonist is administered as described in any of claims 1-67.

91. A method for determining (or quantifying) the protein level of free sCD73 (i.e., not bound by an anti-CD73 therapeutic agent) in blood or serum of a human subject having received at least one dose of an anti-CD73 therapeutic agent, comprising contacting blood or serum of the subject with a solid surface comprising a first anti-CD73 agent, which competes with the CD73 therapeutic agent for binding to sCD73, under conditions and for an amount of time sufficient for the free sCD73 to bind to the anti-CD73 agent on the solid surface; washing the solid surface to remove unbound molecules, and detecting bound free sCD73 with a second anti-CD73 agent, which does not compete with the first anti-CD73 agent for binding to sCD73.

92. The method of claim 91, wherein the anti-CD73 therapeutic agent is CD73.A, and wherein the first anti-CD73 agent comprises the 6 CDRs, the heavy and light chain variable regions or the full length heavy and light chains of antibody 6E11 (comprising heavy and light chain sequences of SEQ ID NOs: 28 and 29, respectively).

93. The method of claim 91 or 92, wherein the second anti-CD73 agent comprises the 6 CDRs, the heavy and light chain variable regions or the full length heavy and light chains of antibody 4C3 (comprising heavy and light chain sequences of SEQ ID NOs: 38 and 39, respectively).

94. The method of any of claims 91 to 93, wherein the second anti-CD73 agent is labeled.

95. The method of claim 94, wherein the second anti-CD73 agent is ruthenylated.

96. A method for determining (or quantifying) the protein level of total sCD73 protein (i.e., bound or unbound by an anti-CD73 therapeutic agent) in blood or serum of a subject, comprising
(i) contacting blood or serum of the subject with a solid surface comprising an anti-CD73 agent, which does not compete for binding to sCD73 with the anti-CD73 therapeutic agent, under conditions and for an amount of time sufficient for the sCD73 to bind to the anti-CD73 agent on the solid surface;
(ii) eluting the sCD73 (bound or unbound by the anti-CD73 therapeutic agent);
(iii) trypsin digesting the eluted sCD73; and
(iv) subjecting the trypsin digested sCD73 to HPLC-MS/MS and determining the quantity of peptide VIYPAVEGR.

97. The method of claim 96, wherein the anti-CD73 therapeutic agent is CD73.A, and wherein the anti-CD73 agent comprises the 6 CDRs, the heavy and light chain variable regions or the full length heavy and light chains of antibody 4C3 (comprising heavy and light chain sequences of SEQ ID NOs: 38 and 39, respectively).


SEQUENCE LISTING (49 sequences)

SEQ ID NO: 1 - protein, 574 aa, Homo sapiens
  misc_feature: Human CD73 isoform 1
MCPRAARAPATLLLALGAVLWPAAGAWELTILHTNDVHSRLEQTSEDSSKCVNASRCMGGVARLFTKVQQIRRAEPNVLLLDAGDQYQGTIWFTVYKGAEVAHFMNALRYDAMALGNHEFDNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGHSGFEMDKLIAQKVRGVDVVVGGHSNTFLYTGNPPSKEVPAGKYPFIVTSDDGRKVPVVQAYAFGKYLGYLKIEFDERGNVISSHGNPILLNSSIPEDPSIKADINKWRIKLDNYSTQELGKTIVYLDGSSQSCRFRECNMGNLICDAMINNNLRHTDEMFWNHVSMCILNGGGIRSPIDERNNGTITWENLAAVLPFGGTFDLVQLKGSTLKKAFEHSVHRYGQSTGEFLQVGGIHVVYDLSRKPGDRVVKLDVLCTKCRVPSYDPLKMDEVYKVILPNFLANGGDGFQMIKDELLRHDSGDQDINVVSTYISKMKVIYPAVEGRIKFSTGSHCHGSFSLIFLSLWAVIFVLYQ

SEQ ID NO: 2 - protein, 524 aa, Homo sapiens
  misc_feature: Human CD73 isoform 2
MCPRAARAPATLLLALGAVLWPAAGAWELTILHTNDVHSRLEQTSEDSSKCVNASRCMGGVARLFTKVQQIRRAEPNVLLLDAGDQYQGTIWFTVYKGAEVAHFMNALRYDAMALGNHEFDNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGHSGFEMDKLIAQKVRGVDVVVGGHSNTFLYTGNPPSKEVPAGKYPFIVTSDDGRKVPVVQAYAFGKYLGYLKIEFDERGNVISSHGNPILLNSSIPEDPSIKADINKWRIKLDNYSTQELGKTIVYLDGSSQSCRFRECNMGNLICDAMINNNLRHTDEMFWNHVSMCILNGGGIRSPIDERNNGIHVVYDLSRKPGDRVVKLDVLCTKCRVPSYDPLKMDEVYKVILPNFLANGGDGFQMIKDELLRHDSGDQDINVVSTYISKMKVIYPAVEGRIKFSTGSHCHGSFSLIFLSLWAVIFVLYQ

SEQ ID NO: 3 - protein, 447 aa, Artificial Sequence
  Synthetic Heavy chain  CD73.A
QVQLVESGGGVVQPGRSLRLSCAASGFTFSNYGMHWVRQAPGKGLEWVAVILYDGSNKYYPDSVKGRFTISRDNSKNTLYLQMNSLRAEDTAVYYCARGGSSWYPDSFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 446 aa, Artificial Sequence
  Synthetic Heavy chain  CD73.A, without C-terminal lysine
QVQLVESGGGVVQPGRSLRLSCAASGFTFSNYGMHWVRQAPGKGLEWVAVILYDGSNKYYPDSVKGRFTISRDNSKNTLYLQMNSLRAEDTAVYYCARGGSSWYPDSFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 5 - protein, 214 aa, Artificial Sequence
  Synthetic Light chain  CD73.A
DIQMTQSPSSLSASVGDRVTITCRASQGISSWLAWYQQKPEKAPKSLIYAASSLQSGVPSRFSGSGSGTDFTLTISSLQPEDFATYYCQQYNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 121 aa, Artificial Sequence
  Synthetic Heavy chain variable region  CD73.A
QVQLVESGGGVVQPGRSLRLSCAASGFTFSNYGMHWVRQAPGKGLEWVAVILYDGSNKYYPDSVKGRFTISRDNSKNTLYLQMNSLRAEDTAVYYCARGGSSWYPDSFDIWGQGTMVTVSS

SEQ ID NO: 7 - protein, 107 aa, Artificial Sequence
  Synthetic Light chain variable region  CD73.A
DIQMTQSPSSLSASVGDRVTITCRASQGISSWLAWYQQKPEKAPKSLIYAASSLQSGVPSRFSGSGSGTDFTLTISSLQPEDFATYYCQQYNSYPLTFGGGTKVEIK

SEQ ID NO: 8 - protein, 5 aa, Artificial Sequence
  Synthetic HCDR1  CD73.A
NYGMH

SEQ ID NO: 9 - protein, 17 aa, Artificial Sequence
  Synthetic HCDR2  CD73.A
VILYDGSNKYYPDSVKG

SEQ ID NO: 10 - protein, 12 aa, Artificial Sequence
  Synthetic HCDR3  CD73.A
GGSSWYPDSFDI

SEQ ID NO: 11 - protein, 11 aa, Artificial Sequence
  Synthetic LCDR1  CD73.A
RASQGISSWLA

SEQ ID NO: 12 - protein, 7 aa, Artificial Sequence
  Synthetic LCDR2  CD73.A
AASSLQS

SEQ ID NO: 13 - protein, 9 aa, Artificial Sequence
  Synthetic LCDR3  CD73.A
QQYNSYPLT

SEQ ID NO: 14 - protein, 326 aa, Artificial Sequence
  Synthetic IgG2/IgG1 hybrid Fc
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 440 aa, Artificial Sequence
  Synthetic Heavy chain  nivolumab
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 401 aa, Artificial Sequence
  Synthetic Heavy chain  nivolumab without C-terminal lysine
QAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 17 - protein, 214 aa, Artificial Sequence
  Synthetic Light chain  nivolumab
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 113 aa, Artificial Sequence [sequence omitted]

SEQ ID NO: 19 - protein, 107 aa, Artificial Sequence [sequence omitted]

SEQ ID NO: 20 - protein, 5 aa, Artificial Sequence
  Synthetic HCDR1  nivolumab
NSGMH

SEQ ID NO: 21 - protein, 17 aa, Artificial Sequence
  Synthetic HCDR2  nivolumab
VIWYDGSKRYYADSVKG

SEQ ID NO: 22 - protein, 4 aa, Artificial Sequence
  Synthetic HCDR3  nivolumab
NDDY

SEQ ID NO: 23 - protein, 11 aa, Artificial Sequence
  Synthetic LCDR1  nivolumab
RASQSVSSYLA

SEQ ID NO: 24 - protein, 7 aa, Artificial Sequence
  Synthetic LCDR2  nivolumab
DASNRAT

SEQ ID NO: 25 - protein, 9 aa, Artificial Sequence
  Synthetic LCDR3  nivolumab
QQSSNWPRT

SEQ ID NO: 26 - protein, 19 aa, Artificial Sequence
  Synthetic CD73 epitope
FTKVQQIRRAEPNVLLLDA

SEQ ID NO: 27 - protein, 16 aa, Artificial Sequence
  Synthetic CD73 epitope
LYLPYKVLPVGDEVVG

SEQ ID NO: 28 - protein, 451 aa, Artificial Sequence
  Synthetic Heavy chain  6E11
EVQLVESGGALVQPGRSLRLSCAASGFTFDDYAMHWVRQAPGKGLEWVSGITWNSGGIGYADSVKGRFTISRDNAKNSLYLQMNSLRAEDTALYYCAKDRYYSSWLLFDNWGQGILVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 214 aa, Artificial Sequence
  Synthetic Light chain  6E11
EIVLTQSPGTLSLSPGERATLSCRASQSVSSSYLAWYQQKPGQAPRLLIYGASSRATGIPDRFSGSGSGTDFTLTISRLEPEDFAVYYCQHYGSSFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 121 aa, Artificial Sequence
  Synthetic Heavy chain variable region  6E11
EVQLVESGGALVQPGRSLRLSCAASGFTFDDYAMHWVRQAPGKGLEWVSGITWNSGGIGYADSVKGRFTISRDNAKNSLYLQMNSLRAEDTALYYCAKDRYYSSWLLFDNWGQGILVTVSS

SEQ ID NO: 31 - protein, 107 aa, Artificial Sequence
  Synthetic Light chain variable region  6E11
EIVLTQSPGTLSLSPGERATLSCRASQSVSSSYLAWYQQKPGQAPRLLIYGASSRATGIPDRFSGSGSGTDFTLTISRLEPEDFAVYYCQHYGSSFTFGPGTKVDIK

SEQ ID NO: 32 - protein, 5 aa, Artificial Sequence
  Synthetic HCDR1  6E11
DYAMH

SEQ ID NO: 33 - protein, 17 aa, Artificial Sequence
  Synthetic HCDR2  6E11
GITWNSGGIGYADSVKG

SEQ ID NO: 34 - protein, 12 aa, Artificial Sequence
  Synthetic HCDR3  6E11
DRYYSSWLLFDN

SEQ ID NO: 35 - protein, 12 aa, Artificial Sequence
  Synthetic LCDR1  6E11
RASQSVSSSYLA

SEQ ID NO: 36 - protein, 7 aa, Artificial Sequence
  Synthetic LCDR2  6E11
GASSRAT

SEQ ID NO: 37 - protein, 8 aa, Artificial Sequence
  Synthetic LCDR3  6E11
QHYGSSFT

SEQ ID NO: 38 - protein, 450 aa, Artificial Sequence
  Synthetic Heavy chain  4C3
EVQLVESGGGLVQPGRSLRLSCAASGFTFDDYAMHWVRQAPGKGLEWVSGISWKSGSIGYADSVKGRFTISRDNAKNSLYLQMNSLRAEDTALYYCVKGYYVILTGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 214 aa, Artificial Sequence
  Synthetic Light chain  4C3
EIVLTQSPGTLSLSPGERATLSCRASQSVSSYLAWYQQKPGQAPRLLIYGASSRATGIPDRFSGSGSGTDFTLTISRLEPEDFAVYYCQQYGSSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 120 aa, Artificial Sequence
  Synthetic Heavy chain variable region  4C3
EVQLVESGGGLVQPGRSLRLSCAASGFTFDDYAMHWVRQAPGKGLEWVSGISWKSGSIGYADSVKGRFTISRDNAKNSLYLQMNSLRAEDTALYYCVKGYYVILTGLDYWGQGTLVTVSS

SEQ ID NO: 41 - protein, 107 aa, Artificial Sequence
  Synthetic Light chain variable region  4C3
EIVLTQSPGTLSLSPGERATLSCRASQSVSSYLAWYQQKPGQAPRLLIYGASSRATGIPDRFSGSGSGTDFTLTISRLEPEDFAVYYCQQYGSSPLTFGGGTKVEIK

SEQ ID NO: 42 - protein, 5 aa, Artificial Sequence
  Synthetic HCDR1  4C3
DYAMH

SEQ ID NO: 43 - protein, 17 aa, Artificial Sequence
  Synthetic HCDR2  4C3
GISWKSGSIGYADSVKG

SEQ ID NO: 44 - protein, 11 aa, Artificial Sequence
  Synthetic HCDR3  4C3
GYYVILTGLDY

SEQ ID NO: 45 - protein, 12 aa, Artificial Sequence
  Synthetic LCDR1  4C3
RASQSVSSYLAW

SEQ ID NO: 46 - protein, 7 aa, Artificial Sequence
  Synthetic LCDR2  4C3
ASSRATG

SEQ ID NO: 47 - protein, 8 aa, Artificial Sequence
  Synthetic LCDR3  4C3
QYGSSPLT

SEQ ID NO: 48 - protein, 9 aa, Artificial Sequence
  Synthetic stable isotope-labeled surrogate peptide
  MOD_RES (1)..(1): SIL
VIYPAVEGR

SEQ ID NO: 49 - protein, 9 aa, Artificial Sequence
  Synthetic peptide
VIYPAVEGR